(19) United States
(12) Patent Application Publication
(10) Pub. No.: US 2015/0282934 A1
(43) Pub. Date: Oct. 8, 2015

(54) MULTILAYER POLYMERIC COMPOSITIONS AND METHODS RELATING THERETO

(71) Applicant: POLY-MED, INC., Anderson, SC (US)

(72) Inventors: Kenneth David Gray, JR., Clemson, SC (US); Joel Thomas Corbett, Anderson, SC (US); Georgios Theofanis Hilas, Anderson, SC (US); Seth Dylan McCullen, Greenville, SC (US)

(21) Appl. No.: 14/435,156

(22) PCT Filed: Oct. 11, 2013
(86) PCT No.: PCT/US13/64724

§ 371 Date: Apr. 10, 2015

Related U.S. Application Data
(60) Provisional application No. 61/712,632, filed on Oct. 11, 2012.

(51) Int. Cl. A61F 2/36; A61L 27/54; A61L 27/26

(52) U.S. Cl. CPC A61F 2/3609; A61L 27/26; A61L 27/54; A61F 2002/3611; A61F 2210/0076; A61L 2430/24; A61L 2430/06

(57) ABSTRACT

Multilayer polymeric materials in the form of a cap contain polymeric layers having distinctive anisotropic properties which are beneficially utilized in a subject, e.g., the multilayer polymeric materials are biocompatible and may be used, for example, to replace or supplement cartilage in an articulated joint. The present invention relates generally to multilayer polymeric materials, in particular to biomaterials suitable for implantation into a mammal or other animal, and methods of manufacturing the biomaterial and various uses thereof.

### CROSS-REFERENCE TO RELATED APPLICATION

This application claims the benefit under 35 U.S.C. §119(e) of U.S. Provisional Patent Application No. 61/712,632 filed Oct. 11, 2012, which application is incorporated herein by reference in its entirety.

### FIELD OF THE INVENTION

The present invention relates generally to multilayer polymeric materials, in particular to biomaterials suitable for implantation into a mammal or other animal, and methods of manufacturing the biomaterial and various uses thereof.

### BACKGROUND

Biocompatible polymeric materials are in increasing demand, particularly biomaterials that can be used to supplement or replace natural materials within a person that degrade upon aging, or need to be replaced upon injury. Within a person, the natural materials tend to be maintained by the body and thus are dynamic materials. A challenge with finding a synthetic bioequivalent is that the synthetic material is not nourished or physiologically supported by the host, and thus the bioequivalent is preferably inherently stable over a long period of time. Another challenge is that many implants are placed in stressful environments, i.e., environments that are under repeated mechanical stress, or are constantly exposed to biological fluids that can degrade the polymers. The polymeric materials must therefore be particularly durable. In some environments, it is necessary that the implant be able to absorb moisture from the surrounding biological fluids, which normally will tend to soften the polymer and make it less resistant to mechanical stress.

There is a need for biocompatible polymeric materials that can absorb moisture but still be durable upon exposure to repeated mechanical stress. The present invention is directed towards this need.

### SUMMARY

The following sets forth various exemplary aspects of the present invention and optional embodiments thereof.

In one aspect there is provided a biomimetic polymeric composition, specifically a multilayer polymeric composition, intended to mimic the mechanical properties of natural articular cartilage and to serve as a suitable substitute for articular cartilage.

In another aspect there is provided a multilayer composition comprising a first layer in contact with a second layer, the first layer comprising a first polymer and the second layer comprising a second polymer. The multilayer composition is advantageously used in an environment where the first layer is exposed to a different environmental condition than is the second layer. For this reason, the polymers of the first and second layers, i.e., the first and second polymers, are non-identical and are selected to provide different mechanical properties.

In another aspect there is provided a multilayer polymeric composition comprising a first layer in contact with a second layer, the first layer comprising a blend of polymers and the second layer comprising a different blend of polymers, i.e., a blend of polymers that is different from the blend of polymers present in the first layer. The multilayer composition is advantageously used in an environment where the first layer is exposed to a different environmental condition than is the second layer. For this reason, the polymers of the first and second layers, i.e., the first and second polymers, are non-identical and are selected to provide different mechanical properties.

In another aspect there is provided a multilayer composition in the form of a cap, the cap comprising a center and a rim, where the rim surrounds the center. At least one of the center and the rim comprises a first layer in contact with a second layer, the first layer comprising a first polymer or blend of polymers and the second layer comprising a second polymer or a second different blend of polymers. In one convention which may be used to describe any of the aspects and embodiments identified herein, the first layer is directly adjacent to the support and the second layer is adjacent to the first layer such that the first layer lies between the support and the second layer.

Optionally, the innermost layer (i.e., the layer directly adjacent to the head of the femoral bone or equivalent) of the multilayer composition can partially bioabsorbable, such as, for example, wherein the inner layer is a blend of (1) a polyether ester urethane urea (PEEUU) and (2) a biostable polyether urethane urea, where the polyether ester urethane urea degrades slowly over time to create a porous microstructure allowing for cellular ingrowth to provide improved fixation of the femoral cap to the femoral head; in further optional embodiments, growth factors, peptides, and/or pharmaceutical agents, for example, that promote osteogenesis and/or cartilage growth are incorporated within the innermost layer to facilitate the ingrowth of chrondrocytes and/or osteocytes. Suitable pore sizes range from 100-500 micrometers in diameter to allow for cellular ingrowth.

In another embodiment, the innermost layer can be formed by co-electrospinning a PEUU and a porogen, such as polyethylene glycol, or porogen fibers fabricated from PEEUU or absorbable, biocompatible, aliphatic polyester for the tailored ingrowth of bone or cartilage. The porogen diffuses out of the layer or degrades to create a porous microstructure allowing for cellular ingrowth to provide improved fixation of the femoral cap to the femoral head; in optional embodiments, growth factors that promote osteogenesis and cartilage growth are incorporated within the innermost layer to facilitate the ingrowth of chrondrocytes and/or osteocytes. Suitable pore sizes range from 100-500 micrometers in diameter to allow for cellular ingrowth. Suitable fiber diameters preferably range from 0.1 to 10 micrometers and are preferably greater than 1.0 micrometers.

In another aspect there is provided a multilayer composition comprising a cap in combination with a support. The support may be a bone, including a portion of a bone, for example a femoral head. The support may be made from bone or a non-bone material that is in the shape of a bone, for example it may be made from Teflon® polymer. An exemplary support is made from Teflon® polymer and is in the shape of the head of a femur, i.e., a femoral head. The cap should fit snugly around a portion of the support. The cap comprises a multilayer composition comprising a first layer in contact with a second layer, the first layer comprising a first polymer or blend of polymers and the second layer comprising a second polymer or a second different blend of polymers. In one convention used herein, the first layer is directly adjacent to the support and the second layer is adjacent to the first layer.

Optionally, the multilayer composition may be anisotropic in terms of compressive modulus and/or water absorption and/or hardness. The compressive modulus of the layers gradually decrease from the innermost deep layer to the outermost surface layer, while optionally and additionally the water absorption gradually increases from the deep layer to the surface layer, and optionally and additionally the Shore hardness gradually decreases from the innermost deep layer to the outermost surface layer.

In one embodiment, the compressive modulus decreases from the innermost deep layer to the outermost surface layer, and the compressive modulus ranges from 1-10 MPa in the innermost deep layer, 1-5 MPa in the middle layer, and 0.1-1 MPa in the outermost surface layer.

Tensile modulus: innermost layer ranges from 1-5 MPa, middle layer ranges from 5-25 MPa, and outermost surface layer ranges from 10-50 MPa. That is, tensile modulus increases from innermost layer to outermost layer.

In another aspect there is provided a method for forming a multilayer polymeric composition. This method includes: (a) providing a first solution comprising a first polymer or blend of polymers; (b) providing a second solution comprising a second polymer or a second different blend of polymers, the first and second polymers or blends being non-identical; (c) providing a support in the shape of a bone or a portion of a bone; (d) depositing first solution onto the support or portion thereof to provide a first coated support; (e) depositing additional first solution onto the first coated support a plurality of times to provide a first layer on the support or portion thereof; (f) depositing second solution onto the first layer to provide a second coated support; and (g) depositing additional second solution onto the second coated support a plurality of times to provide a second layer, the multilayer composition comprising the first layer and the second layer.

In another aspect there is provided a method for forming a multilayer polymeric composition. In this aspect, the method includes: (a) providing a first solution comprising a first polymer or blend of polymers; (b) providing a second solution comprising a second polymer or a second different blend of polymers, the first and second polymers or blends being non-identical; (c) providing a support in the shape of a bone or a portion of a bone; (d) depositing the first solution onto a portion of the support to provide a first coated support; (e) depositing the first solution onto the first coated support a plurality of times to provide a first layer on the portion of the support; (f) removing the first layer from the support; (g) inverting the first layer to provide an inverted first layer, and placing the inverted first layer onto the support to provide an inverted first support coating; (h) depositing the second solution onto the inverted first support coating to provide a second coated support; and (i) depositing the second solution onto the second coated support a plurality of times to provide the a second layer, the multilayer composition comprising the first layer and the second layer.

In another aspect there is provided a method for forming a multilayer polymeric composition. In this aspect, the method includes: (a) providing a first solution comprising a first polymer or blend of polymers; (b) providing a second solution comprising a pre-polymer of a second polymer or blend of polymers, the first and second polymers or blends being non-identical; (c) providing a third solution comprising a reactant reactive with the pre-polymer, the reactant and the pre-polymer being reactive to form the second polymer; (d) providing a fourth solution comprising a third polymer or blend of polymers; (e) providing a support in the shape of a bone or a portion thereof; (f) depositing the first solution onto the support or portion thereof to provide a first coated support; (g) depositing the first solution onto the first coated support a plurality of times to provide a first layer; (h) depositing the second solution on the first layer to provide a second coated support; (i) depositing the third solution on the second coated support to form an intermediate layer and provide a third coated support; (j) depositing the fourth solution onto the third coated support to provide a fourth coated support; and (k) depositing the fourth solution on the fourth coated support a plurality of times to provide a second layer, the multilayer composition comprising the first layer, the intermediate layer and the second layer.

In another aspect there is provided a method that includes: (a) receiving the dimensions of a bone, for example a bone that is present in a subject; and (b) preparing a multilayer composition that fits snugly on the bone. The multilayer comprises a first layer in contact with a second layer, the first layer comprising a first polymer or blend of polymers and the second layer comprising a second polymer or a second different blend of polymers. The multilayer composition is advantageously used in an environment where the first layer is exposed to a different environmental condition than is the second layer. For this reason, the polymers of the first and second layers, i.e., the first and second polymers or blends, are non-identical and are selected to provide different properties that optionally mimic the mechanical properties of the different zones in natural articular cartilage

In another aspect there is provided a method that includes: (a) receiving the dimensions of a bone, for example a bone that is present in a subject; and (b) preparing a multilayer composition that fits snugly on the bone. The multilayer composition may be in the form of a cap, the cap comprising a center and a rim, where the rim surrounds the center. At least one of the center and the rim comprises a first layer in contact with a second layer, the first layer comprising a first polymer or blend of polymers and the second layer comprising a second polymer or a second different blend of polymers.

In another aspect there is provided a method that includes: (a) receiving the dimensions of a bone, for example a bone that is present in a subject; and (b) preparing a multilayer composition that fits snugly on the bone. The multilayer polymeric composition may be prepared by a method that includes: (a) providing a first solution comprising a first polymer or blend of polymers; (b) providing a second solution comprising a second polymer or a second different blend of polymers, the first and second polymers or blends being non-identical; (c) providing a support in the shape of a bone or a portion of a bone; (d) depositing first solution onto the support or portion thereof to provide a first coated support; (e) depositing additional first solution onto the first coated support a plurality of times to provide a first layer on the support or portion thereof; (f) depositing second solution onto the first layer to provide a second coated support; and (g) depositing additional second solution onto the second coated support a plurality of times to provide a second layer, the multilayer composition comprising the first layer and the second layer.

In another aspect there is provided a method that includes: (a) receiving the dimensions of a bone, for example a bone that is present in a subject; and (b) preparing a multilayer composition that fits snugly on the bone. The multilayer polymeric composition may be prepared by a method that includes: (a) providing a first solution comprising a first polymer or blend of polymers; (b) providing a second solution comprising a second polymer or a second different blend of polymers, the first and second polymers or blends being non-identical; (c) providing a support in the shape of a bone or a portion of a bone; (d) depositing the first solution onto a portion of the support to provide a first coated support; (e) depositing the first solution onto the first coated support a plurality of times to provide a first layer on the portion of the support; (f) removing the first layer from the support; (g) inverting the first layer to provide an inverted first layer, and placing the inverted first layer onto the support to provide an inverted first support coating; (h) depositing the second solution onto the inverted first support coating to provide a second coated support; and (i) depositing the second solution onto the second coated support a plurality of times to provide the a second layer, the multilayer composition comprising the first layer and the second layer.

In another aspect there is provided a method that includes: (a) receiving the dimensions of a bone, for example a bone that is present in a subject; and (b) preparing a multilayer composition that fits snugly on the bone. The multilayer polymeric composition may be prepared by a method that includes: (a) providing a first solution comprising a first polymer or blend of polymers; (b) providing a second solution comprising a pre-polymer of a second polymer or blend of polymers, the first and second polymers being non-identical; (c) providing a third solution comprising a reactant reactive with the pre-polymer, the reactant and the pre-polymer being reactive to form the second polymer; (d) providing a fourth solution comprising a third polymer or blend of polymers; (e) providing a support in the shape of a bone or a portion thereof; (f) depositing the first solution onto the support or portion thereof to provide a first coated support; (g) depositing the first solution onto the first coated support a plurality of times to provide a first layer; (h) depositing the second solution on the first layer to provide a second coated support; (i) depositing the third solution on the second coated support to form an intermediate layer and provide a third coated support; (j) depositing the fourth solution onto the third coated support to provide a fourth coated support; and (k) depositing the fourth solution on the fourth coated support a plurality of times to provide a second layer, the multilayer composition comprising the first layer, the intermediate layer and the second layer.

In another aspect there is provided a kit. The kit includes a multilayer composition, which may optionally be in the form of a cap, which may optionally be prepared by methods disclosed herein. In addition to the multilayer composition, the kit may include one or more of an anesthetic, a cutting instrument that may be used by the surgeon to cut the patient receiving the multilayer composition in order to provide access to site within the patient where the multilayer composition is desirably placed, a support for the multilayer composition that holds the composition in a configuration which is helpful to the surgeon during the time when the multilayer composition is transferred from the kit to the desired location within the patient, and one or more other components that are usefully present in the kit.

Optionally, in each of the compositions and methods and kits disclosed herein, one or more the following criteria may be used to characterize the multilayer composition or a component thereof: the first polymer comprises a plurality of urethane groups; the second polymer comprises a plurality of urethane groups; the first and second polymers are non-identical. When the first and/or second polymer contains a plurality of urethane groups, one or both of the first and second polymers may additionally comprise repeating oxyalkylene groups (i.e., a polyether) and repeating linkages selected from urethane and urea. Such polymers are referred to herein as PEU, where exemplary PEUs include polyether urethane (PEUT), polyether urea (PEUA), polyether urea urethane (PEUU), polyether carbonate urethane (PECUT), polyether carbonate urea (PECUA), polyether carbonate urethane urea (PECUU), polyether ester urethane (PEEUT), polyether ester urea (PEEUA), and polyether ester urethane urea (PEEUU).

PEUs may be manufactured by various methods. One such method includes reacting an aliphatic or aromatic diisocyanate with a diol or diamine. Either of the diol or diamine may (or may not) incorporate additional functionality, e.g., carbonate or ester functionality. The product of this reaction may optionally be “chain extended” by reaction with additional diol and/or diamine. The diol or diamine used for the chain extension may (or may not) incorporate additional functionality, e.g., carbonate or ester functionality. The PEU will, however, contain some polyether functionality, and may optionally contain a plurality of carbonate or ester groups in addition to a plurality of urethane and/or urea groups.

Optionally, in each of the compositions and methods and kits disclosed herein, one or more the following criteria, which are phrased in terms of physical properties rather than chemical composition, may be used to characterize the multilayer composition or a component thereof. A layer (e.g., a first layer, a second layer, a third layer, etc.) or a polymer (e.g., a first polymer, a second polymer, a third polymer, etc.) may be characterized in terms of a maximum value, or a minimum value, or a range of values for a physical property within which the layer or polymer is characterized, or a relative value, e.g., the first polymer is more (insert physical property) than the second polymer. Exemplary physical properties referred to in the previous sentence include, without limitation, hydrophilicity or hydrophobicity or water absorption, elasticity, hardness, surface modulus, coefficient of friction, compressive modulus, toughness, tensile strength, and lubricity.

Optionally, in each of the compositions and methods and kits disclosed herein, one or more the following criteria, which describe the multilayer composition and/or the relationship between polymers and/or layers, may be used to characterize the multilayer composition or a component thereof: a multilayer composition comprising a first layer in contact with a second layer, the first layer comprising a first polymer and the second layer comprising a second polymer, the first polymer comprising a plurality of urethane or urea groups, the second polymer comprising a plurality of urethane or urea groups, the first and second polymers being non-identical, where optionally: the first polymer further comprises a plurality of urea groups; the first polymer further comprises a plurality of ether groups; the first polymer further comprises a plurality of carbonate groups; the first layer is a blend of polymers and the second layer is a different blend of polymers; the first layer is more hydrophilic than the second layer; the first layer is less hydrophilic than the second layer; the first layer is more elastic than the second layer; the first layer is less elastic than the second layer; the first layer has a greater hardness than the second layer; the first layer has a lower compressive modulus than the second layer; the first layer has a higher tensile modulus than the second layer; the first layer has a higher surface modulus than the second layer; the first layer has a greater toughness than the second layer; the first layer has a higher Tg than the second layer; the first layer has a lower Tg than the second layer; the first layer has a thickness of 0.1 to 1.5 mm; the first layer has a thickness of 0.1 to 1.0 mm; the first layer has a thickness of 0.1 to 0.5 mm; the first layer makes up approximately 10-20% of the composition's thickness; the second layer makes up approximately 80-90% of the composition's thickness; the composition has exactly two layers; the composition further comprises a third layer in contact with the second layer, the third layer comprising a third polymer, the third polymer comprising a plurality of urethane groups, the second and third polymers being non-identical, where in optional embodiments, the composition has exactly three layers, the first and third polymers are identical, the first and third polymers are non-identical, any or all of the layers can be a blend of polymers, the second layer is more hydrophilic than the first layer, the second layer is more hydrophilic than either the first layer or the third layer, the second layer is more hydrophilic than the first layer, the second polymer contains a greater weight percent of ether linkages compared to the first polymer and the second polymer is more hydrophilic than either the first polymer or the third polymer, the second polymer contains a greater weight percent of ether linkages compared to the first polymer and the second polymer is more hydrophilic than the first polymer, the second layer is more elastic than either the first layer or the third layer; the second layer is more elastic than the first layer; the first layer has a greater hardness than the second layer; the first layer has a lower compressive modulus than the second layer; the first layer has a higher tensile modulus than the second layer; the first layer has a higher surface modulus than the second layer; the first layer has a greater toughness than the second layer; the first layer contains a pharmaceutically active agent; the second layer contains a pharmaceutically active agent; the second layer has a greater hardness than the third layer; the second layer has a lower compressive modulus than the third layer; the second layer has a higher tensile modulus than the third layer; the second layer has a higher surface modulus than the third layer; the second layer has a greater toughness than the third layer; the first layer makes up approximately 10% to 20% of the composition's thickness; the second layer makes up approximately 30% to 70% of the composition's volume; the third layer makes up approximately 20-40% of the composition's volume; the composition has a thickness of 1.0 to 4.0 mm; the composition has a thickness of 1.0 to 3.0 mm; the composition has a thickness of 1.5 to 2.0 mm; the composition is marked; the composition is sterile; the composition has a longest straight line dimension of 1 to 5 cm or is in the shape of a cap having a half-circumference of 10-200 mm.

Some specific and exemplary embodiments of the present disclosure are as follows, some of which make reference to earlier numbered embodiments:

1. A multilayer composition in the form of a cap, the cap comprising a center and a rim around the center, the cap comprising an interior surface and an exterior surface, at least one of and preferably both of the center and the rim comprising a first layer in contact with a second layer, the interior surface comprising the first layer, the first layer comprising a first polymer, the second layer comprising a second polymer, the first polymer comprising a plurality of urethane or urea groups, the second polymer comprising a plurality of urethane or urea groups, the first and second polymers being non-identical.

2. A multilayer composition in the form of a cap, the cap comprising a center and a rim around the center, the cap comprising an interior surface and an exterior surface, at least one of, and preferably both of the center and the rim comprising a first layer in contact with a second layer, the interior surface comprising the first layer, the first layer comprising a blend of polymers, the second layer comprising a second different blend of polymers, the polymers of the blend forming the first layer comprising a plurality of urethane or urea groups, the polymers of the blend forming the second layer comprising a plurality of urethane or urea groups, the first and second polymer blends being non-identical.

3. The composition of embodiments 1 or 2 wherein the cap has a longest exterior dimension of 10 to 200 mm, the center has a thickness of 1.5 to 2.0 mm, and the rim has a thickness of 0.5 to 1.5 mm.

4. The composition of embodiments 1 or 2 wherein the first layer has a compressive modulus ranging from 0.1-1.0 MPa, and the second layer has a compressive modulus ranging from 1.0-10 MPa.

5. The composition of embodiments 1 or 2 wherein the first layer makes up 10-20% of the thickness of the construct, and the second layer makes up 80-90% of the thickness of the construct.

6. The composition of embodiments 1 or 2 wherein the first layer relative to the second layer is at least one of:
(a) is less hydrophilic;
(b) has a greater hardness;
(c) has a higher compressive modulus; and/or
(d) has a higher Tg.

7. The composition of embodiments 1 or 2 having exactly two layers.

8. The composition of embodiments for 2 wherein the second polymer contains a greater weight percent of ether linkages compared to the first polymer, and wherein the second polymer is more hydrophilic than the first polymer.

9. The composition of embodiments for 2 wherein the second layer comprises a blend of polymers, the combination of which has a greater weight percent of ether linkages compared to the blend of polymers in the first layer, and wherein the second layer is more hydrophilic than the first layer.

10. The composition of embodiments 1 or 2 wherein the inner surface contains a pharmaceutically active agent.

11. The composition of embodiment 1 further comprising a third layer in contact with the second layer, the third layer comprising a third polymer, the third polymer comprising a plurality of urethane groups, the second and third polymers being non-identical.

12. The composition of embodiment 2 further comprising a third layer in contact with the second layer, the third layer comprising a third blend of polymers, the third blend comprising a plurality of urethane groups, the second and third blends being non-identical.

13. The composition of embodiments 1 or 2 wherein both of the cap and the rim has exactly three layers.

14. A multilayer composition in the form of a cap, the cap comprising a center and a rim around the center, the cap comprising an interior surface, an intermediate layer, and an exterior surface, at least one of the center and the rim comprising a first layer in contact with a second layer, and a second layer in contact with a third layer, the interior surface comprising the first layer, the intermediate layer comprising the second layer, the outer layer comprising the third layer, the first layer comprising a blend of polymers, the second layer comprising a second different blend of polymers, the third layer comprising a third different blend of polymers, the polymers of the blends forming the first, second and third layers comprising a plurality of urethane or urea groups, the first, second, and third polymer blends being non-identical.

15. An assembly comprising a multilayer sheet-like composition in the form of a cap in combination with a support, the support selected from the group consisting of a bone and a material in the shape of a bone, wherein the cap fits snugly around a portion of the support, the cap comprising a center and a rim around the center, the cap comprising an interior surface and an exterior surface, at least one of the center and the rim comprising a first layer in contact with a second layer, the interior surface comprising the first layer, the first layer comprising a first polymer or blend of polymers, the second layer comprising a second polymer or a second blend of polymers, the first polymer or first blend of polymers comprising a plurality of urethane or urea groups, the second polymer or second blend of polymers comprising a plurality of urethane or urea groups, the first and second polymers or blends of polymers being non-identical.

16. The assembly of embodiment 15 wherein the support is in the shape of a head of a femur bone.

17. A multilayer composition in the form of a cap, comprising:
(a) a first layer, a second layer and a third layer, the second layer being located intermediate between the first and second layers, each layer having a compressive modulus and a hydrophilicity;
(b) the compressive modulus of the first layer being greater than the compressive modulus of the second layer, and the compressive modulus of the second layer being greater than the compressive modulus of the third layer; and
(c) the hydrophilicity of the first layer being less than the hydrophilicity of the second layer, and the hydrophilicity of the second layer being less than the hydrophilicity of the third layer; and
(d) the tensile modulus of the first layer being greater than the tensile modulus of the second layer, and the tensile modulus of the second layer being greater than the tensile modulus of the third layer; and

18. A method for forming a multilayer composition in the form of a cap, the method comprising:
(a) providing a first solution comprising a first polymer or blend of polymers;
(b) providing a second solution comprising a second polymer or a second blend of polymers, the first and second polymers or first and second blends of polymers being non-identical;
(c) providing a support in the shape of a bone or a portion of a bone;
(d) depositing the first solution onto the support or a portion thereof to provide a first coated support;
(e) depositing the first solution onto the first coated support a plurality of times to provide a first layer on the support or a portion thereof;
(f) depositing the second solution onto the first layer to provide a second coated support; and
(g) depositing the second solution onto the second coated support a plurality of times to provide a second layer, the multilayer composition comprising the first layer and the second layer.

19. The method of embodiment 18 wherein the support or a coated support is dipped into a first solution or a second solution to provide the depositing.

20. The method of embodiment 18 wherein the support or coated support is spun while it is being dipped into the first solution or the second solution.

21. A method for forming a multilayer composition in the form of a cap, the method comprising
(a) providing a first solution comprising a first polymer or blend of polymers;
(b) providing a second solution comprising a second polymer or blend of polymers, the first and second polymers or first and second blend of polymers being non-identical;
(c) providing a support in the shape of a bone or a portion of a bone;
(d) depositing the first solution onto a portion of the support to provide a first coated support;
(e) depositing the first solution onto the first coated support a plurality of times to provide a first layer on the portion of the support;
(f) removing the first layer from the support;
(g) inverting the first layer to provide an inverted first layer, and placing the inverted first layer onto the support to provide an inverted first support coating;
(h) depositing the second solution onto the inverted first support coating to provide a second coated support; and
(i) depositing the second solution onto the second coated support a plurality of times to provide a second layer, the multilayer composition comprising the first layer and the second layer.

22. A method for forming a multilayer composition in the form of a cap, the method comprising
(a) providing a first solution comprising a first polymer or blend of polymers;
(b) providing a second solution comprising a pre-polymer of a second polymer, the first and second polymers being non-identical;
(c) providing a third solution comprising a reactant reactive with the pre-polymer, the reactant and the pre-polymer being reactive to form the second polymer;
(d) providing a fourth solution comprising a third polymer or blend of polymers;
(e) providing a support in the shape of a bone or a portion thereof;
(f) depositing the first solution onto the support or a portion thereof to provide a first coated support;
(g) depositing the first solution onto the first coated support a plurality of times to provide a first layer;
(h) depositing the second solution on the first layer to provide a second coated support;
(i) depositing the third solution on the second coated support to form an intermediate layer and provide a third coated support;
(j) depositing the fourth solution onto the third coated support to provide a fourth coated support; and
(k) depositing the fourth solution on the fourth coated support a plurality of times to provide a second layer, the multilayer composition comprising the first layer, the intermediate layer and the second layer.

23. The method of embodiment 22 wherein the prepolymer comprises urethane groups and isocyanate end groups.

24. The method of embodiment 22 wherein the prepolymer comprises urethane groups, carbonate groups, and amine end groups.

25. A method comprising:
(a) receiving the dimensions of a bone in a subject; and
(b) preparing a multilayer sheet-like composition in the form of a cap according to claim 1 that fits snugly on the bone.

26. A kit comprising
(a) a multilayer composition in the form of a cap according to claim 1; and
(b) a solution that comprises an anesthetic.

The details of one or more aspects and embodiments are set forth in the description below. Other features, objects and advantages will be apparent from the description and the claims. In addition, the disclosures of all patents and patent applications referenced herein are incorporated by reference in their entirety.

### BRIEF DESCRIPTION OF THE DRAWINGS

Features of the present disclosure, its nature and various advantages will be apparent from the accompanying drawings and the following detailed description of various embodiments.

FIGS. 1A, 1B, 1C and 1D illustrates a typical human femur, providing two oppositing side views (FIGS. 1A and 1B), a front view (FIG. 1C) and a rear view (FIG. 1D).

FIGS. 2A and 2B illustrates a portion of a human femur, specifically identifying the head and neck of the femur (FIG. 2A), and the coupling of that femur portion with the acetabulum of the pelvic girdle (FIG. 2B).

FIG. 3A illustrates a cap of the present disclosure seated on the head and neck of the femur bone, while FIG. 3B illustrates a cap of the present invention positioned on a femur bone while that femur bone is coupled to the acetabulum. The cap is hollow so that it can sit on top of and surround all or part of the head of the bone.

FIG. 4 illustrates a cross-sectional view of a cap of the present disclosure having two layers. The cap is hollow, i.e., no material is present in the middle of the cap.

FIG. 5 illustrates a cross-sectional view of a cap of the present disclosure having three layers. The cap is hollow, i.e., no material is present in the middle of the cap.

### DETAILED DESCRIPTION OF THE INVENTION

### Terminology

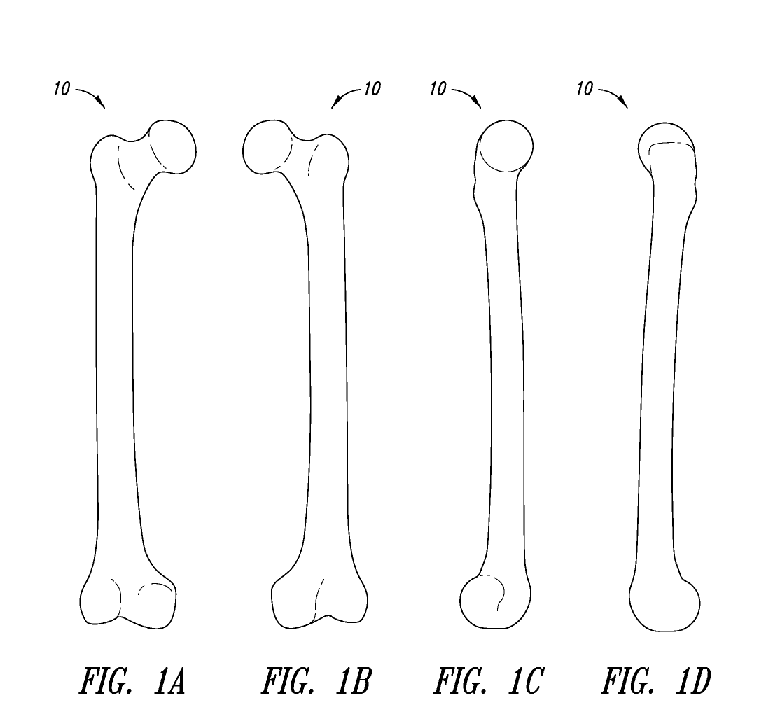

FIGS. 1A, 1B, 1C and 1D show the human femur bone 10, also called the thigh bone, from the back, front, and two sides of the bone.

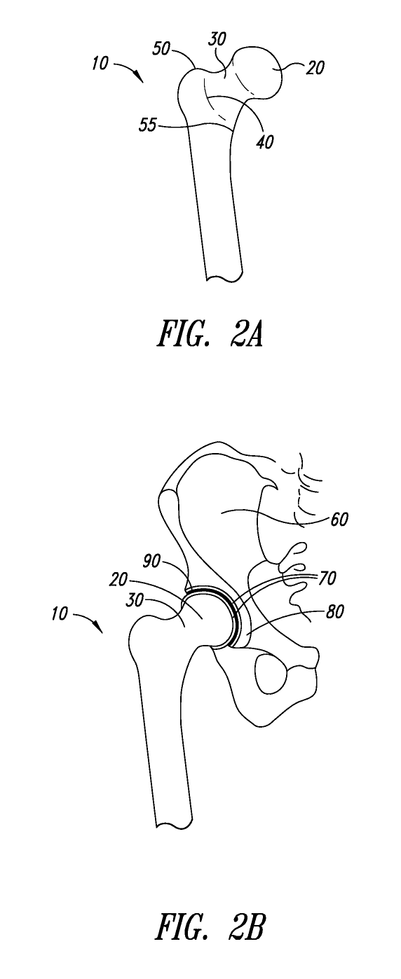

FIG. 2A shows the upper portion of the femur bone 10, and identifies parts thereof as follows: 20 is the head, 30 is the neck, 40 is the intertrochanteric crest and 50 is the greater trochanter and 55 is the lesser trochanter.

FIG. 2B shows how the femur bone 10 fits into the pelvic girdle 60 and identifies parts thereof: 20 is the head of the femur bone, 30 is the neck of the femur bone, 60 is the pelvic girdle, 70 is a layer of cartilage, where there is a cartilage layer 70 adjacent the head of the femur 20 and a cartilage layer 70 adjacent to the acetabulum 80 of the pelvic girdle 60. Between the two layers of cartilage 70 is a fluid layer 90, commonly known as synovial fluid.

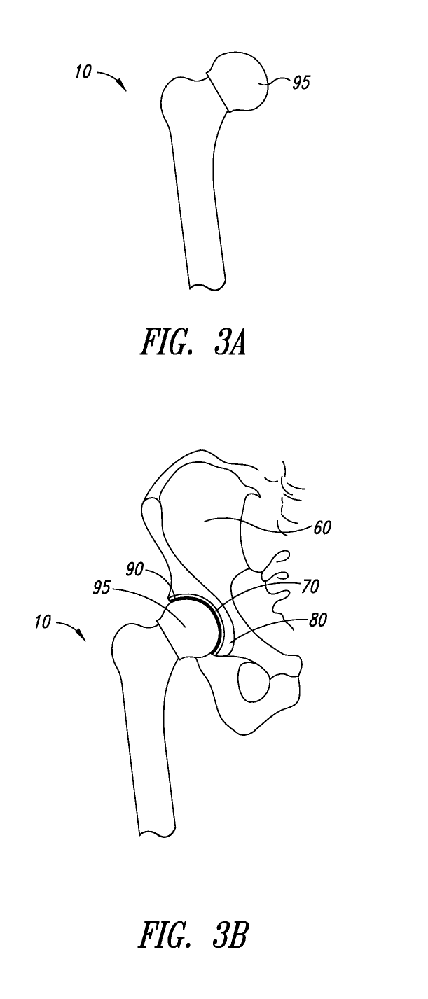

FIG. 3A shows a hollow cap 95 of the present disclosure which has been fitted around the head and part of the neck of a femur bone. The cap functions somewhat like a sock which slips over the foot, but in this case the cap slips over the head of the femur bone.

FIG. 3B shows how the capped femur bone of FIG. 3A may be inserted into the acetabulum of the pelvic girdle.

### Multilayer Compositions and Desired Shapes

In one aspect there is provided a multilayer composition comprising a first layer in contact with a second layer, the first layer comprising a first polymer and the second layer comprising a second polymer. The multilayer composition is advantageously used in an environment where the first layer is exposed to a different environmental condition than is the second layer. For this reason, the polymers of the first and second layers, i.e., the first and second polymers, are non-identical and are selected to provide different properties. For example, the present disclosure provides multilayer composition comprising a first layer in contact with a second layer, the first layer comprising a first polymer and the second layer comprising a second polymer, the first polymer comprising a plurality of urethane or urea groups, the second polymer comprising a plurality of urethane or urea groups, the first and second polymers being non-identical. As used herein, the term “comprising a plurality of urethane or urea groups” means that the referenced polymer contains at least a plurality of urethane groups or a plurality of urea groups or a plurality of both urea and urethane groups, where any one of these three options may be substituted herein for the term “comprising a plurality of urethane or urea groups” wherever it appears. Optionally, one or more of the descriptions set forth below may be used to further characterize the multilayer composition and/or a component thereof.

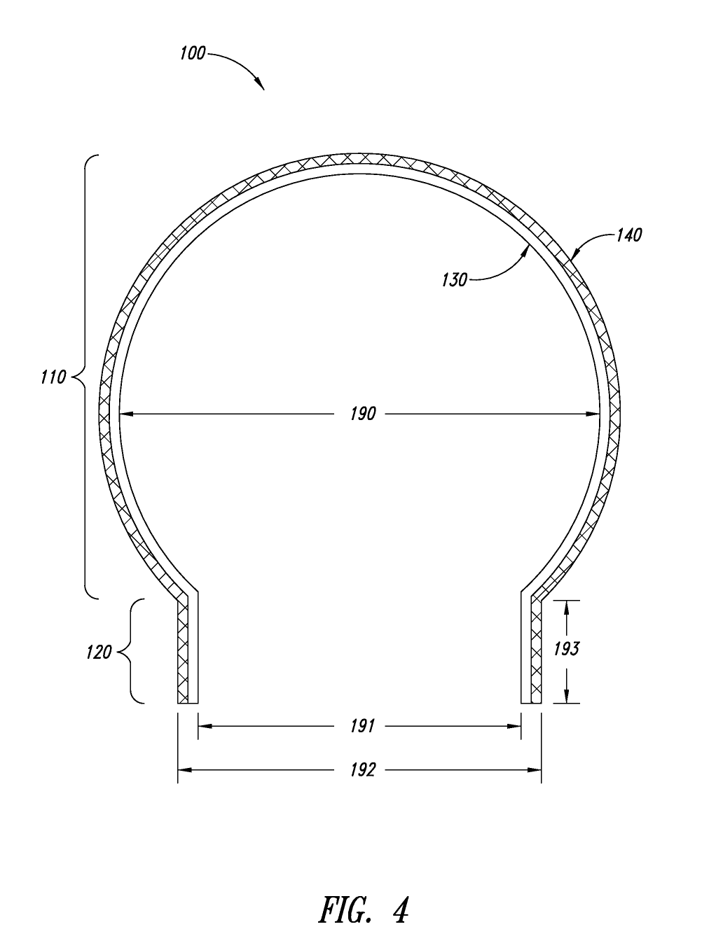

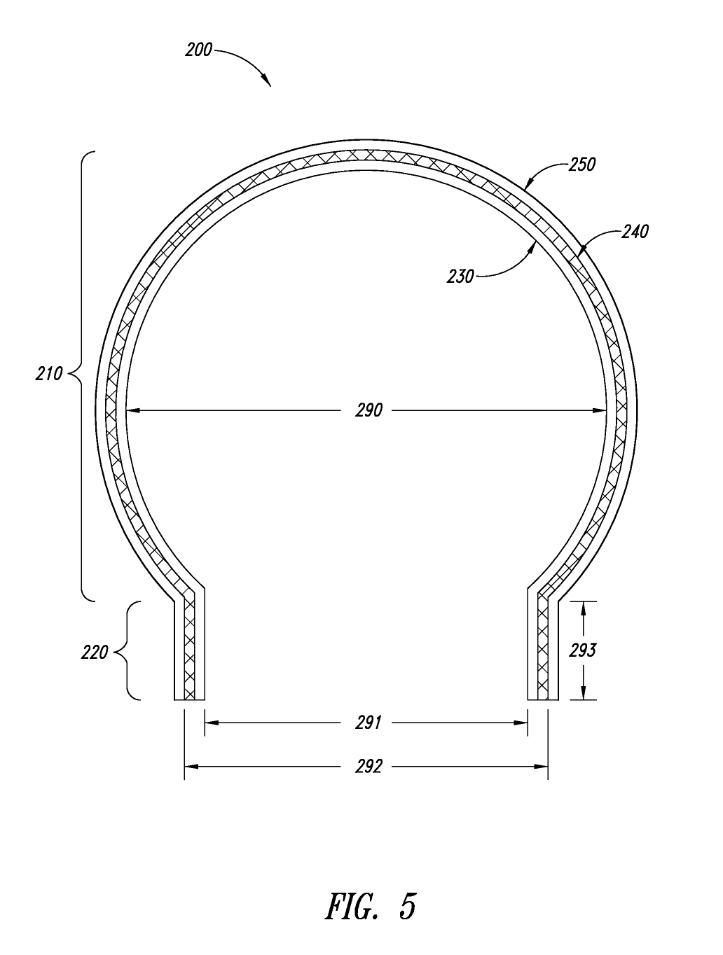

In another aspect there is provided a multilayer composition in the form of a cap, where exemplary caps are illustrated in FIG. 4 and FIG. 5. Looking first at FIG. 4, this figure shows a two layer cap 100 of the present invention. The cap 100 includes a center portion 110 and a rim (or neck) portion 120. The cap is formed from two polymer layers: an innermost layer 130 and an outer layer 140. The innermost layer forms the entirety of the inside of the cap while the outermost layer forms the entirety of the exterior of the cap. The center of the cap, which is hollow to allow the cap to surround a bone or equivalent, has an inner diameter 190 which is approximately equal to the diameter of the head of a femur bone. The cap 100 also has a rim, or neck of inner diameter 191, outer diameter 192 and height 193. The rim, while shown in FIGS. 4 and 5, is an optional feature of the cap of the present invention. Optionally, the rim may be omitted and the center region alone fitted onto the femur bone. Thus, in one embodiment, the present disclosure provides a multilayer composition in the form of a hollow cap, the cap comprising a center and optionally a rim that surrounds the center, at least one and preferably both, when present, of the center and the rim comprising a first layer in contact with a second layer, the first layer comprising a first polymer and the second layer comprising a second polymer, the first polymer comprising a plurality of urethane or urea groups, the second polymer comprising a plurality of urethane or urea groups, the first and second polymers being non-identical.

Another cap of the present invention is illustrated in FIG. 5. In FIG. 5, the cap is a trilayer cap. The cap 200 includes a hollow center portion 210 and is shown with a rim (or neck) 220 which is an optional feature of the cap. The cap is formed from three polymer layers: an innermost layer 230, an intermediate layer 240 and an outer layer 250. The center of the cap has an inner diameter 290 which is approximately equal to the diameter of the head of a femur bond. The cap 200 has an optional rim or neck of inner diameter 291, outer diameter 292 and height 293.

The cap will have the approximate appearance of a swimmer's cap: approximately half-spherical. The cap is designed to fit over and fit snugly around the end or head of a bone, where the end typically has a spherical or knobby shape as shown in FIG. 1. The cap may not be perfectly half-spherical, particularly if the end of the bone to be surrounded by the cap is not perfectly half-spherical. Also, the rim of the cap may or may be present, and when present, may not sit on the head of the bone precisely at the widest diameter of the head, but alternatively may not quite reach the widest diameter of the head, or may surpass the widest diameter of the head and extend beyond that widest diameter onto the neck of the bone. In either case, the cap is approximately half-spherical, having an open portion to allow the cap to be fitted onto a bone head.

The diameter of the mold or support from which the cap is formed, and hence the diameter of the cap at its widest possible point, is in the range of 25-75 mm when the mold is intended to mimic a femoral head, where a small femoral head has a diameter on the order of 30 mm while a larger femoral head has a diameter on the order of 60 mm. This distance is shown as the distance 190 in FIGS. 4 and 290 in FIG. 5. The cap may also be characterized by its half-circumference, which refers to the distance of a line from one point on the rim, through the center point of the cap, and back down to the opposite point on the rim. The half-circumference of a cap of the present disclosure is in the range of 30 to 130 mm when the cap is intended for a femoral head. When the cap is intended to fit onto a larger bone, i.e., larger than a femoral head, then the cap will have a size approximately the same as the larger bone, e.g., it may have a half-circumference of up to 150 mm or up to 170 mm or up to 200 mm. Likewise, when the cap is intended to fit onto a smaller bone, i.e., smaller than a femoral head, then the cap will have a size approximately the same as the smaller bone, e.g., it may have a half-circumference of as small as 25 mm or 20 mm or 15 mm or 10 mm. The cap may be of a suitable size for a human, either child or adult, or for an animal, e.g., a dog or a horse. In various embodiments, the cap has a half-circumference of 30-110 mm, or 40-100 mm, or 45-95 mm, or 50-90 mm.

One or more of the descriptions set forth herein may be used to further characterize the multilayer composition in the form of a cap and/or a component thereof.

In another aspect there is provided a multilayer composition comprising a cap in combination with a support. The support may be a bone, including a portion of a bone, or a non-bone material that is in the shape of a bone, again including a portion of a shape of a bone. The cap should fit snugly around a portion of the support. The cap comprises a multilayer composition comprising a first layer in contact with a second layer, the first layer comprising a first polymer and the second layer comprising a second polymer. For example, the present disclosure provide a composition comprising a cap in combination with a support, the support selected from the group consisting of a bone and a material in the shape of a bone, wherein the cap fits snugly around a portion of the support, and the cap comprises a multilayer composition comprising a first layer in contact with a second layer, the first layer comprising a first polymer and the second layer comprising a second polymer, the first polymer comprising a plurality of urethane or urea groups, the second polymer comprising a plurality of urethane or urea groups, the first and second polymers being non-identical. The support will have the shape of at least the end or head of a bone. In one embodiment, the support will have a spherical shape, or at least an approximately spherical shape to match the shape of the end of a bone. In one embodiment, the diameter of the support is in the range of 25-75 mm when the support is intended to mimic a femoral head, where a small femoral head has a diameter on the order of 30 mm while a larger femoral head has a diameter on the order of 60 mm. The support may be smaller or larger than this range, so long as it has a bone-sized dimension. Optionally, one or more of the descriptions set forth herein may be used to further characterize the multilayer composition in the form of a cap and/or a component thereof.

As mentioned previously, optionally, one or more of the following descriptions may be used to further characterize the multilayer composition and/or a component thereof: the first polymer comprises a plurality of urethane groups; the second polymer comprises a plurality of urethane groups; the first and second polymers are non-identical; the first polymer further comprises a plurality of urea groups; the first polymer further comprises a plurality of ether groups; the first polymer further comprises a plurality of carbonate groups; the first layer is more hydrophilic than the second layer; the first layer is less hydrophilic than the second layer; the first layer has a greater percent water absorption than the second layer; the first layer has a lesser percent water absorption than the second layer; the first layer is more elastic than the second layer; the first layer is less elastic than the second layer; the first layer has a greater hardness than the second layer; the first layer has a higher surface modulus than the second layer; the first layer has a greater toughness than the second layer; the first layer has a higher Tg than the second layer; the first layer has a lower Tg than the second layer; the first layer has a thickness of 0.1 to 1.5 mm; the first layer has a thickness of 0.1 to 1.0 mm; the first layer has a thickness of 0.1 to 0.5 mm; the multilayer composition has exactly two layers; the multilayer composition further contains a third layer in contact with the second layer, the third layer comprising a third polymer, the third polymer comprising a plurality of urethane groups, the second and third polymers being non-identical; the multilayer composition has exactly three layers where optionally the first and third polymers are identical or the first and third polymers are non-identical; the third polymer contains a higher weight percent of oxyethylene units than the second polymer, and the second polymer contains a higher weight percent of oxyethylene units than the first polymer; the first polymer has a greater weight percent of urethane and urea linkages than the second polymer, where the second polymer has a greater weight percent of urethane and urea linkages than the third polymer; the second layer is more hydrophilic than either the first layer or the third layer; the third layer is more hydrophilic than either the first layer or the second layer; the second polymer contains a greater weight percent of ether linkages compared to the first polymer, and wherein the second polymer is more hydrophilic than either the first polymer or the third polymer; the second layer is more elastic than either the first layer or the third layer; the third layer is more elastic than either the first or second layer while the second layer is more elastic than the first layer; the first layer has a greater hardness than the second layer; the first layer has a higher surface modulus than the second layer; the first layer has a greater toughness than the second layer; the third polymer contains a higher weight percent of polyethylene glycol than the second polymer, and the second polymer contains a higher weight percent of polyethylene than the first polymer; the first layer contains a pharmaceutically active agent; the second layer contains a pharmaceutically active agent; the composition has a thickness of 1.0 to 3.0 mm; the composition has a thickness of 1.5 to 2.0 mm; the composition is tinted; the composition is sterile; the composition has a longest straight line dimension of 1 to 5 cm, the composition is in the form of a cap having a half-circumference of 10-200 mm.

Accordingly, in one embodiment the cap of the present disclosure may have more than the two layers shown in FIG. 4. For example, the cap of the present disclosure may have three layers, where this embodiment is illustrated in FIG. 5. The cap 200 of FIG. 5 includes a center portion 210 and an optional rim portion 220. The cap comprises an innermost layer 230, an adjacent intermediate layer 240 and an outermost layer 250. In common with the bilayer cap illustrated in FIG. 4, the trilayer cap of FIG. 5 includes an optional rim of length 293, having an inner diameter 291 and an outer diameter 292. In a preferred embodiment, the innermost layer of the center portion is the same material as the innermost layer of the rim (when present). Likewise, the rim and center portion may have the same intermediate layer and outermost layer, in terms of the polymer(s) from which those layers are formed.

The first and/or second and/or third etc. polymer may also, or alternatively, optionally be characterized by one or more of the following characterizations:

a. at least one of the polymers is the reaction product of a pre-polymer and a diamine, where the pre-polymer is the reaction product of a diisocyanate and a polyetherdiol, where optionally the polyetherdiol comprises at least one type of oxyalkylene sequence selected from the group consisting of oxyethylene, oxypropylene, oxytrimethylene and oxytetramethylene sequences and/or the polyetherdiol is a blend of polyetherdiols or the polyether diol is not a blend of polyetherdiols, and/or the polyetherdiol is a random copolymer of two or more oxyalkylene sequences, and/or the polyetherdiol is a block copolymer of two or more oxyalkylene sequences, and/or the polyether diol is an alternating copolymer of two or more oxyalkylene sequences, and/or the polyether diol is an alternating copolymer of two different oxyalkylene repeat units, e.g., oxyethylene and oxypropylene; the diamine is an aliphatic diamine; the diamine is a polyether diamine; the diamine is a blend of diamines; the diamine is a blend of aliphatic diamine and polyether diamine;

b. at least one of the polymers is the reaction product of diisocyanate and a polyetherdiamine to form a pre-polymer, and the reaction product of the pre-polymer and a diol to form a polyether urea urethane where optionally the polyetherdiamine comprises at least one type of oxyalkylene sequence selected from the group consisting of oxyethylene, oxypropylene, oxytrimethylene and oxytetramethylene sequences and/or the polyetherdiamine is a blend of polyetherdiamines and/or the polyetherdiamine is not a blend of polyetherdiamines and/or the polyetherdiamine is a random copolymer of two or more oxyalkylene sequences and/or the polyetherdiamine is a block copolymer of two or more oxyalkylene sequences; the diol is an aliphatic diol; the diol is an aromatic diol; the diol is a polyether diol; the diol is a blend of diols; the diol is a blend of aliphatic diol and polyetherdiol; the diisocyanate is an aliphatic diisocyanate and the reactants do not include an aromatic diisocyanate to form a polyether urea urethane; the diisocyanate is a mixture of aliphatic diisocyanates and the reactants do not include an aromatic diisocyanate to form a polyether urea urethane; the diisocyanate is an aromatic diisocyanate and the reactants do not include an aliphatic diisocyanate to form a polyether urea urethane; the diisocyanate is a mixture of aromatic diisocyanates and the reactants do not include an aliphatic diisocyanate to form a polyether urea urethane; the diisocyanate is a mixture of aromatic diisocyanate and aliphatic diisocyanate to form a polyether urea urethane;

c. at least one of the polymers is the reaction product of a diisocyanate and a diol where optionally the diol is a polyether diol; the polyether diol comprises at least one type of oxyalkylene sequence selected from the group consisting of oxyethylene, oxypropylene, oxytrimethylene and oxytetramethylene sequences; the polyetherdiol is a blend of polyetherdiols; the polyether diol is not a blend of polyetherdiols; the polyetherdiol is a random copolymer of two or more oxyalkylene sequences; the polyetherdiol is a block copolymer of two or more oxyalkylene sequences; the diisocyanate is an aliphatic diisocyanate and the reactants do not include an aromatic diisocyanate; the diisocyanate is a mixture of aliphatic diisocyanates and the reactants do not include an aromatic diisocyanate; the diisocyanate is an aromatic diisocyanate and the reactants do not include an aliphatic diisocyanate; the diisocyanate is a mixture of aromatic diisocyanates and the reactants do not include an aliphatic diisocyanate; the diisocyanate is a mixture of aromatic diisocyanate and aliphatic diisocyanate; diisocyanate and diol are the only reactants; the molar ratio of diisocyanate to polyether diol is in the range of 0.95 to 1.05;

d. at least one of the polymers is the reaction product of a diisocyanate and a diamine where optionally the diamine is a polyether diamine; the polyether diamine comprises at least one type of oxyalkylene sequence selected from the group consisting of oxyethylene, oxypropylene, oxytrimethylene and oxytetramethylene sequences; the polyetherdiamine is a blend of polyetherdiamines; the polyether diamine is not a blend of polyetherdiamines; the polyetherdiamine is a random copolymer of two or more oxyalkylene sequences; the polyetherdiamine is a block copolymer of two or more oxyalkylene sequences; the diisocyanate is an aliphatic diisocyanate and the reactants do not include an aromatic diisocyanate; the diisocyanate is a mixture of aliphatic diisocyanates and the reactants do not include an aromatic diisocyanate; the diisocyanate is an aromatic diisocyanate and the reactants do not include an aliphatic diisocyanate; the diisocyanate is a mixture of aromatic diisocyanates and the reactants do not include an aliphatic diisocyanate; the diisocyanate is a mixture of aromatic diisocyanate and aliphatic diisocyanate; diisocyanate and diamine are the only reactants; the molar ratio of diisocyanate to polyether diamine is in the range of 0.95 to 1.05;

e. at least one of the polymers is the reaction product of a diisocyanate and either (a) a mixture comprising polyether diol and polycarbonate diol or (b) a polyether polycarbonate diol where optionally the polyetherdiol comprises at least one type of oxyalkylene sequence selected from the group consisting of oxyethylene, oxypropylene, oxytrimethylene and oxytetramethylene sequences; the polyetherdiol is a blend of polyetherdiols; the polyether diol is not a blend of polyetherdiols; the polyetherdiol is a random copolymer of two or more oxyalkylene sequences; the polyetherdiol is a block copolymer of two or more oxyalkylene sequences; the polycarbonate diol is poly(hexamethylene carbonate)diol; the polycarbonate diol is poly(ethylene-carbonate)diol; the polycarbonate diol is the reaction product of trimethylene carbonate and a diol; the diisocyanate is an aliphatic diisocyanate and the reactants do not include an aromatic diisocyanate; the diisocyanate is a mixture of aliphatic diisocyanates and the reactants do not include an aromatic diisocyanate; the diisocyanate is an aromatic diisocyanate and the reactants do not include an aliphatic diisocyanate; the diisocyanate is a mixture of aromatic diisocyanates and the reactants do not include an aliphatic diisocyanate; the diisocyanate is a mixture of aromatic diisocyanate and aliphatic diisocyanate; the polymer is further chain extended by reaction with a diol;

f. at least one of the polymers is the reaction product of a diamine and a pre-polymer, where the pre-polymer is the reaction product of a diisocyanate and either (a) a mixture comprising polyether diol and polycarbonate diol or (b) a polyether polycarbonate diol, where optionally the polyetherdiol comprises at least one type of oxyalkylene sequence selected from the group consisting of oxyethylene, oxypropylene, oxytrimethylene and oxytetramethylene sequences; the polyetherdiol is a blend of polyetherdiols; the polyether diol is not a blend of polyetherdiols; the polyetherdiol is a random copolymer of two or more oxyalkylene sequences; the polyetherdiol is a block copolymer of two or more oxyalkylene sequences; the polycarbonate diol is poly(hexamethylene carbonate)diol; the polycarbonate diol is poly(ethylene-carbonate)diol; the polycarbonate diol is the reaction product of trimethylene carbonate and a diol; the diisocyanate is an aliphatic diisocyanate and the reactants do not include an aromatic diisocyanate; the diisocyanate is a mixture of aliphatic diisocyanates and the reactants do not include an aromatic diisocyanate; the diisocyanate is an aromatic diisocyanate and the reactants do not include an aliphatic diisocyanate; the diisocyanate is a mixture of aromatic diisocyanates and the reactants do not include an aliphatic diisocyanate; the diisocyanate is a mixture of aromatic diisocyanate and aliphatic diisocyanate; the diamine is an aliphatic diamine; the diamine is a polyether diamine; the diamine is a blend of diamines; the diamine is a blend of aliphatic diamine and polyether diamine;

g. at least one of the polymers is the reaction product of a diisocyanate and either (a) a mixture comprising polyether diol and polyester diol or (b) a polyether polyester diol, where optionally the polyether diol comprises at least one type of oxyalkylene sequence selected from the group consisting of oxyethylene, oxypropylene, oxytrimethylene and oxytetramethylene sequences; the polyether diol is a blend of polyetherdiols; the polyether diol is not a blend of polyetherdiols; the polyether diol is a random copolymer of two or more oxyalkylene sequences; the polyether diol is a block copolymer of two or more oxyalkylene sequences; the diisocyanate is an aliphatic diisocyanate and the reactants do not include an aromatic diisocyanate; the diisocyanate is a mixture of aliphatic diisocyanates and the reactants do not include an aromatic diisocyanate; the diisocyanate is an aromatic diisocyanate and the reactants do not include an aliphatic diisocyanate; the diisocyanate is a mixture of aromatic diisocyanates and the reactants do not include an aliphatic diisocyanate; the diisocyanate is a mixture of aromatic diisocyanate and aliphatic diisocyanate;

h. at least one of the polymers is the reaction product of a diamine and a pre-polymer, where the pre-polymer is the reaction product of a diisocyanate and either (a) a mixture comprising polyether diol and polyester diol or (b) a polyether polyester diol, where optionally the polyetherdiol comprises at least one type of oxyalkylene sequence selected from the group consisting of oxyethylene, oxypropylene, oxytrimethylene and oxytetramethylene sequences; the polyetherdiol is a blend of polyetherdiols; the polyether diol is not a blend of polyetherdiols; the polyetherdiol is a random copolymer of two or more oxyalkylene sequences; the polyetherdiol is a block copolymer of two or more oxyalkylene sequences; the diisocyanate is an aliphatic diisocyanate and the reactants do not include an aromatic diisocyanate; the diisocyanate is a mixture of aliphatic diisocyanates and the reactants do not include an aromatic diisocyanate; the diisocyanate is an aromatic diisocyanate and the reactants do not include an aliphatic diisocyanate; the diisocyanate is a mixture of aromatic diisocyanates and the reactants do not include an aliphatic diisocyanate; the diisocyanate is a mixture of aromatic diisocyanate and aliphatic diisocyanate; the diamine is an aliphatic diamine; the diamine is a polyether diamine; the diamine is a blend of diamines; the diamine is a blend of aliphatic diamine and polyether diamine;

i. at least one of the polymers is bio-stable;

j. at least one of the polymers absorbs at least 50% of its weight in water when immersed in 1% aqueous methyl cellulose at 37° C. for 16 hours; and

k. at least one of the polymers has a COF of 0.001 to 0.15; at least one of the polymers has an intrinsic viscosity of 3-8 dl/g.

### Methods for Forming Layers and Multilayer Compositions and Desired Shapes

The polymer may be used to form a layer in the form of a film or sheet, or other suitable shape. Suitable shapes may be achieved preferably by dip-coating of a mold into a liquid solution containing the polymer. Dip-coating can be performed through a method involving multiple dips of the mold into a liquid solution of the polymer, wherein the polymer is dissolved in a suitable solvent or mixture of solvents. Each dip will create a part of the center portion of the cap and also part of the rim portion when present. One type of suitable solvent is a fluorinated solvent that is highly volatile, such as trifluorethanol (TFE) or hexafluoroisopropanol (HFIP). Rather than use a fluorinated solvent, the solvent could be a chlorinated solvent, a polar organic solvent, or a mixture of solvents including fluorinated, chlorinated and/or polar organic solvents. Examples of suitable chlorinated and polar organic solvents are chloroform, dichloromethane, isopropanol and acetone. The solvent may be a mixture of fluorinated solvent and a chlorinated solvent, or a mixture of a fluorinated solvent and a polar organic solvent, or a mixture of fluorinated solvent, chlorinated solvent and polar organic solvent, or a mixture of a chlorinated solvent and a polar organic solvent which lacks a fluorinated solvent altogether. The mold is dipped multiple times until the appropriate thickness of the final film is formed on the surface of the mold. Another option for forming a desired shape of PEU is to prepare the PEU within a mold of a desired shape.

In one aspect there is provided a method for forming a multilayer polymeric composition. The method comprises: (a) providing a first solution comprising a first polymer; (b) providing a second solution comprising a second polymer, the first and second polymers being non-identical; (c) providing a support in the shape of a bone or a portion of a bone; (d) depositing the first solution onto the support or portion thereof to provide a first coated support; (e) depositing the first solution onto the first coated support a plurality of times to provide a first layer on the support or portion thereof; (f) depositing the second solution onto the first layer to provide a second coated support; and (g) depositing the second solution onto the second coated support a plurality of times to provide a second layer, the multilayer composition comprising the first layer and the second layer. Optionally, one or more of the following descriptions may be used to further characterize the method and/or the composition made by the method: the support or a coated support is dipped into a first solution or a second solution to provide the depositing; the first solution has a viscosity of 1 cps to 100 k cps, or 1 cps to 50 k cps, or 1 cps to 25 k cps, or 1 cps to 10 k cps, or 1 cps to 5 k cps, or 1 cps to 2.5 k cps, or 5 cps to 2.5 k cps, or 85 cps to 2 k cps, or 85 cps to 2.5 k cps, or 85 cps to 1.5 k cps, or 85 cps to 1 k cps, or 150 cps to 1 k cps; the first solution contains the first polymer at a concentration of 1% to 10% per weight, based on weight of solution; the support or coated support is spun while it is being dipped into the first solution or the second solution; the support or coated support is held static in the first solution or the second solution during the dipping process; the first solution is stirred while the support is dipped in the first solution; the support or coated support is dipped twice in the first solution before allowing solvent to evaporate from the coated support; the first solution and the second solution contain the same solvent(s); the support or coated support is dipped for 1 to 30 seconds in the first solution; the first solution or second solution is sprayed onto the support or the first coated support; the support or coated support is spun while it is being sprayed; the first solution has a viscosity of 1 cps to 100 k cps, or 1 cps to 50 k cps, or 1 cps to 25 k cps, or 1 cps to 10 k cps, or 1 cps to 5 k cps, or 1 cps to 2.5 k cps, or 5 cps to 2.5 k cps, or 85 cps to 2 k cps, or 85 cps to 2.5 k cps, or 85 cps to 1.5 k cps, or 85 cps to 1 k cps, or 150 cps to 1 k cps; the process includes evaporating solvent from a layer of the first solution on the first coated support, prior to deposition of a further layer of the first solution onto the first coated support; the first coated support is dry to the touch when a further layer of first solution is deposited onto the first coated support; the first coated support is tacky when a further layer of first solution is deposited onto the first coated support; the solvent evaporates into an environment having a humidity of 10-85% or 10-70% or 10-55% or 10-35%; the solvent evaporates into an environment having a pressure of less than atmospheric pressure; the solvent evaporates into an environment that is warmer than the temperature of the first solution; the first solution is warmer than room temperature; the first solvent evaporates into an environment that is cooler than the temperature of the first solution; the solvent evaporates for a time of 5 to 120 minutes; the support or coated support is rotated during solvent evaporation; a solution is deposited 5 to 100 times onto the support or coated support to form a layer; the support has spatial dimensions copied from spatial dimensions of a bone of a patient for whom the multilayer polymeric composition is intended; a solution further comprises a pharmaceutically active agent; the method further includes placing the multilayer composition into a solution that comprises a pharmaceutically active agent, and absorbing pharmaceutically active agent into the multilayer composition; the method further includes separating the multilayer composition from the support; the multilayer composition is soaked in an aqueous medium before being separated from the support; the multilayer composition is soaked in a non-aqueous medium before being separated from the support.

In another aspect there is provided another method for forming a multilayer polymeric composition. The method includes: (a) providing a first solution comprising a first polymer; (b) providing a second solution comprising a second polymer, the first and second polymers being non-identical; (c) providing a support in the shape of a bone or a portion of a bone; (d) depositing the first solution onto a portion of the support to provide a first coated support; (e) depositing the first solution onto the first coated support a plurality of times to provide a first layer on the portion of the support; (f) removing the first layer from the support; (g) inverting the first layer to provide an inverted first layer, and placing the inverted first layer onto the support to provide an inverted first support coating; (h) depositing the second solution onto the inverted first support coating to provide a second coated support; and (i) depositing the second solution onto the second coated support a plurality of times to provide the a second layer, the multilayer composition comprising the first layer and the second layer. Optionally, one or more of the following descriptions may be used to further characterize the method and/or the composition made by the method: the support or a coated support is dipped into a first solution or a second solution to provide the depositing; the first solution has a viscosity of 1 1 cps to 100 k cps, or 1 cps to 50 k cps, or 1 cps to 25 k cps, or 1 cps to 10 k cps, or 1 cps to 5 k cps, or 1 cps to 2.5 k cps, or 5 cps to 2.5 k cps, or 85 cps to 2 k cps, or 85 cps to 2.5 k cps, or 85 cps to 1.5 k cps, or 85 cps to 1 k cps, or 150 cps to 1 k cps; the first solution contains the first polymer at a concentration of 1% to 10% per weight, based on weight of solution; the support or coated support is spun while it is being dipped into the first solution or the second solution; the support or coated support is held static in the first solution or the second solution during the dipping process; the first solution is stirred while the support is dipped in the first solution; the support or coated support is dipped twice in the first solution before allowing solvent to evaporate from the coated support; the first solution and the second solution contain the same solvent(s); the support or coated support is dipped for 1 to 30 seconds in the first solution; the first solution or second solution is sprayed onto the support or the first coated support; the support or coated support is spun while it is being sprayed; the first solution has a viscosity of 1 cps to 100 k cps, or 1 cps to 50 k cps, or 1 cps to 25 k cps, or 1 cps to 10 k cps, or 1 cps to 5 k cps, or 1 cps to 2.5 k cps, or 5 cps to 2.5 k cps, or 85 cps to 2 k cps, or 85 cps to 2.5 k cps, or 85 cps to 1.5 k cps, or 85 cps to 1 k cps, or 150 cps to 1 k cps; the method further includes evaporating solvent from a layer of the first solution on the first coated support, prior to deposition of a further layer of the first solution onto the first coated support; the first coated support is dry to the touch when a further layer of first solution is deposited onto the first coated support; the first coated support is tacky when a further layer of first solution is deposited onto the first coated support; the solvent evaporates into an environment having a humidity of 10-85% or 10-70% or 10-55% or 10-35%; the solvent evaporates into an environment having a pressure of less than atmospheric pressure; the solvent evaporates into an environment that is warmer than the temperature of the first solution; the solvent evaporates for a time of 5 to 120 minutes; the support or coated support is rotated during solvent evaporation; a solution is deposited 5 to 100 times onto the support or coated support to form a layer; the support has spatial dimensions copied from spatial dimensions of a bone of a patient for whom the multilayer polymeric composition is intended; a solution further comprises a pharmaceutically active agent; the method further includes placing the multilayer composition into a solution that comprises a pharmaceutically active agent, and absorbing pharmaceutically active agent into the multilayer composition; the method further includes separating the multilayer composition from the support; the first layer and/or the multilayer composition is soaked in an aqueous medium before being separated from the support; the first layer and/or the multilayer composition is soaked in a non-aqueous medium before being separated from the support.

In another aspect there is provided another method for forming a multilayer polymeric composition. The method includes: (a) providing a first solution comprising a first polymer; (b) providing a second solution comprising a pre-polymer of a second polymer, the first and second polymers being non-identical; (c) providing a third solution comprising a reactant reactive with the pre-polymer, the reactant and the pre-polymer being reactive to form the second polymer; (d) providing a fourth solution comprising a third polymer; (e) providing a support in the shape of a bone or a portion thereof; (f) depositing the first solution onto the support or portion thereof to provide a first coated support; (g) depositing the first solution onto the first coated support a plurality of times to provide a first layer; (h) depositing the second solution on the first layer to provide a second coated support; (i) depositing the third solution on the second coated support to form an intermediate layer and provide a third coated support; (j) depositing the fourth solution onto the third coated support to provide a fourth coated support; and (k) depositing the fourth solution on the fourth coated support a plurality of times to provide a second layer, the multilayer composition comprising the first layer, the intermediate layer and the second layer. Optionally, one or more of the following descriptions may be used to further characterize the method and/or the composition made by the method: the prepolymer comprises urethane groups and isocyanate end groups; the reactant comprises amine end groups; the reactant is a pre-polymer comprising urethane groups, carbonate groups, and amine end groups; the prepolymer comprises urethane groups, carbonate groups, and amine end groups; the reactant comprises isocyanate groups; the reactant is a pre-polymer comprising urethane groups and isocyanate end groups; the support or a coated support is dipped into a solution to provide the depositing; a solution has a viscosity of 1 cps to 100 k cps, or 1 cps to 50 k cps, or 1 cps to 25 k cps, or 1 cps to 10 k cps, or 1 cps to 5 k cps, or 1 cps to 2.5 k cps, or 5 cps to 2.5 k cps, or 85 cps to 2 k cps, or 85 cps to 2.5 k cps, or 85 cps to 1.5 k cps, or 85 cps to 1 k cps, or 150 cps to 1 k cps; the first solution contains the first polymer at a concentration of 1% to 10% per weight, based on weight of solution; the support or coated support is spun while it is being dipped into a solution; the support or coated support is held static in a solution during the dipping process; a solution is stirred while the support is dipped in the solution; the support or coated support is dipped twice in a solution before allowing solvent to evaporate from the coated support; the first solution and the second solution contain the same solvent(s); the support or coated support is dipped for 1 to 30 seconds a solution; a solution is sprayed onto the support or the coated support; the support or coated support is spun while it is being sprayed; a solution has a viscosity of 1 cps to 100 k cps, or 1 cps to 50 k cps, or 1 cps to 25 k cps, or 1 cps to 10 k cps, or 1 cps to 5 k cps, or 1 cps to 2.5 k cps, or 5 cps to 2.5 k cps, or 85 cps to 2 k cps, or 85 cps to 2.5 k cps, or 85 cps to 1.5 k cps, or 85 cps to 1 k cps, or 150 cps to 1 k cps; the method further includes evaporating solvent from the first solution on the first coated support, prior to deposition of further first solution onto the first coated support; the first coated support is dry to the touch when further first solution is deposited onto the first coated support; the first coated support is tacky when further first solution is deposited onto the first coated support; the solvent evaporates into an environment having a relative humidity 10-85% or 10-70% or 10-55% or 10-35%; the solvent evaporates into an environment having a pressure of less than atmospheric pressure; the solvent evaporates into an environment that is warmer than the temperature of the first solution; the solvent evaporates for a time of 5 to 120 minutes; the support or coated support is rotated during solvent evaporation; a solution is deposited 5 to 100 times onto the support or coated support to form a layer; the support has spatial dimensions copied from spatial dimensions of a bone of a patient for whom the multilayer polymeric composition is intended; a solution further comprises a pharmaceutically active agent; the method further includes placing the multilayer composition into a solution that comprises a pharmaceutically active agent, and absorbing pharmaceutically active agent into the multilayer composition; the method further includes separating the multilayer composition from the support; the multilayer composition is soaked in an aqueous medium before being separated from the support; the multilayer composition is soaked in a non-aqueous medium before being separated from the support.

Optionally, in any of the multilayer compositions and cap described herein, the innermost layer can be formed by co-electrospinning a PEUU and a porogen, such as polyethylene glycol, or porogen fibers fabricated from PEEUU or absorbable, biocompatible, aliphatic polyester for the tailored ingrowth of bone or cartilage. The porogen diffuses out of the layer or degrades to create a porous microstructure allowing for cellular ingrowth to provide improved fixation of the femoral cap to the femoral head; in optional embodiments, growth factors that promote osteogenesis and cartilage growth are incorporated within the innermost layer to facilitate the ingrowth of chrondrocytes and/or osteocytes. Suitable pore sizes range from 100-500 micrometers in diameter to allow for cellular ingrowth. Suitable fiber diameters preferably range from 0.1 to 10 micrometers and are preferably greater than 1.0 micrometers.

### Kits

Within certain additional aspects of the invention, kits are provided comprising a multilayer composition, e.g., a cap, which has been designed for a joint (e.g., a femoral head), and one or more additional components. For example, a suitable additional component is an auxiliary composition for injection or administration into the joint at the time of surgery, or during subsequent rounds of administration post-surgery. Within certain preferred embodiments the auxiliary composition for injection or administration into the joint comprises an anesthetic and an anti-inflammatory agent, and optionally, an antibacterial agent. Particularly preferred polymers that are present in the multilayer composition are PEU polymers, which include, for example, PEUT, PEUA, PEUU, PECUT, PECUA, PECUU, PEEUT, PEEUA, and PEEUU.

In one aspect there is provided a kit, where the kit includes a multilayer composition as described herein. For example the multilayer composition may comprise, or be prepared by a method that comprises:

a. a first layer in contact with a second layer, the first layer comprising a first polymer and the second layer comprising a second polymer. The multilayer composition is advantageously used in an environment where the first layer is exposed to a different environmental condition than is the second layer. For this reason, the polymers of the first and second layers, i.e., the first and second polymers, are non-identical and are selected to provide different properties;

b. a center and a rim, where the rim surrounds the center. At least one of the center and the rim comprises a first layer in contact with a second layer, the first layer comprising a first polymer and the second layer comprising a second polymer. Optionally, in each of the cap embodiments described herein, the rim is not present, but instead the cap terminates with the center portion. Optionally, in each of the cap embodiments described herein, the center portion and the rim (when present) are formed from the same first and second layers, i.e., the innermost layer of the rim and the innermost layer of the center portion are identical and contiguous, while the outermost layer of the rim and the outermost layer of the center portion are identical and contiguous;

c. a cap in combination with a support. The support may be a bone, including a portion of a bone, or a non-bone material that is in the shape of a bone, again including a portion of a shape of a bone. The cap should fit snugly around a portion of the support. The cap comprises a multilayer composition comprising a first layer in contact with a second layer, the first layer comprising a first polymer and the second layer comprising a second polymer;

d. (a) providing a first solution comprising a first polymer; (b) providing a second solution comprising a second polymer, the first and second polymers being non-identical; (c) providing a support in the shape of a bone or a portion of a bone; (d) depositing first solution onto the support or portion thereof to provide a first coated support; (e) depositing additional first solution onto the first coated support a plurality of times to provide a first layer on the support or portion thereof; (f) depositing second solution onto the first layer to provide a second coated support; and (g) depositing additional second solution onto the second coated support a plurality of times to provide a second layer, the multilayer composition comprising the first layer and the second layer;

e. (a) providing a first solution comprising a first polymer; (b) providing a second solution comprising a second polymer, the first and second polymers being non-identical; (c) providing a support in the shape of a bone or a portion of a bone; (d) depositing the first solution onto a portion of the support to provide a first coated support; (e) depositing the first solution onto the first coated support a plurality of times to provide a first layer on the portion of the support; (f) removing the first layer from the support; (g) inverting the first layer to provide an inverted first layer, and placing the inverted first layer onto the support to provide an inverted first support coating; (h) depositing the second solution onto the inverted first support coating to provide a second coated support; and (i) depositing the second solution onto the second coated support a plurality of times to provide a second layer, the multilayer composition comprising the first layer and the second layer;

f. (a) providing a first solution comprising a first polymer; (b) providing a second solution comprising a pre-polymer of a second polymer, the first and second polymers being non-identical; (c) providing a third solution comprising a reactant reactive with the pre-polymer, the reactant and the pre-polymer being reactive to form the second polymer; (d) providing a fourth solution comprising a third polymer; (e) providing a support in the shape of a bone or a portion thereof; (f) depositing the first solution onto the support or portion thereof to provide a first coated support; (g) depositing the first solution onto the first coated support a plurality of times to provide a first layer; (h) depositing the second solution on the first layer to provide a second coated support; (i) depositing the third solution on the second coated support to form an intermediate layer and provide a third coated support; (j) depositing the fourth solution onto the third coated support to provide a fourth coated support; and (k) depositing the fourth solution on the fourth coated support a plurality of times to provide a second layer, the multilayer composition comprising the first layer, the intermediate layer and the second layer.

In addition to the multilayer composition, the kit will include one or more additional components. In one aspect, the additional component is an anesthetic.

### PEU General Chemical Description and Preparation

Optionally, in each of the compositions and methods and kits disclosed herein, the first, second, third, etc. polymer may be a polymeric material referred to herein as PEU. The PEU is a polymeric material that includes a plurality of linking groups selected from urea and urethane groups. The PEU will additionally include a plurality of segments located between adjacent linking groups. In other words, the PEU may be described in whole or part as having portions described as urethane-segment-urethane and/or urea-segment-urea and/or urea-segment-urethane which may also be written as urethane-segment-urea. At least some of the segments of the PEU are polyoxyalkylene. Other exemplary segments include hydrocarbons, polyesters, polycarbonates and polysiloxanes. The segments that do not comprise entirely hydrocarbon will comprise some hydrocarbon located between adjacent functional groups. For example, hydrocarbon will be located between adjacent ester groups of a polyester, between adjacent ether groups of a polyether, between adjacent siloxane groups of a polysiloxane, and between adjacent carbonate groups of a polycarbonate.

The polyoxyalkylene segments have alkylene groups between adjacent oxygen atoms. The polyoxyalkylene segments will contain at least one type of oxyalkylene sequence selected from the group consisting of oxyethylene, oxypropylene, oxytrimethylene, and oxytetramethylene sequences.

A segment may contain entirely hydrocarbon, and the hydrocarbon may be aliphatic or aromatic. In the case of an aliphatic hydrocarbon, the number of carbon atoms in the hydrocarbon may vary between 1 and about 12. When the hydrocarbon is an aromatic hydrocarbon, it will contain at least 6 carbons and may contain as many as about 16 carbons. In various specific aspects, the hydrocarbon contains 2 or 3 or 4 or 5 or 6 or 7 or 8 or 9 or 10 carbon atoms. When hydrocarbon is between two functional groups, e.g., between two ester or two carbonate groups, in various aspects the hydrocarbon as 2 or 3 or 4 or 5 or 6 or more carbons. The hydrocarbon may be saturated.

Exemplary PEUs include polyether urethane (PEUT), polyether urea (PEUA), polyether urea urethane (PEUU), polyether carbonate urethane (PECUT), polyether carbonate urea (PECUA), polyether carbonate urethane urea (PECUU), polyether ester urethane (PEEUT), polyether ester urea (PEEUA), and polyether ester urethane urea (PEEUU).

### Reactants

The PEU may be prepared by reacting together various di-functional reactants to form linking groups, the linking groups being formed from a functional group of a first reactant reacting with a functional group of a second reactant.

A diisocyanate may be used as a reactant to form the PEU. As is well known, the reaction between a hydroxyl (alcohol) group and an isocyanate group will provide a urethane group, while the reaction between an amine group and an isocyanate group will provide a urea group. Exemplary aliphatic diisocyanates include tetramethylene diisocyanate, hexamethylene diisocyanate, octamethylene diisocyanate, decamethylene diisocyanate, dodecamethylene diisocyanate, and cyclohexane bis-(methylene isocyanate). Aromatic diisocyanates may additionally, or alternatively, be used as a reactant to form the PEU.

A polyether diol may be used as a reactant to form the PEU. The polyether diol will introduce polyoxyalkylene segments, in other words polyether segments, into the PEU. The polyether diol may comprise a homopolymer of oxyalkylene groups, or a copolymer of two different oxyalkylene groups. The copolymer of two different oxyalkyelene groups may be a random or block copolymer, for example, a diblock copolymer, or a triblock copolymer. The copolymer may be an alternating copolymer of two or more oxyalkylene sequences, or may be an alternating copolymer of two different oxyalkylene repeating units. Exemplary oxyalkylene moieties include oxyethylene, oxypropylene, oxytrimethylene, and oxytetramethylene.

A polyether diamine may be used as a reactant to form the PEU. When a polyether diamine is reacted with a diisocyanate-containing reactant, the result will be a polyether urea moiety. The polyether diamine may comprise a homopolymer of oxyalkylene groups, or a copolymer of two different oxyalkylene groups. The copolymer may be a random or block copolymer, for example, a diblock copolymer, or a triblock copolymer. The copolymer may be an alternating copolymer of two or more oxyalkylene sequences. The copolymer may be an alternating copolymer of two different oxyalkylene repeat units. Exemplary oxyalkylene moieties include oxyethylene, oxypropylene, oxytrimethylene, and oxytetramethylene.

An aliphatic diol may be used as a reactant to form the PEU. Exemplary alkylene groups include ethylene, propylene (branched or straight chain), butylene (branched or straight chain), hexylene (branched, straight chain or cyclic) and octylene (branched, straight chain, or cyclic).

An aliphatic diamine may be used as a reactant to form the PEU. Exemplary alkylene groups include ethylene, propylene (branched or straight chain), butylene (branched or straight chain), hexylene (branched, straight chain or cyclic) and octylene (branched, straight chain, or cyclic).

An aromatic diol may be used as a reactant to form the PEU. Examples include catechol, resorcinol, hydroquinone and the reactions products thereof, for example, the reaction product of reaction products of resorcinol and ethylene carbonate. Other aromatic diol include bisphenol A and 4,4′-dihydroxybiphenyl.

An aromatic diamine may be used as a reactant to form the PEU. Examples include 1,2-diaminobenzene, 1,3-diaminobenzene, 1,4-diaminobenzene, toluene diamine (e.g., 1,2-diamino-3-methylbenzene, 1,2-diamino-4-methylbenzene, 1,3-diamino-2-methylbenzene, 1,3-diaminoe-4-methylbenzene, 1,4-diamino-2-methylbenzene, 1,4-diamino-3-methylbenzene), alkyl-substituted toluenediamine (e.g., 3,5-diethyltoluene-2,4-diamine and 3,5-diethyltoluene-2,6-diamine), and p-xylyenediamine.

A carbonate may be used as a reactant to form the PEU. Examples include trimethylene carbonate, poly(hexamethylene carbonate)diol, poly(ethylene-carbonate)diol, poly(propylene-carbonate)diol, and poly(butylene-carbonate)diol. When hydroxyl groups are located at either end of a polycarbonate, the material will be referred to herein as a polycarbonate diol.

Glycolide or substituted glycolide may be used as a reactant to form the PEU. The inclusion of glycolide or substituted glycolide among the reactants can achieve formation of ester groups in the PEU. Exemplary substituted glycolides include methyl glycolide (also known as lactide), ethyl glycolide, hexyl glycolide, and isobutyl glycolide.

A blend of diol or a blend of diamine may be two or more of aliphatic diol (or diamine), aromatic diol (or diamine), and polyether diol (or diamine).

### Specific PEU Embodiments

Depending on the selection of reactants, the PEU will have various linkage groups and segments.

In one embodiment, the PEU is a polyether urethane (PEUT), i.e., a polymer that contains only urethane as the linking group, and additionally contains polyether segments. The PEUT may be prepared by reaction of a diisocyanate and a polyether diol. Alternatively, the PEUT may be prepared from a polyetherdiisocyanate and an aliphatic diol, e.g., ethylene glycol. The molar ratio of diisocyanate to polyether diol will typically range from 0.95 to 1.05, where the preferred stoichiometric ratio is as close to 1:1 as possible in order to attain the highest molecular weight polymer. Similarly, the molar ratio of polyetherdiisocyanate and aliphatic diol will typically fall within the range of 0.95 to 1.05, and have a preferred stoichiometric ratio of 1:1 in order to attain high molecular weight polymer. The following numbered embodiments provide exemplary PEUT:
1. A polymer composition which is the reaction product of a diisocyanate and a diol.
2. The polymer of embodiment 1 wherein the diol is a polyether diol.
3. The polymer of embodiment 2 wherein the polyether diol comprises at least one type of oxyalkylene sequence selected from the group consisting of oxyethylene, oxypropylene, oxytrimethylene and oxytetramethylene sequences.
4. The polymer of embodiments 1 or 2 wherein the polyetherdiol is a blend of polyetherdiols.
5. The polymer of embodiments 1 or 2 wherein the polyetherdiol is not a blend of polyetherdiols.
6. The polymer of any of embodiments 2-5 wherein the polyetherdiol is a random copolymer of two or more oxyalkylene sequences.
7. The polymer of any of embodiments 2-5 wherein the polyetherdiol is a block copolymer of two or more oxyalkylene sequences, where in optional embodiments the block copolymer is an alternating copolymer of two or more oxyalkylene sequences or is an alternating copolymer of two different oxyalkylene repeat units.
8. The polymer of any of embodiments 1-7 wherein the diisocyanate is an aliphatic diisocyanate and the reactants do not include an aromatic diisocyanate.
9. The polymer of any of embodiments 1-7 wherein the diisocyanate is a mixture of aliphatic diisocyanates and the reactants do not include an aromatic diisocyanate.
10. The polymer of any of embodiments 1-7 wherein the diisocyanate is an aromatic diisocyanate and the reactants do not include an aliphatic diisocyanate.
11. The polymer of any of embodiments 1-7 wherein the diisocyanate is a mixture of aromatic diisocyanates and the reactants do not include an aliphatic diisocyanate.
12. The polymer of any of embodiments 1-7 wherein the diisocyanate is a mixture of aromatic diisocyanate and aliphatic diisocyanate.
13. The polymer of any of embodiments 1-12 wherein diisocyanate and diol are the only reactants.
14. The polymer of any of embodiments 2-13 wherein the molar ratio of diisocyanate to polyether diol is in the range of 0.95 to 1.05.

In one embodiment, the PEU is a polyether urea (PEUA), i.e., a polymer that contains only urea as the linking group, and additionally contains polyether segments. The PEUA may be prepared by reaction of a diisocyanate and a polyether diamine. Alternatively, the PEUT may be prepared by reaction of a polyether diisocyanate and an aliphatic diamine, e.g., ethylene diamine. The molar ratio of diisocyanate to polyether diamine will typically range from 0.95 to 1.05, where the preferred stoichiometric ratio is as close to 1:1 as possible in order to attain the highest molecular weight polymer. The following numbered embodiments provide exemplary PEUA:
1. A polymer composition which is the reaction product of a diisocyanate and a diamine.
2. The polymer of embodiment 1 wherein the diamine is a polyether diamine.
3. The polymer of embodiment 2 wherein the polyether diamine comprises at least one type of oxyalkylene sequence selected from the group consisting of oxyethylene, oxypropylene, oxytrimethylene and oxytetramethylene sequences.
4. The polymer of embodiments 1 or 2 wherein the polyetherdiamine is a blend of polyetherdiamines.
5. The polymer of embodiments 1 or 2 wherein the polyether diamine is not a blend of polyetherdiamines.
6. The polymer of any of embodiments 2-5 wherein the polyetherdiamine is a random copolymer of two or more oxyalkylene sequences.
7. The polymer of any of embodiments 2-5 wherein the polyetherdiamine is a block copolymer of two or more oxyalkylene sequences, where in optional embodiments the block copolymer is an alternating copolymer of two or more oxyalkylene sequences or is an alternating copolymer of two different oxyalkylene repeat units.
8. The polymer of any of embodiments 1-7 wherein the diisocyanate is an aliphatic diisocyanate and the reactants do not include an aromatic diisocyanate.
9. The polymer of any of embodiments 1-7 wherein the diisocyanate is a mixture of aliphatic diisocyanates and the reactants do not include an aromatic diisocyanate.
10. The polymer of any of embodiments 1-7 wherein the diisocyanate is an aromatic diisocyanate and the reactants do not include an aliphatic diisocyanate.
11. The polymer of any of embodiments 1-7 wherein the diisocyanate is a mixture of aromatic diisocyanates and the reactants do not include an aliphatic diisocyanate.
12. The polymer of any of embodiments 1-7 wherein the diisocyanate is a mixture of aromatic diisocyanate and aliphatic diisocyanate.
13. The polymer of any of embodiments 1-12 wherein diisocyanate and diamine are the only reactants.
14. The polymer of any of embodiments 2-13 wherein the molar ratio of diisocyanate to polyether diamine is in the range of 0.95 to 1.05.

In one embodiment, the PEU is a polyether urea urethane (PEUU), i.e., a polymer that contains both urea and urethane as the only linking groups, and additionally contains polyether segments. The PEUU may be prepared by forming a pre-polymer and then reacting the pre-polymer with either diamine or diol or a mixture thereof. For example, a pre-polymer may be prepared by reacting diisocyanate with polyetherdiol to form a polyether urethane (urethane linkages and polyether segments), and then this pre-polymer is reacted with diamine, e.g., aliphatic diamine, to additionally provide urea linkages and aliphatic segments. Alternatively, a pre-polymer may be prepared by reacting diisocyanate with polyether diamine to form urea linkages and polyether segments with flanking isocyanate end groups, and then this pre-polymer is reacted with diol, e.g., aliphatic diol, to provide urethane linkages and aliphatic segments. A mixture of diol and diamine can also be used, although it should be kept in mind that in most instances the diamine will react more quickly than the diol. The preferred molar ratio of diisocyanate to polyether diol should be about 3:2 for forming the pre-polymer, and the preferred molar ratio of diisocyanate to diamine or diol in the second reaction step is 3:1. Other possible ratios are 2:1 (2:1), 4:3 (4:1), 5:4 (5:1), 6:5 (6:1), 7:6 (7:1), 8:7 (8:1), 9:8 (9:1), and 10:9 (10:1), where the first ratio listed is for diisocyanate to polyether diol in preparation of pre-polymer, and the second ratio in parentheses is the corresponding molar ratio of diisocyanate to diamine or diol for the second reaction step. The following numbered embodiments provide exemplary PEUU:
1. A polymer composition which is the reaction product of a pre-polymer and a diamine, where the pre-polymer is the reaction product of a diisocyanate and a polyetherdiol.
2. The polymer of embodiment 1 wherein the polyetherdiol comprises at least one type of oxyalkylene sequence selected from the group consisting of oxyethylene, oxypropylene, oxytrimethylene and oxytetramethylene sequences.
3. The polymer of embodiments 1 or 2 wherein the polyetherdiol is a blend of polyetherdiols.
4. The polymer of embodiments 1 or 2 wherein the polyether diol is not a blend of polyetherdiols.
5. The polymer of any one of embodiments 1-4 wherein the polyetherdiol is a random copolymer of two or more oxyalkylene sequences.
6. The polymer of any one of embodiments 1-4 wherein the polyetherdiol is a block copolymer of two or more oxyalkylene sequences, where in optional embodiments the block copolymer is an alternating copolymer of two or more oxyalkylene sequences or is an alternating copolymer of two different oxyalkylene repeat units.
7. The polymer of any one of embodiments 1-6 wherein the diamine is an aliphatic diamine.
8. The polymer of any one of embodiments 1-6 wherein the diamine is a polyether diamine.
9. The polymer of any one of embodiments 1-6 wherein the diamine is a blend of diamines.
10. The polymer of any one of embodiments 1-6 wherein the diamine is a blend of aliphatic diamine and polyether diamine.
11. The polymer of any one of embodiments 1-6 wherein the diamine is a blend of aromatic diamine and polyether diamine.
12. The polymer of any one of embodiments 1-6 wherein the diamine is a blend of aromatic diamine and aliphatic diamine.
13. A polymer composition which is the reaction product of a diisocyanate and a polyetherdiamine to form a pre-polymer, and the reaction product of the pre-polymer and a diol to form a polyether urea urethane.
14. The polymer of embodiment 13 wherein the polyetherdiamine comprises at least one type of oxyalkylene sequence selected from the group consisting of oxyethylene, oxypropylene, oxytrimethylene and oxytetramethylene sequences.
15. The polymer of embodiments 13 or 14 wherein the polyetherdiamine is a blend of polyetherdiamines.
16. The polymer of embodiments 13 or 14 wherein the polyetherdiamine is not a blend of polyetherdiamines.
17. The polymer of any one of embodiments 13-16 wherein the polyetherdiamine is a random copolymer of two or more oxyalkylene sequences.
18. The polymer of any one of embodiments 13-16 wherein the polyetherdiamine is a block copolymer of two or more oxyalkylene sequences, where in optional embodiments the block copolymer is an alternating copolymer of two or more oxyalkylene sequences or is an alternating copolymer of two different oxyalkylene repeat units.
19. The polymer of any one of embodiments 13-18 wherein the diol is an aliphatic diol.
20. The polymer of any one of embodiments 13-18 wherein the diol is an aromatic diol.
21. The polymer of any one of embodiments 13-18 wherein the diol is a polyether diol.
22. The polymer of any one of embodiments 13-18 wherein the diol is a blend of diols.
23. The polymer of any one of embodiments 13-18 wherein the diol is a blend of aliphatic diol and polyetherdiol.
24. The polymer of any one of embodiments 13-18 wherein the diol is a blend of aromatic diol and polyether diol.
25. The polymer of any one of embodiments 13-18 wherein the diol is a blend of aromatic diol and aliphatic diol.
26. The polymer of any one of embodiments 1-25 wherein the diisocyanate is an aliphatic diisocyanate and the reactants do not include an aromatic diisocyanate to form a polyether urea urethane.
27. The polymer of any one of embodiments 1-25 wherein the diisocyanate is a mixture of aliphatic diisocyanates and the reactants do not include an aromatic diisocyanate to form a polyether urea urethane.
28. The polymer of any one of embodiments 1-25 wherein the diisocyanate is an aromatic diisocyanate and the reactants do not include an aliphatic diisocyanate to form a polyether urea urethane.
29. The polymer of any one of embodiments 1-25 wherein the diisocyanate is a mixture of aromatic diisocyanates and the reactants do not include an aliphatic diisocyanate to form a polyether urea urethane.
30. The polymer of any one of embodiments 1-25 wherein the diisocyanate is a mixture of aromatic diisocyanate and aliphatic diisocyanate to form a polyether urea urethane.

In one embodiment, the PEU is a polyether carbonate urethane (PECUT), i.e., a polymer that contains urethane linkages, and between the urethane linkages are located a plurality of oxyalkylene groups and a plurality of carbonate groups. In one embodiment, there are two poly(carbonate) groups located between adjacent urethane linkages, where a polyether segment is located between two adjacent poly(carbonate) groups. In one aspect, the weight percent of the combined polyether and polycarbonate segments is 50-99% polycarbonate, or 55-90% polycarbonate, or 60-85% polycarbonate, or 65-75% polycarbonate. The PECUT may be prepared by reacting together a polyether polycarbonate diol, i.e., a diol having a plurality of internal oxyalkyene groups and a plurality of internal carbonate groups, with a diisocyanate. Alternatively, the PECUT may be prepared by reacting together a polyether diol and a polycarbonate diol with a diisocyanate. In either case, the resulting product may be subjected to chain extension with a diol to introduce additional urethane groups. The following numbered embodiments provide exemplary PECUT:
1. A polymer composition which is the reaction product of a diisocyanate and either (a) a mixture comprising polyether diol and polycarbonate diol or (b) a polyether polycarbonate diol.
2. The polymer of embodiment 1 wherein the polyetherdiol comprises at least one type of oxyalkylene sequence selected from the group consisting of oxyethylene, oxypropylene, oxytrimethylene and oxytetramethylene sequences.
3. The polymer of embodiments 1 or 2 wherein the polyetherdiol is a blend of polyetherdiols.
4. The polymer of embodiments 1 or 2 wherein the polyether diol is not a blend of polyetherdiols.
5. The polymer of any one of embodiments 1-4 wherein the polyetherdiol is a random copolymer of two or more oxyalkylene sequences.
6. The polymer of any one of embodiments 1-4 wherein the polyetherdiol is a block copolymer of two or more oxyalkylene sequences, where in optional embodiments the block copolymer is an alternating copolymer of two or more oxyalkylene sequences or is an alternating copolymer of two different oxyalkylene repeat units.
7. The polymer of any one of embodiments 1-6 wherein the polycarbonate diol is poly(hexamethylene carbonate)diol.
8. The polymer of any one of embodiments 1-6 wherein the polycarbonate diol is poly(ethylene-carbonate)diol.
9. The polymer of any one of embodiments 1-6 wherein the polycarbonate diol is the reaction product of trimethylene carbonate and a diol.
10. The polymer of any one of embodiments 1-9 wherein the diisocyanate is an aliphatic diisocyanate and the reactants do not include an aromatic diisocyanate.
11. The polymer of any one of embodiments 1-9 wherein the diisocyanate is a mixture of aliphatic diisocyanates and the reactants do not include an aromatic diisocyanate.
12. The polymer of any one of embodiments 1-9 wherein the diisocyanate is an aromatic diisocyanate and the reactants do not include an aliphatic diisocyanate.
13. The polymer of any one of embodiments 1-9 wherein the diisocyanate is a mixture of aromatic diisocyanates and the reactants do not include an aliphatic diisocyanate.
14. The polymer of any one of embodiments 1-9 wherein the diisocyanate is a mixture of aromatic diisocyanate and aliphatic diisocyanate.
15. The polymer of any one of embodiments 1-14 which is further chain extended by reaction with a diol.

In one embodiment, the PEU is a polyether carbonate urethane urea (PECUU), i.e., a polymer that contains urethane linkages as well as urea linkages, and between the urethane linkages are located a plurality of oxyalkylene groups and a plurality of carbonate groups. In one embodiment, there are two poly(carbonate) groups located between adjacent urethane linkages, where a polyether segment is located between two adjacent poly(carbonate) groups. In one aspect, the weight percent of the combined polyether and polycarbonate segments is 50-99% polycarbonate, or 55-90% polycarbonate, or 60-85% polycarbonate, or 65-75% polycarbonate. The PECUU may be prepared by reacting together a polyether polycarbonate diol, i.e., a diol having a plurality of internal oxyalkyene groups and a plurality of internal carbonate groups, with a diisocyanate. Alternatively, the PECUT may be prepared by reacting together a polyether diol and a polycarbonate diol with a diisocyanate. In either case, the resulting product is subjected to chain extension with a diamine to introduce urea groups. The following numbered embodiments provide exemplary PECUU:
1. A polymer composition which is the reaction product of a diamine and a pre-polymer, where the pre-polymer is the reaction product of a diisocyanate and either (a) a mixture comprising polyether diol and polycarbonate diol or (b) a polyether polycarbonate diol.
2. The polymer of embodiment 1 wherein the polyetherdiol comprises at least one type of oxyalkylene sequence selected from the group consisting of oxyethylene, oxypropylene, oxytrimethylene and oxytetramethylene sequences.
3. The polymer of embodiments 1 or 2 wherein the polyetherdiol is a blend of polyetherdiols.
4. The polymer of embodiments 1 or 2 wherein the polyether diol is not a blend of polyetherdiols.
5. The polymer of any one of embodiments 1-4 wherein the polyetherdiol is a random copolymer of two or more oxyalkylene sequences.
6. The polymer of any one of embodiments 1-4 wherein the polyetherdiol is a block copolymer of two or more oxyalkylene sequences, where in optional embodiments the block copolymer is an alternating copolymer of two or more oxyalkylene sequences or is an alternating copolymer of two different oxyalkylene repeat units.
7. The polymer of any one of embodiments 1-6 wherein the polycarbonate diol is poly(hexamethylene carbonate)diol.
8. The polymer of any one of embodiments 1-6 wherein the polycarbonate diol is poly(ethylene-carbonate)diol.
9. The polymer of any one of embodiments 1-6 wherein the polycarbonate diol is the reaction product of trimethylene carbonate and a diol.
10. The polymer of any one of embodiments 1-9 wherein the diisocyanate is an aliphatic diisocyanate and the reactants do not include an aromatic diisocyanate.
11. The polymer of any one of embodiments 1-9 wherein the diisocyanate is a mixture of aliphatic diisocyanates and the reactants do not include an aromatic diisocyanate.
12. The polymer of any one of embodiments 1-9 wherein the diisocyanate is an aromatic diisocyanate and the reactants do not include an aliphatic diisocyanate.
13. The polymer of any one of embodiments 1-9 wherein the diisocyanate is a mixture of aromatic diisocyanates and the reactants do not include an aliphatic diisocyanate.
14. The polymer of any one of embodiments 1-9 wherein the diisocyanate is a mixture of aromatic diisocyanate and aliphatic diisocyanate.
15. The polymer of any one of embodiments 1-14 wherein the diamine is an aliphatic diamine.
16. The polymer of any one of embodiments 1-14 wherein the diamine is a polyether diamine.
17. The polymer of any one of embodiments 1-14 wherein the diamine is a blend of diamines
18. The polymer of any one of embodiments 1-14 wherein the diamine is a blend of aliphatic diamine and polyether diamine.

In one embodiment, the PEU is a polyether ester urethane (PEEUT), i.e., a polymer that contains urethane linkages, and between the urethane linkages are located polyether and polyester groups. In one embodiment, a single block of polyether which is flanked on either side by a block of polyester is located between urethane linkages. The PEEUT may be prepared by forming a pre-polymer of polyether and polyester having flanking hydroxyl groups. The pre-polymer is then reacted with diisocyanate to form urethane linkages on either side of the polyether polyester diblock. The following numbered embodiments provide exemplary PEEUT:
1. A polymer composition which is the reaction product of a diisocyanate and either (a) a mixture comprising polyether diol and polyester diol or (b) a polyether polyester diol.
2. The polymer of embodiment 1 wherein the polyether diol comprises at least one type of oxyalkylene sequence selected from the group consisting of oxyethylene, oxypropylene, oxytrimethylene and oxytetramethylene sequences.
3. The polymer of embodiments 1 or 2 wherein the polyether diol is a blend of polyetherdiols.
4. The polymer of embodiments 1 or 2 wherein the polyether diol is not a blend of polyetherdiols.
5. The polymer of any one of embodiments 1-4 wherein the polyether diol is a random copolymer of two or more oxyalkylene sequences.
6. The polymer of any one of embodiments 1-4 wherein the polyether diol is a block copolymer of two or more oxyalkylene sequences, where in optional embodiments the block copolymer is an alternating copolymer of two or more oxyalkylene sequences or is an alternating copolymer of two different oxyalkylene repeat units.
7. The polymer of any one of embodiments 1-6 wherein the diisocyanate is an aliphatic diisocyanate and the reactants do not include an aromatic diisocyanate.
8. The polymer of any one of embodiments 1-6 wherein the diisocyanate is a mixture of aliphatic diisocyanates and the reactants do not include an aromatic diisocyanate.
9. The polymer of any one of embodiments 1-6 wherein the diisocyanate is an aromatic diisocyanate and the reactants do not include an aliphatic diisocyanate.
10. The polymer of any one of embodiments 1-6 wherein the diisocyanate is a mixture of aromatic diisocyanates and the reactants do not include an aliphatic diisocyanate.
11. The polymer of any one of embodiments 1-6 wherein the diisocyanate is a mixture of aromatic diisocyanate and aliphatic diisocyanate.

In one embodiment, the PEU is a polyether ester urethane urea (PEEUU), i.e., a polymer than contains both urethane and urea linkages, and between those urethane and urea linkages are located polyether and polyester groups. The following numbered embodiments provide exemplary PEEUU:
1. A polymer composition which is the reaction product of a diamine and a pre-polymer, where the pre-polymer is the reaction product of a diisocyanate and either (a) a mixture comprising polyether diol and polyester diol or (b) a polyether polyester diol.
2. The polymer of embodiment 1 wherein the polyetherdiol comprises at least one type of oxyalkylene sequence selected from the group consisting of oxyethylene, oxypropylene, oxytrimethylene and oxytetramethylene sequences.
3. The polymer of embodiments 1 or 2 wherein the polyetherdiol is a blend of polyetherdiols.
4. The polymer of embodiments 1 or 2 wherein the polyether diol is not a blend of polyetherdiols.
5. The polymer of any one of embodiments 1-4 wherein the polyetherdiol is a random copolymer of two or more oxyalkylene sequences.
6. The polymer of any one of embodiments 1-4 wherein the polyetherdiol is a block copolymer of two or more oxyalkylene sequences, where in optional embodiments the block copolymer is an alternating copolymer of two or more oxyalkylene sequences or is an alternating copolymer of two different oxyalkylene repeat units.
7. The polymer of any one of embodiments 1-6 wherein the diisocyanate is an aliphatic diisocyanate and the reactants do not include an aromatic diisocyanate.
8. The polymer of any one of embodiments 1-6 wherein the diisocyanate is a mixture of aliphatic diisocyanates and the reactants do not include an aromatic diisocyanate.
9. The polymer of any one of embodiments 1-6 wherein the diisocyanate is an aromatic diisocyanate and the reactants do not include an aliphatic diisocyanate.
10. The polymer of any one of embodiments 1-6 wherein the diisocyanate is a mixture of aromatic diisocyanates and the reactants do not include an aliphatic diisocyanate.
11. The polymer of any one of embodiments 1-6 wherein the diisocyanate is a mixture of aromatic diisocyanate and aliphatic diisocyanate.
12. The polymer of any one of embodiments 1-11 wherein the diamine is an aliphatic diamine.
13. The polymer of any one of embodiments 1-11 wherein the diamine is a polyether diamine.
14. The polymer of any one of embodiments 1-11 wherein the diamine is a blend of diamines.
15. The polymer of any one of embodiments 1-11 wherein the diamine is a blend of aliphatic diamine and polyether diamine.

The following provides additional embodiments of PEU as well as describing component parts of embodiments of PEU:
1. A hydroswellable, segmented, aliphatic polyurethane PEU composition, comprising at least a first and a second different polyoxyalkelene chain segment covalently linked by a second group of chain segments including aliphatic urethane segments, wherein said composition swells at least 5% when immersed in water. Such a PEU may be prepared by reacting a polyoxyalkylene (with hydroxyl end groups) with diisocyanate to create a high molecular weight polymer.
2. A hydroswellable, segmented, aliphatic polyether-urea PEU composition, comprising at least a first and a second different polyoxyalkylene chain segment covalently linked by a second group of chain segments including aliphatic urea segments, wherein said composition swells at least 5% when immersed in water. Such a PEU could be prepared by reacting a polyetheramine with diisocyanate to create urea linkages in the absence of urethane linkages (the latter of which are the byproduct of reacting hydroxyl groups with isocyanate groups).
3. A hydroswellable, segmented, aliphatic polyetherurethane-urea PEU composition, comprising at least a first and a second different polyoxyalkylene chain segment covalently linked to form a multiblock copolymer, which is covalently linked by a second group of chain segments including both aliphatic urethane and urea segments, wherein said composition swells at least 5% when immersed in water.
4. A hydroswellable, segmented, aliphatic polyurethane PEU composition, comprising at least one polyoxyalkylene chain segment and one polycarbonate chain segment covalently linked by a second group of chain segments including both aliphatic urethane and urea segments, wherein said composition swells at least 5% when immersed in water.
5. A hydroswellable, segmented, aliphatic polyurethane-urea PEU composition, comprising at least one polyoxyalkelene chain segment and one polycarbonate chain segment covalently linked by a second group of aliphatic urethane segments, wherein said composition swells at least 5% when immersed in water.
6. A PEU wherein a polyoxyalkylene chain segment comprises segments derived from at least one polyoxyalkylene selected from the group consisting of poly(ethylene glycol), poly(propylene glycol), poly(ethylene glycol)-block-poly(propylene glycol), poly(propylene glycol)-block-poly(ethylene glycol)-block-poly(propylene glycol), and poly(ethylene glycol-ran-propylene glycol), and optionally including poly(ethylene glycol)-block-poly(propylene glycol)-block-poly(ethylene glycol).
7. A PEU wherein polyoxyalkylene chain segment comprises segments derived from at least one polyoxyalkylene selected from the group consisting of poly(tetramethylene glycol-block-ethylene glycol), poly(tetramethylene glycol-block-propylene glycol), poly(ethylene glycol-block-tetramethylene glycol-block-ethylene glycol), poly(trimethylene glycol), poly(pentamethylene glycol), and poly(hexamethylene glycol), and optionally including poly(tetramethylene glycol).
8. A PEU wherein urethane segments are derived from at least one diisocyanate selected from the group consisting of isophorone diisocyanate, 4,4′-methylenediphenyl diisocyanate, m-phenylene diisocyanate, p-phenylene diisocyanate, 2,6-TDI, 2,4-TDI, 1,5-Naphthalene diisocyanate, 4,4-MDI, 2,4-MDI, 2,2,-MDI, MDI, Tolidine diisocyanate, dianisidine diisocyanate, p-xylene diisocyanate, m-xylene diisocyanate, m-TMXDI (1,3-Bis(1-isocyanato-1-methylethyl)benzene), p-TMXDI (1,4-Bis(1-isocyanato-1-methylethyl)benzene), 1,5-diisocyanato-2-methylpentane, lysine diisocyanate methyl ester, 2,2,4-trimethylhexane 1,6-diisocyanate, 2,4,4-trimethylhexane 1,6-diisocyanate, 2,5(6)-Bis(isocyanatomethyl)bicycle[2.2.1]heptane, 1,3,3-trimethyl-1-(isocyanatomethyl)-5-isocyanatocyclohexane, 1,3-diisocyanato-2,4-dimethyloctane, octahydro-4,7-methano-1H-indenedimethyl diisocyanate, 1,1′-methylenebis(4-isocyanatocyclohexane), poly(ethylene oxide)diisocyanate, and poly(propylene oxide)diisocyanate, and wherein the resulting polyoxyalkylene urethane molecules have isocyanate terminal groups that are chain-extended with an alkylene diamine selected from the group consisting of ethylene-, trimethylene-, tetramethylene-, hexamethylene-, and octamethylene-diamine, thereby forming polyetherurethane-urea segmented chains.
9. A PEU which comprises urethane segments derived from at least one diisocyanate selected from the group consisting of hexamethylene diisocyanate, octamethylene diisocyanate, decamethylene diisocyanate, dodecamethylene diisocyanate, 1,4-cyclohexane diisocyanate, and cyclohexane bis(methylene isocyanate), and wherein the resulting polyoxyalkylene urethane molecules have isocyanate terminal groups that are chain-extended with a polyetheramine selected from the group consisting of poly(ethylene glycol)diamine, poly(propylene glycol)diamine, poly(ethylene-co-propylene glycol)diamines, poly(trimethylene glycol)diamine, poly(tetramethylene glycol)diamines, poly(pentamethylene glycol)diamines, and poly(hexamethylene glycol)diamine, thereby forming polyetherurethane-urea segmented chains.
10. A PEU which comprises urethane segments derived from at least one diisocyanate selected from the group consisting of isophorone diisocyanate, 4,4′-methylenediphenyl diisocyanate, m-phenylene diisocyanate, p-phenylene diisocyanate, 2,6-TDI, 2,4-TDI, 1,5-Naphthalene diisocyanate, 4,4-MDI, 2,4-MDI, 2,2,-MDI, MDI, Tolidine diisocyanate, dianisidine diisocyanate, p-xylene diisocyanate, m-xylene diisocyanate, m-TMXDI (1,3-Bis(1-isocyanato-1-methylethyl)benzene), p-TMXDI (1,4-Bis(1-isocyanato-1-methylethyl)benzene), 1,5-diisocyanato-2-methylpentane, lysine diisocyanate methyl ester, 2,2,4-trimethylhexane 1,6-diisocyanate, 2,4,4-trimethylhexane 1,6-diisocyanate, 2,5(6)-Bis(isocyanatomethyl)bicycle[2.2.1]heptane, 1,3,3-trimethyl-1-(isocyanatomethyl)-5-isocyanatocyclohexane, 1,3-diisocyanato-2,4-dimethyloctane, octahydro-4,7-methano-1H-indenedimethyl diisocyanate, 1,1′-methylenebis(4-isocyanatocyclohexane), poly(ethylene oxide)diisocyanate, and poly(propylene oxide)diisocyanate, and wherein the resulting polyoxyalkylene urethane molecules have isocyanate terminal groups that are chain-extended with a polyetheramine selected from the group consisting of poly(ethylene glycol)diamine, poly(propylene glycol)diamine, poly(ethylene-co-propylene glycol)diamines, poly(trimethylene glycol)diamine, poly(tetramethylene glycol)diamines, poly(pentamethylene glycol)diamines, and poly(hexamethylene glycol)diamine, thereby forming polyetherurethane-urea segmented chains.
11. A PEU which comprises polycarbonate chain segments derived from at least one polycarbonate selected from the group consisting of poly(1,6-hexyl-1,2-ethyl-carbonate)diol, poly(1,6-hexyl-carbonate)diol, poly(1,2-ethyl-carbonate)diol, poly(1,4-butyl-carbonate)diol, poly(1,5-pentyl-carbonate)diol, and poly(trimethylene carbonate)diol.

### Polymer and Multilayer Composition Properties

In one embodiment, the present disclosure provides a multilayer polymeric composition wherein multiple layers, i.e., at least two layers and optionally three layers and still further optionally more than three layers, of polymeric material are adjacent to one another. The composition may be described as anisotropic, in that the properties of the composition is directionally dependent in a direction from one side of the composition, through the two or more layers, to the other side of the composition. In other words, the properties of any one layer are different from the properties of an adjacent layer.

Each layer may contain a single polymer or it may contain a blend of different polymers. In the case when the layer is formed from a single polymer, then the properties of the layer will largely reflect the properties of the single polymer. However, when the layer is formed from a blend of polymers, then the layer will have properties unlike either of the components of the blend. The polymer, for example the first polymer and/or the second polymer and/or the third polymer, any of which may optionally be a PEU, may be described in terms of its properties in addition to, or instead of, being described in terms of its chemical composition and/or its method of manufacture. Likewise, each layer in the composition may be described in terms of its properties in addition to, or instead of, being described in terms of its chemical composition and/or its method of manufacture. One or more of the following properties may be used to characterize any of the polymers including PEU and specific PEU embodiments described herein, where in various aspects each property used to characterize a polymer, e.g., a PEU, may have a value or range of values as stated below. Likewise, the properties of a layer, may be described in part by one or more of the following exemplary properties.

The polymer or layer may be described in terms of its hydrophilicity, i.e., the extent to which it absorbs water. As used herein, reference to the hydrophilic nature of a polymer or a layer may be considered to be a reference to the percent water absorption of the polymer or layer. Greater hydrophilicity is associated with greater water absorption, and these terms are often used interchangeably. Accordingly, when the term hydrophilic is used herein, as an alternative, the term percent water absorption can be substituted for the term hydrophilic.

As preferably used herein, the extent to which a polymer or layer absorbs water may be measured on a mass basis, and this measurement is a reflection of the hydrophility of the polymer or layer. A suitable test for measuring water absorption is to prepare film strips that are weighed to determine an initial weight. The strips are submerged in a solution of 1% methyl cellulose dissolved in deionized water for 16 hours at 37° C. The film strips are removed and blotted dry, and the final weight is recorded. The final weight is subtracted from the initial weight, and the difference is divided by the initial weight and then multiplied by 100 to determine the percentage of water that is absorbed by the film strips relative to the initial weight. In various aspects, the water uptake is greater than 50%, or greater than 55%, or greater than 60%, or greater than 65%, or greater than 70%, or greater than 75%, or greater than 80%, or greater than 85%, or greater than 90%.

Another way in which water absorptivity may be measured is by the extent to which a sample swells in water. For instance, the polymer may be hydroswellable, or in other words, when a sample of a specified volume is placed into pure water, the sample will absorb water and swell to a larger volume. In various aspects, the sample of polymer of layer swells to a volume that is at least 10%, or at least 20%, or at least 30%, or at least 40%, or at least 50%, or at least 60%, or at least 70%, or at least 80%, greater than its initial volume. In various aspects, suitable ranges are 40-80% swelling, 50-70% swelling, or 55-65% swelling.

The extent to which a sample of polymer or layer swells may also be measured in terms of a change in thickness of a sample when that sample is exposed to moisture. A suitable test method to measure increase in thickness due to swelling is to prepare film strips and then determine their initial dimensions, specifically the thickness. The strips are submerged in a solution of 1% methyl cellulose dissolved in deionized water for 16 hours at 37° C. The film strips are removed and blotted dry, and the final thickness is measured. The final thickness is subtracted from the initial thickness, and the difference is divided by the initial thickness and then multiplied by 100 to determine the percent increase in thickness of the film strips relative to the initial dimension. In various aspects, the increase in thickness is greater than 5%, or greater than 10%, or greater than 15%.

In one embodiment, the polymer or layer may be characterized in terms of whether, or the extent to which, the polymer absorbs or degrades or is structurally stable in a biological environment. In one embodiment, the polymer is non-absorbable, or in other words, is bio-stable. A bio-stable PEU is particularly useful when implantation of polymer is desired for long-term performance, e.g., as cartilage replacement. As used herein, a polymer or layer is bio-stable if it experiences less than 5% weight loss over a six month period when exposed to biological fluid at 37° C. In general, removing ester linkages from a polymer will increase the bio-stability of the polymer. When some degree of absorbable performance is desired of the polymer, a polyester segment made from, e.g., glycolide or substituted glycolide, may be included in the polymer. In general, a bio-stable polymer as identified herein will be more bio-stable than a polyester formed entirely from L-lactide and glycolide.

In one particular embodiment, the percent water absorption gradually increases outwardly from the deepest layer to the surface layer for the fully hydrated multilayer composition. In one convention used herein, the deepest layer refers to that layer which is adjacent to the bone, e.g., directly adjacent to the femoral head, and is also referred to herein as the first layer. The percent water absorption for the deepest layer is preferably greater than 50%, and preferably between 50 and 100%, more preferably between 50 and 90%, and more preferably between 50 and 80%. The percent water absorption for the middle layer (or second layer) is greater than the percent water absorption for the deepest (or first) layer, and preferably greater than 60%, and preferably between 60 and 90%, more preferably between 65 and 85%, and more preferably between 70 and 85%. The percent water absorption for the surface layer (e.g., the third layer in a trilayer construction) is greater than the percent water absorption for the middle layer. Furthermore, the percent water absorption for the surface layer is preferably greater than 70%, preferably between 70 and 110%, more preferably between 75 and 100%, and more preferably between 80 and 100%.

In general, when it is desired that a layer demonstrate greater lubricity, then greater lubricity can be achieved by increasing the hydrophilicity of that layer. In order to mimic articular cartilage as closely as possible, it is desirable for the layer to have good lubricity, and hence to have good hydrophilicity, i.e., good water absorption. In general, the oxyethylene units impart hydrophilicity, i.e., enhanced percent water absorption, to a polymer, so that increasing the hydrophilicity of a polymer or layer can be achieved by including more oxyethylene units in the polymer or layer, where oxyethylene units are readily introduced into a polymer by including polyethylene glycol as a reactant.

In one embodiment, a polymer that forms a layer may be characterized by its inherent viscosity. For example, the inherent viscosity of a polymer may be measured according to the procedure described in ASTM D2857-95. In various aspects, the inherent viscosity of the polymer is greater than 2 dl/g, or greater than 2.5 dl/g, or greater than 3 dl/g, or greater than 3.5 dl/g, or greater than 4 dl/g, or greater than 4.5 dl/g, or greater than 5 dl/g. In various aspects, the inherent viscosity may be as high as 10 dl/g, or as high as 9 dl/g, or as high as 8 dl/g, or as high as 7 dl/g. Thus, suitable exemplary ranges are 2-10 dl/g, or 3-8 dl/g, or 4-7 dl/g.

In one embodiment, the polymer or layer may be characterized by its coefficient of friction (COF). COF may be measured according to the procedure described in ASTM D1894. In various aspects, the COF of the polymer or layer is less than 0.2, or less than 0.15, or less than 0.1, or less than 0.05, or less than 0.03. In other aspects, the COF of the polymer or layer is within the range of 0.001 to 0.20, or within the range of 0.001 to 0.18, or within the range of 0.001 to 0.15, or within the range of 0.005 to 0.10.

In one embodiment, the polymer or layer may be characterized by its burst properties. For example, the burst strength of the polymer or layer may be measured according to a modified version of ASTM D3787-07, Standard Test Method for Bursting Strength of Textiles-Constant-Rate-of-Traverse (CRT) Ball Burst Test, in which the modified version of this method is conducted using a testing apparatus that is an MTS Synergie equipped with a ball burst test fixture. The fixture consists of an upper ball portion and a lower fixture plate for securing the film sample, wherein the upper ball portion is a plunger of diameter 11.4 mm and the lower fixture plate has a circular hold of diameter 20 mm for accepting said plunger. The ball portion of the test fixture is attached to the MTS Synergie and the system is zeroed to account for the mass of the fixture. The top clamp of the fixture plate is removed and the film sample with a thickness of approximately 0.60 mm is placed on the ball burst fixture base. Next, the sample is centered within the threaded holes used to attach the top clamp plate. The top clamp plate is attached over the film, and the sample is secured in the fixture by tightening the four socket head cap screws using an Allen wrench ( 3/16″). The test is initiated by manually lowering the upper ball portion of the test fixture to contact the film, providing a 0.1 N preload. The plunger is lowered at a rate of 1 inch per minute onto the film sample until the film fails, at which point the ball portion penetrates the opening in the lower fixture plate to complete the test. Under these test conditions, in various aspects, the polymer may have a minimum extension (measured at peak load during burst testing using a wet sample of polymer) of greater than 30 mm, or greater than 35 mm, or greater than 40 mm, or greater than 45 mm, or greater than 50 mm, or greater than 55 mm, or greater than 60 mm, or greater than 65 mm, or greater than 70 mm. This same test method may be used to measure the peak load of a wet polymer sample, where in various aspects the peak load is greater than 70 N, or greater than 75 N, or greater than 80 N, or greater than 85 N, or greater than 90 N, or greater than 95 N, or greater than 100 N, or greater than 105 N, or greater than 110 N, or greater than 115N, or greater than 120 N, or greater than 125 N, or greater than 130 N.

In one embodiment, the polymer or layer may be analyzed by differential scanning calorimetry (DSC) and/or associated thermal transitions. To make such analysis, samples weighing approximately 5-10 milligrams are loaded into a differential scanning calorimeter and heated at a controlled rate (e.g. 10° C./min, 15° C./min, or 20° C./min) from 0° C. to 230° C. The sample can be quenched by immediate cooling in liquid nitrogen, or can be cooled at a controlled rate (e.g. 10° C./min, 15° C./min, or 20° C./min) to a reduced temperature at or below room temperature. Upon cooling, the sample can be reheated at a controlled rate (10° C./min, 15° C./min, or 20° C./min) to 230° C. in order to obtain thermal data reflecting the absence of a thermal history. This type of method provides data related to both the thermal history of the sample (first run) and data that also reflects the absence of a thermal history (second run). Under these test conditions, in various aspects, the polymer may have an endothermic phase transition (melting event) below 100° C., or below 80° C., or below 70° C., or below 65° C., or below 60° C., or below 55° C. This same test method may be used to measure the heat of melting of a PEU soft segment, where in various aspects the heat of melting is between 1 joules/gram and 50 joules/gram, or between 5 joules/gram and 40 joules/gram, or between 5 joules/gram and 30 joules/gram, or between 5 joules per gram and 25 joules/gram, or between 10 joules per gram and 25 joules/gram.

Compressive modulus for all layers should preferably fall within the range of 0.1 MPa to 30 MPa, and the compressive modulus should preferably gradually increase outwardly from the deep (innermost) layer to the surface layer. Ranges for compressive modulus for all layers should preferably be between 0.1 MPa and 30 MPa, or 0.1 MPa and 20 MPa, or 0.1 MPa and 15 MPa, or 0.5 MPa and 20 MPa or 1 MPa and 20 MPa, or 2 MPa and 15 MPa; preferably greater than 0.1 MPa and no greater than 25 MPa. In one embodiment there is at least a difference in compressive modulus of 0.5 MPa for each layer. For instance, the deep layer would have a compressive modulus at least 0.5 MPa greater than the middle layer, and the middle layer would have a compressive modulus at least 0.5 MPa greater than the surface layer. More preferably there should be a difference in compressive modulus of 1.0 MPa between each layer, wherein the deep layer demonstrates the highest compressive modulus.

In one embodiment, the compressive modulus of the layers gradually decrease from the innermost deep layer to the outermost surface layer, while optionally and additionally the water absorption gradually increases from the deep layer to the surface layer, and optionally and additionally the Shore hardness gradually increases from the innermost deep layer to the outermost surface layer. Accordingly, the multilayer composition may anisotropic in terms of compressive modulus and/or water absorption and/or hardness.

Optionally, in any of the compositions and caps described herein, the innermost layer (i.e., the layer intended to be directly adjacent to the head of the femoral bone or equivalent) of the multilayer composition can partially bioabsorbable, such as, for example, wherein the inner layer is a blend of (1) a polyether ester urethane urea (PEEUU) and (2) a biostable polyether urethane urea, where the polyether ester urethane urea degrades slowly over time to create a porous microstructure allowing for cellular ingrowth to provide improved fixation of the femoral cap to the femoral head; in further optional embodiments, growth factors, peptides, and/or pharmaceutical agents, for example, that promote osteogenesis and/or cartilage growth are incorporated within the innermost layer to facilitate the ingrowth of chrondrocytes and/or osteocytes. Suitable pore sizes range from 100-500 micrometers in diameter to allow for cellular ingrowth.

In one embodiment, the composition has three layers, and the middle layer has the lowest compressive modulus and Shore hardness, but the highest water absorption relative to the innermost and outermost layers.

In one embodiment, the composition has three layers, and the middle layer has the highest compressive modulus and Shore hardness, but the lowest water absorption relative to the innermost and outermost layers.

Optionally, the multilayer composition may be anisotropic in terms of compressive modulus and/or water absorption and/or hardness. The compressive modulus of the layers gradually decrease from the innermost deep layer to the outermost surface layer, while optionally and additionally the water absorption gradually increases from the deep layer to the surface layer, and optionally and additionally the Shore hardness gradually decreases from the innermost deep layer to the outermost surface layer. For example, the compressive modulus may decrease from the innermost deep layer to the outermost surface layer, and the compressive modulus ranges from 1-10 MPa in the innermost deep layer, 1-5 MPa in the middle layer, and 0.1-1 MPa in the outermost surface layer; while tensile modulus among the layers may vary, where the tensile modulus of the innermost layer ranges from 1-5 MPa, middle layer ranges from 5-25 MPa, and outermost surface layer ranges from 10-50 MPa, i.e., the tensile modulus increases from innermost layer to outermost layer. These values may be tailored to mimic natural cartilage.

The multilayer composition will have a thickness, sometimes a varying thickness as viewed from the centerpoint of the composition to the edge of the composition, or from the apex of the cap to the edge of the rim of the cap. That thickness should, in one embodiment, conform to the width of the joint where the multilayer composition is to be placed. That thickness varies considerably among patients, and depends on the particular joint and its condition. See, for example, Lesquesne et al. Ann. Rheum. Dis. (2004) vol. 63, pages 1145-1151. The joint space width will maximize the thickness of the multilayer composition. Compositions of different thicknesses may be prepared for different indications. For example, if the patient has a bone on bone joint, then such a patient could tolerate a thicker cap, whereas the same patient with little damage would need a thinner cap. In one embodiment, the multilayer composition has a thickness of 0.5 mm to 4.0 mm; while in another embodiment the thickness is 1.0 to 3.0 mm; while in another embodiment the composition has a thickness of 1.5 to 2.0 mm, where thickness is measured after the multilayer composition has absorbed water and reached an equilibrium content of water. In one embodiment where the composition is in the form of a cap, the thickness of the composition is constant in a direction from the apex of the cap to the edge of the rim of the cap.

Each layer of the multilayer composition may be characterized by its hardness. Hardness may be measured with a durometer according to the procedure described in ASTM D2240, with the result called a Shore hardness on either the A or D scale. Shore hardness for all layers should preferably fall within the range of 20 Shore A to 95 Shore D, and the Shore hardness should preferably gradually increase outwardly from the deep (innermost) layer to the surface layer. In various embodiments, ranges for Shore hardness for all layers are between 20 Shore A (20A) to 79 Shore D (79D), or 25A and 79D, or 30A and 79D, or 40A and 79D or 50A and 79D, or 60A and 79D, or 70A and 79D, or 80A and 79D, or 90A and 79D, or 95A and 79D, or 30A and 75D, or 40A and 65D, or 40A and 55D, or 40A and 45D, or 40A and 90A, or 40A and 80A, or 50A and 67D, or 50A and 55D, or 50A and 95A, or 50A and 90A, or 50A and 80A, or 50A and 75A, or 50A and 70A; preferably greater than 30 Shore A and preferably no greater than 79 Shore D. In one embodiment, no layer is harder than Shore D 70.

In one embodiment there is at least a difference in Shore hardness of 2 Shore A units for each layer. For instance, the deep layer has a Shore A hardness at least 2 Shore A units less than the middle layer, and the middle layer would have a Shore A hardness of at least 2 shore A units less than the surface layer. In various embodiments, there is at least a difference in Shore A hardness of 5 Shore A units between each layer, wherein the deep layer demonstrates the lowest Shore hardness. In various embodiments, ranges for the Shore hardness of the innermost layer may be between 30A and 79D, or 30A and 65D, or 30A and 95A, or 30A and 85A or 30A and 80A. Ranges for the Shore hardness of the middle layer should preferably be between 35A and 79D, or 35A and 65D, or 35A and 55D, or 35A and 95A, or 35A and 80A. Ranges for the Shore hardness of the outermost layer (which may be the second, third, etc. layer) may be between 35A and 79D, or 35A and 65D, or 35A and 55D, or 35A and 95A, or 35A and 80A.

Optionally, the multilayer composition may be marked so that it appears to have a directionality or orientation. In one embodiment, the multilayer composition comprises arrow-like indicia. For example, the arrows may point toward the front of the subject when the composition is properly placed within a patient. In another embodiment, the multilayer composition comprises a series of parallel lines where the lines may be, e.g., solid or dashed or dotted. The separation of the parallel lines may increase with increased stretching of the multilayer composition, where the degree of increased spacing between the parallel lines may be used as a visual indicator of the degree to which the multilayer composition has been stretched. The directionality of the parallel lines may also serve as an aid to the surgeon who is placing the composition within a patient, in order to aid the surgeon in placing the composition properly in the patient. Optionally, the markings may be formed by colorant, e.g., black, blue, green, and optionally multiple colors may be used. Alternatively, or in addition, the markings may be formed by texture, e.g., the arrow or lines may appear as a raised or indented surface relative to the surface that surrounds the arrow or lines. In another embodiment, the marking(s) are made separately from the multilayer composition and are subsequently bonded onto the composition, e.g., the marking(s) may be placed within the composition or on a surface of the composition. In another embodiment, the multilayer composition may be tinted, which includes partially tinted. For example, the multilayer composition may be tinted red on the side of the composition which desirably faces left in a patient, and blue on the side of the composition which desirably faces right in a patient. As another alternative, the bottom of the composition, i.e., the side of the composition which sits directly on bone, may be colored or otherwise marked differently from the top of the composition, in order to assist the surgeon in proper placement of the composition within the patient, e.g., within the joint of a patient. Regardless of whether the composition has marking(s), the composition may or may not rotate freely after it is implanted in the patient. Thus, the marking may aid in the proper initially placement of the multilayer composition, even though the markings may change location over time, as the implant moves.

In order to increase or decrease the performance property of a polymer or a layer that contains the polymer, the composition of the polymer should be modified. The Table below illustrates how changes in composition will affect various properties, and provides guidance as to approximately what ranges of composition will provide what ranges of properties.

TABLE

| | | | Swelling | | | | Burst |
|---|---|---|---|---|---|---|---|
| | | | (% water | | | Burst Test: | Test: Max |
| Polymer | Percent | I.V.² | uptake, by | | Percent | Peak Load⁵ | Extension⁶ |
| Class | Polyether¹ | (dL/g) | mass)³ | COF | Polycarbonate⁴ | (N) | (mm) |
| Polyether | 50-99% | >2.0 dl/g | >30% | <.15 | NA | >80 N | >40 mm |
| Urethane | 65-99% | >3.5 dl/g | >40% | <.10 | | >90 N | >50 mm |
| (PEUT) | 75-99% | >5.0 dl/g | >50% | <.05 | | >120 N | >60 mm |
| | 85-99% | | | | | | |
| Polyether | 50-99% | >2.0 dl/g | >30% | <.15 | NA | >80 N | >40 mm |
| Urea | 65-99% | >3.5 dl/g | >40% | <.10 | | >90 N | >50 mm |
| (PEUA) | 75-99% | >5.0 dl/g | >50% | <.05 | | >120 N | >60 mm |
| | 85-99% | | | | | | |
| Polyether | 50-99% | >2.0 dl/g | >30% | <.15 | NA | >80 N | >40 mm |
| Urethane | 65-99% | >3.5 dl/g | >40% | <.10 | | >90 N | >50 mm |
| Urea | 75-99% | >5.0 dl/g | >50% | <.05 | | >120 N | >60 mm |
| (PEUU) | 85-99% | | | | | | |
| Polyether | 50-99% | >2.0 dl/g | >30% | <.15 | 50-99% | >80 N | >40 mm |
| Carbonate | 65-99% | >3.5 dl/g | >40% | <.10 | 55-90% | >90 N | >50 mm |
| Urethane | 75-99% | >5.0 dl/g | >50% | <.05 | 60-85% | >120 N | >60 mm |
| Urea | 85-99% | | | | 65-75% | | |
| (PECUU) | | | | | | | |

¹Per repeat unit, by mass
²Inherent viscosity
³% water uptake, by mass
⁴% of total soft segments, by mass
⁵Peak load determined from burst testing; performed on wet test specimen
⁶Maximum extension measured at peak load during burst testing; performed on wet test specimen

### Method of Making PEU Polymers

The PEU polymers may be prepared by reacting a diisocyanate with one or both of a diol and a diamine. The diol may be, for example, a polyether diol, i.e., a polyether segment flanked by two hydroxyl groups, to thereby provide for incorporation of polyether functionality into the PEU. The diol may be a polyether polyester diol, i.e., a polyether segment flanked on either end by a polyester segment where each of the two polyester segments terminate with a hydroxyl group, to thereby provide for incorporation of both polyether and polyester functionality into the PEU. The diol may be a polyether carbonate diol, i.e., a polyether segment that is joined to at least two carbonate groups, the polyether carbonate diol having two terminal hydroxyl groups to thereby provide for incorporation of both polyether and polycarbonate functionality into the PEU.

Representative examples of synthesis techniques that may be adapted to prepare PEUs are provided in US 2010/0056646 and US 2009/0233887, both of which are incorporated by reference in their entirety.

The PEU may be sterilized prior to, or preferably after, being packaged for shipment. For example, the PEU may be exposed to radiation such as gamma rays or E-beams for a sufficient period of time to achieve sterilization. Alternatively, or additionally, the PEU may be sterilized by exposing the PEU to chemical sterilization agents, e.g., ethylene oxide.

### Bioactive Agents

The polymers and/or multilayer compositions provided herein may be in combination with one or more bioactive agents. The bioactive agents may be incorporated into or onto the polymer or layer by a variety of methods, including for example, by applying the bioactive agent to the polymer or layer (e.g., coating, painting, dipping or spraying the polymer onto one or more surfaces or a portion of a surface of the polymer), and/or by incorporating the bioactive agent, or a composition comprising the bioactive agent within the polymer or layer (e.g, by admixing the bioactive agent within the polymer, or a portion of the polymer during formation of the polymer, or by admixing the bioactive agent with one or more polymers and incorporating these polymers into the final polymer). Since the polymers are preferably hydroswellable, bioactive agent may be incorporated into the polymer at the same time that polymer absorbs water. For example, the bioactive agent may be dissolved in a saline/water solution, or may be formed into an aqueous dispersion of liposome or micelle in the case of a hydrophobic bioactive agent. The polymer may then be placed into the bioactive agent solution/dispersion, and the bioactive agent will enter the polymer along with the water. Within certain embodiments the bioactive agent is designed to be released from the polymer or layer over a desired time frame.

Examples of such bioactive agents includes, but are not limited to, fibrosis-inducing agents, antifungal agents, antibacterial agents and antibiotics, anti-inflammatory agents, anti-scarring agents, immunosuppressive agents, immunostimulatory agents, antiseptics, anesthetics, antioxidants, cell/tissue growth promoting factors, anti-neoplastic, anticancer agents and agents that support ECM integration.

Examples of fibrosis-inducing agents include, but are not limited to talcum powder, metallic beryllium and oxides thereof, copper, silk, silica, crystalline silicates, talc, quartz dust, and ethanol; a component of extracellular matrix selected from fibronectin, collagen, fibrin, or fibrinogen; a polymer selected from the group consisting of polylysine, poly(ethylene-co-vinylacetate), chitosan, N-carboxybutylchitosan, and RGD proteins; vinyl chloride or a polymer of vinyl chloride; an adhesive selected from the group consisting of cyanoacrylates and crosslinked poly(ethylene glycol)-methylated collagen; an inflammatory cytokine (e.g., TGF, PDGF, VEGF, bFGF, TNFa, NGF, GM-CSF, IGF-a, IL-1, IL-1-, IL-8, IL-6, and growth hormone); connective tissue growth factor (CTGF); a bone morphogenic protein (BMP) (e.g., BMP-2, BMP-3, BMP-4, BMP-5, BMP-6, or BMP-7); leptin, and bleomycin or an analogue or derivative thereof.

Optionally, the polymer or layer may additionally comprise a proliferative agent that stimulates cellular proliferation. Examples of proliferative agents include: dexamethasone, isotretinoin (13-cis retinoic acid), 17-estradiol, estradiol, 1-a-25 dihydroxyvitamin D3, diethylstibesterol, cyclosporine A, L-NAME, all-trans retinoic acid (ATRA), and analogues and derivatives thereof. (see US 2006/0240063, which is incorporated by reference in its entirety).

Examples of antifungal agents include, but are not limited to, polyene antifungals, azole antifungal drugs, and Echinocandins.

Examples of antibacterial agents and antibiotics include, but are not limited to, erythromycin, penicillins, cephalosporins, doxycycline, gentamicin, vancomycin, tobramycin, clindamycin, and mitomycin.

Examples of anti-inflammatory agents include, but are not limited to, non-steriodal anti-inflammatory drugs such as ketorolac, naproxen, diclofenac sodium and fluribiprofen.

Examples of anti-scarring agents include, but are not limited to cell-cycle inhibitors such as a taxane, immunomodulatory agents such as serolimus or biolimus (see, e.g., paras. 64 to 363, as well as all of US 2005/0149158, which is incorporated by reference in its entirety).

Examples of immunosuppressive agents include, but are not limited to, glucocorticoids, alkylating agents, antimetabolites, and drugs acting on immunophilins such as ciclosporin and tacrolimus.

Examples of immunostimulatory agents include, but are not limited to, interleukins, interferon, cytokines, toll-like receptor (TLR) agonists, cytokine receptor agonist, CD40 agonist, Fe receptor agonist, CpG-containing immunostimulatory nucleic acid, complement receptor agonist, or an adjuvant.

Examples of antiseptics include, but are not limited to, chlorhexidine and tibezonium iodide.

Examples of anesthetic include, but are not limited to, lidocaine, mepivacaine, pyrrocaine, bupivacaine, prilocaine, and etidocaine.

Examples of antioxidants include, but are not limited to, antioxidant vitamins, carotenoids, and flavonoids.

Examples of cell growth promoting factors include, but are not limited to, epidermal growth factors, human platelet derived TGF-, endothelial cell growth factors, thymocyte-activating factors, platelet derived growth factors, fibroblast growth factor, fibronectin or laminin.

Examples of antineoplastic/anti-cancer agents include, but are not limited to, paclitaxel, carboplatin, miconazole, leflunamide, and ciprofloxacin.

Examples of agents that support ECM integration include, but are not limited to, gentamicin.

It is recognized that in certain forms of therapy, combinations of agents/drugs in the same polymer or layer can be useful in order to obtain an optimal effect. Thus, for example, an antibacterial and an anti-inflammatory agent may be combined into polymer or layer in order to provide combined effectiveness. Particularly preferred combinations for use within the present invention include a combination of anti-inflammatory and anesthetics, or a combination of anti-inflammatory, anesthetic, and anti-bacterial agents. In some embodiments, one or more bioactive agents (e.g., a fibrosis-inducing drug) are applied to only a specific section or area of the polymer or layer, as opposed to the entire polymer or layer. In other embodiments, two or more drugs are applied to two or more areas of the polymer or layer.

### Method of Applying a Multilayer Composition to a Substrate

Within certain embodiments of the invention, methods are provided for applying the multilayer composition to a desired substrate, also referred to herein as support. Representative examples of suitable substrates include, for example, medical devices, as well as biological surfaces (such as the femoral head). The biological surface may be natural or artificial, for example, the biological surface may be an inner surface of a joint, where the joint is made from the natural bone of the patient, or may be an artificial joint made from metal or ceramic.

The multilayer compositions described herein may be applied to a wide variety of medical devices. Particularly preferred medical devices include artificial joints, including for example, hip joints, knee joints, and the temporomandibular joint. Within certain embodiments of the invention, the multilayer composition is formed as a film, sheet, or cap to fit over the surface of bony structures (e.g., femoral head of the femoral joint), particularly in joints where articular cartilage has degenerated. Preferably, the multilayer composition is formed to help protect damaged, injured, surgically traumatized, or, degenerating cartilage, (see, e.g., US 2010/0125341 and US 2010/059495, which are incorporated by reference in their entirety). Within alternative embodiments of the invention, the multilayer composition may be formed or placed on an artificial joint, in order to extend and/or otherwise enhance the effective life of the joint. Representative examples of artificial joints are described in U.S. Pat. Nos. RE 28,895, 7,963,998 and 7,771,485. Within particularly preferred embodiments of the invention, multilayer compositions which are placed over the surface of a subject's cartilage (e.g., joint, femoral head of the femoral joint, etc.) or on a medical device, will have a similar coefficient of friction to that of a normal joint, and will help to at least partially restore both normal joint function and eliminate or reduce pain associated with the joint. Particularly preferred first, second, third etc. polymers are PEU polymers, for example, PEUT, PEUA, PEUU, PECUT, PECUA, PECUU, PEEUT, PEEUA, and PEEUU.

Compositions and Methods for Application within a Polymer-Containing Joint

As described above, the multilayer compositions described herein may be applied to the existing joint of a patient (e.g., to the femoral head), in order to preserve cartilage, or to an artificially-created joint. The multilayer compositions are designed to be swellable in an aqueous or biological environment, and hence, in certain embodiments of the invention auxiliary compositions are provided for injection into a joint containing a multilayer composition. Representative examples of suitable auxiliary compositions include those containing hyaluronic acid or salts thereof, e.g., sodium hyaluronate, saline, buffered forms of saline, as well as various combinations of these. In addition, within further embodiments the auxiliary composition may further comprise one or more biologically active agents as noted above.

The present invention will be illustrated below with reference to Examples, but is not to be construed as being limited thereto. The Examples illustrate methods to prepare polymers, and illustrate methods to prepare multilayer compositions which may optionally be prepared with the exemplified polymers.

### EXAMPLES

### Example 1

### Synthesis of a Polyether-Urethane

For an initial charge, poly(tetramethylene)glycol (average Mn=2,900, 168.0 grams, 0.057931 moles) and poly(ethylene glycol-block-propylene glycol-block-ethylene glycol) (average Mn=14,600, 72.0 grams, 0.0049315 moles) are added to a 2000 mL resin reaction kettle that is fitted with a three-neck glass lid equipped with a stainless steel stirrer. The contents are heated to 100° C. at a reduced pressure of less than 0.5 mm Hg to remove moisture. Upon drying, the system is purged with nitrogen gas and cooled to room temperature. Approximately 560 mL of N,N-dimethylacetamide are added to the reaction kettle through a glass funnel to dissolve the dried reaction components. The contents are stirred gently for at least 30 minutes in order to create a homogeneous solution and then hexamethylene diisocyanate (15.86 grams, 0.0942938 moles) is added. After stirring for 15 minutes, tin(II) 2-ethyl hexanoate (0.2 M in dioxane, 5.9243 mL, 0.0011849 moles) is added and the resulting mixture is stirred for an additional 15 minutes. The contents are then heated to 100° C. The reaction conditions are maintained for 1.25 hours or until the vessel contents are too viscous to continue stirring. Upon obtaining suitable molecular weight, stirring is stopped and the temperature is decreased from 100° C. to room temperature. The final polymer is extracted with deionized water for 24 hours followed by acetone for at least 1 hour to deactivate unreacted isocyanate end groups and to remove any unreacted monomer. The purified polymer is isolated and dried to constant weight at 55° C. in a vacuum oven.

### Example 2

### Synthesis of a Polyether-Urea from Polyetherdiamine and Diisocyanate

For an initial charge, poly(tetramethylene ether glycol) diamine (average Mn=1400, 0.0579 moles, 81.06 grams) and poly(propylene glycol-block-ethylene glycol-block-propylene glycol) diamine (average Mn=2000, 0.00493 moles, 9.86 grams) are added to a 2000 mL resin reaction kettle that is fitted with a three-neck glass lid equipped with a stainless steel stirrer. The contents are heated to 100° C. at a reduced pressure of less than 0.5 mm Hg to remove moisture. Upon drying, the system is purged with nitrogen gas and cooled to room temperature. Approximately 560 mL of N,N-dimethylacetamide are added to the reaction kettle through a glass funnel to dissolve the dried reaction components. The contents are stirred gently for at least 30 minutes in order to create a homogeneous solution. Hexamethylene diisocyanate (15.86 grams, 0.0942938 moles) is added to the reaction kettle at room temperature and stirred for 15 minutes. Then tin(II) 2-ethyl hexanoate (0.2 M in dioxane, 5.9243 mL, 0.0011849 moles) is added to the solution at room temperature and stirred vigorously until the vessel contents are too viscous to continue stirring. The reaction is kept at room temperature overnight, and on the following day polymer is extracted with deionized water for 24 hours followed by acetone for at least 1 hour to deactivate unreacted isocyanate end groups and to remove the remaining N,N-dimethylacetamide and unreacted monomer. The purified polymer is isolated and dried to constant weight at 55° C. in a vacuum oven.

### Example 3

### Synthesis of a Polyether-Urethane-Urea Prepared by Chain Extending with Polyetheramine (2,2′-(Ethylenedioxy)Bis(Ethylamine))

For an initial charge, poly(tetramethylene)glycol (average Mn=2,900, 168.0 grams, 0.057931 moles) and poly(ethylene glycol-block-propylene glycol-block-ethylene glycol) (average Mn=14,600, 72.0 grams, 0.0049315 moles) are added to a 2000 mL resin reaction kettle that is fitted with a three-neck glass lid equipped with a stainless steel stirrer. The contents are heated to 100° C. at a reduced pressure of less than 0.5 mm Hg to remove moisture. Upon drying, the system is purged with nitrogen gas and cooled to room temperature. Approximately 560 mL of N,N-dimethylacetamide are added to the reaction kettle through a glass funnel to dissolve the dried reaction components. The contents are stirred gently for at least 30 minutes in order to create a homogeneous solution, and then hexamethylene diisocyanate (15.86 grams, 0.0942938 moles) is added. After stirring for 15 minutes, tin(II) 2-ethyl hexanoate (0.2 M in dioxane, 5.9243 mL, 0.0011849 moles) is added and the resulting mixture is stirred for 15 additional minutes, all at room temperature. The contents are then heated to 100° C. and these reaction conditions are maintained for about 1.25 hours. Upon obtaining suitable conversion, the temperature is decreased to room temperature. At room temperature, the prepolymer is chain extended by the addition of 2,2′-(ethylenedioxy)bis(ethylamine) (MW=148.20, 0.031431 moles, 4.6581 grams) while stirring vigorously. The contents are stirred at room temperature until the reaction contents are too viscous to continue stirring. The reaction is allowed to stand overnight at room temperature. The final polymer is extracted with deionized water for 24 hours followed by acetone for at least 1 hour to remove any unreacted monomer and to deactivate unreacted isocyanate end groups. The purified polymer is isolated and dried to constant weight at 55° C. in a vacuum oven.

### Example 4

### Synthesis of a Polyether-Urethane-Urea Prepared from a Polyether Diamine Copolymer Based on Polytetramethylene Ether Glycol and Polypropylene Glycol, and Chain Extended with a Polyethylene Glycol

For an initial charge, poly(tetramethylene ether-block-propylene ether) diamine (average Mn=1400, 0.0628625 moles, 88.01 grams) is added to a 2000 mL resin reaction kettle that is fitted with a three-neck glass lid equipped with a stainless steel stirrer. The contents are heated to 100° C. at a reduced pressure of less than 0.5 mm Hg to remove moisture. Upon drying, the system is purged with nitrogen gas and cooled to room temperature. Approximately 560 mL of N,N-dimethylacetamide are added to the reaction kettle through a glass funnel to dissolve the dried reaction components. The contents are stirred gently for at least 30 minutes in order to create a homogeneous solution, and then hexamethylene diisocyanate (15.86 grams, 0.0942938 moles) is added followed by 15 more minutes of stirring. Then tin(II) 2-ethyl hexanoate (0.2 M in dioxane, 5.9243 mL, 0.0011849 moles) is added and the resulting mixture is stirred for 15 additional minutes, all at room temperature. The contents are then heated to 100° C. and these reaction conditions are maintained for 1.25 hours or until suitable conversion has been attained, and then the temperature is decreased to 25° C. At 25° C. polyethylene glycol (average Mn=1000, 0.031431 moles, 31.43 grams) is added while stirring and allowed to mix for 30 minutes. The temperature is then increased to 80° C. and reacted until the desired molecular weight is obtained at which point the temperature is lowered to room temperature. The reaction is allowed to stand overnight at room temperature, and then the polymer is extracted the following day with deionized water for 24 hours followed by acetone for at least 1 hour to remove any unreacted monomer and to deactivate unreacted isocyanate end groups. The purified polymer is isolated and dried to constant weight at 55° C. in a vacuum oven.

### Example 5

### Synthesis of a Polyether-Urea Prepared from a Polyether Diamine Copolymer Based on Polytetramethylene Ether Glycol and Polypropylene Glycol, and Chain Extended with Ethylene Diamine

For an initial charge, poly(tetramethylene ether-block-propylene ether) diamine (average Mn=1400, 0.057931 moles, 81.10 grams) is added to a 2000 mL resin reaction kettle that is fitted with a three-neck glass lid equipped with a stainless steel stirrer. The contents are heated to 100° C. at a reduced pressure of less than 0.5 mm Hg to remove moisture. Upon drying, the system is purged with nitrogen gas and cooled to room temperature. Approximately 500 mL of anhydrous N,N-dimethylacetamide are added to the reaction kettle through a glass funnel to dissolve the dried reaction components. 2,2′-oxybis-ethanamine (Mn=104, 0.33420 moles, 34.75 grams) and tin(II) 2-ethyl hexanoate (0.2 M in dioxane, 5.9243 mL, 0.0011849 moles) are added to the reaction flask and the contents are stirred gently for at least 30 minutes in order to create a homogeneous solution. Hexamethylene diisocyanate (0.588197 moles, 98.935 grams) is added to the solution dropwise using an addition funnel at room temperature while stirring over a one hour period. The reaction conditions are maintained for 1.25 hours or until suitable conversion has been attained. At 25° C. the prepolymer is chain extended by the addition of ethylene diamine (Mn=60.1, 0.196066 moles, 11.7835 grams) while stirring vigorously. The polymer solution is stirred until it becomes too viscous to continue stirring. The reaction is allowed to stand overnight at room temperature, and then the polymer is extracted the following day with deionized water for 24 hours followed by acetone for at least 1 hour to remove any unreacted monomer and to deactivate unreacted isocyanate end groups. The purified polymer is isolated and dried to constant weight at 55° C. in a vacuum oven.

### Example 6

### Synthesis of a Polyether-Carbonate-Urethane-Urea that is Chain Extended with an Aliphatic Diamine

For an initial charge, poly(hexamethylene-carbonate)diol (average Mn=1000, 57.931 grams, 0.057931 moles) and poly(ethylene glycol-block-propylene glycol-block-ethylene glycol) (average Mn=14,600, 72.0 grams, 0.0049315 moles) are added to a 2000 mL resin reaction kettle that is fitted with a three-neck glass lid equipped with a stainless steel stirrer. The contents are heated to 100° C. at a reduced pressure of less than 0.5 mm Hg to remove moisture. Upon drying, the system is purged with nitrogen gas and cooled to room temperature. Approximately 560 mL of N,N-dimethylacetamide are added to the reaction kettle through a glass funnel to dissolve the dried reaction components. The contents are stirred gently for at least 30 minutes in order to create a homogeneous solution, followed by addition of hexamethylene diisocyanate (15.86 grams, 0.0942938 moles) and further stirring for 15 minutes. Tin(II) 2-ethyl hexanoate (0.2 M in dioxane, 5.9243 mL, 0.0011849 moles) is added to the solution at room temperature and stirred for 15 minutes. The contents are then heated to 100° C. and these reaction conditions are maintained for 1.25 hours or until the vessel contents are too thick to continue stirring. Upon obtaining suitable molecular weight, stirring is stopped and the temperature is decreased from 100° C. to room temperature. At 25° C. the prepolymer is chain extended by the addition of ethylene diamine (Mn=60.1, 0.031431 moles, 1.8890 grams) while stirring vigorously. The polymer solution is stirred until it becomes too viscous to continue stirring. The reaction is allowed to stand overnight at room temperature, and then the polymer is extracted the following day with deionized water for 24 hours followed by acetone for at least 1 hour to remove any unreacted monomer and to deactivate unreacted isocyanate end groups. The purified polymer is isolated and dried to constant weight at 55° C. in a vacuum oven.

### Example 7

### Synthesis of a Polyether-Carbonate-Urethane-Urea that is Chain Extended with a Polyetherdiamine(2,2′-(Ethylenedioxy)Bis(Ethylamine))

For an initial charge, poly(hexamethylene-carbonate)diol (average Mn=1000, 57.931 grams, 0.057931 moles) and poly(ethylene glycol-block-propylene glycol-block-ethylene glycol) (average Mn=14,600, 72.0 grams, 0.0049315 moles) are added to a 2000 mL resin reaction kettle that is fitted with a three-neck glass lid equipped with a stainless steel stirrer. The contents are heated to 100° C. at a reduced pressure of less than 0.5 mm Hg to remove moisture. Upon drying, the system is purged with nitrogen gas and cooled to room temperature. Approximately 560 mL of N,N-dimethylacetamide are added to the reaction kettle through a glass funnel to dissolve the dried reaction components. The contents are stirred gently for at least 30 minutes in order to create a homogeneous solution, followed by addition of hexamethylene diisocyanate (15.86 grams, 0.0942938 moles). After stirring for 15 minutes, tin(II) 2-ethyl hexanoate (0.2 M in dioxane, 5.9243 mL, 0.0011849 moles) is added, the resulting mixture is stirred for an additional 15 minutes, all at room temperature. The contents are then heated to 100° C. and these reaction conditions are maintained for 1.25 hours or until the vessel contents are too thick to continue stirring. Upon obtaining suitable molecular weight, stirring is stopped and the temperature is decreased from 100° C. to room temperature. At 25° C. the prepolymer is chain extended by the addition of 2,2′-(ethylenedioxy)bis(ethylamine) (MW=148.20, 0.031431 moles, 4.6581 grams) while stirring vigorously. The polymer solution is stirred until it becomes too viscous to continue stirring. The reaction is allowed to stand overnight at room temperature, and then the polymer is extracted the following day with deionized water for 24 hours followed by acetone for at least 1 hour to remove any unreacted monomer and to deactivate unreacted isocyanate end groups. The purified polymer is isolated and dried to constant weight at 55° C. in a vacuum oven.

### Example 8

### Synthesis of a Polyether-Urethane-Urea Prepared from a Random Copolymer of Polyethylene Glycol and Polypropylene Glycol and Chain Extended with Hexamethylene Diisocyanate and Ethylene Diamine

For an initial charge, poly(tetramethylene ether)glycol (average Mn=2,900, 168.0 grams, 0.057931 moles) and poly(ethylene glycol-ran-propylene glycol) (average Mn=12000, 59.178 grams, 0.0049315 moles) are added to a 2000 mL resin reaction kettle that is fitted with a three-neck glass lid equipped with a stainless steel stirrer. The contents are heated to 100° C. at a reduced pressure of less than 0.5 mm Hg to remove moisture. Upon drying, the system is purged with nitrogen gas and cooled to room temperature. Approximately 560 mL of N,N-dimethylacetamide are added to the reaction kettle through a glass funnel to dissolve the dried reaction components. The contents are stirred gently for at least 30 minutes in order to create a homogeneous solution. Hexamethylene diisocyanate (15.86 grams, 0.0942938 moles) is added followed by 15 minutes of stirring and then tin(II) 2-ethyl hexanoate (0.2 M in dioxane, 5.9243 mL, 0.0011849 moles) is added followed by an additional 15 minutes of stirring, all at room temperature. The contents are then heated to 100° C. and maintained at these reaction conditions for 1.25 hours or until suitable conversion has been attained, and then the temperature is decreased to 25° C. At 25° C. the prepolymer is chain extended by the addition of ethylene diamine (Mn=60.1, 0.031431 moles, 1.8890 grams) while stirring vigorously. The polymer solution is stirred until it has become too viscous to continue stirring. The reaction is allowed to stand overnight at room temperature, and then the polymer is extracted the following day with deionized water for 24 hours followed by acetone for at least 1 hour to remove any unreacted monomer and to deactivate unreacted isocyanate end groups. The purified polymer is isolated and dried to constant weight at 55° C. in a vacuum oven.

### Example 9

### Synthesis of a Polyether-Urethane-Urea Prepared from Aliphatic and Aromatic Diisocyanates

For an initial charge, poly(tetramethylene)glycol (average Mn=2,900, 168.0 grams, 0.057931 moles) and poly(ethylene glycol-block-propylene glycol-block-ethylene glycol) (average Mn=14,600, 72.0 grams, 0.0049315 moles) are added to a 2000 mL resin reaction kettle that is fitted with a three-neck glass lid equipped with a stainless steel stirrer. The contents are heated to 100° C. at a reduced pressure of less than 0.5 mm Hg to remove moisture. Upon drying, the system is purged with nitrogen gas and cooled to room temperature. Approximately 560 mL of N,N-dimethylacetamide are added to the reaction kettle through a glass funnel to dissolve the dried reaction components. The contents are stirred gently for at least 30 minutes in order to create a homogeneous solution. Hexamethylene diisocyanate (0.0471469 moles, 7.9301 grams) and 4,4′-methylenediphenyl diisocyanate (0.0471469 moles, 250.25 g/mol, 11.7985 grams) are then added, followed by 15 minutes of stirring. Then tin(II) 2-ethyl hexanoate (0.2 M in dioxane, 5.9243 mL, 0.0011849 moles) is added, followed by an additional 15 minutes of stirring, all at room temperature. The contents are then heated to 100° C. and these reaction conditions are maintained for 1.25 hours or until suitable conversion has been attained, and then the temperature is decreased to 25° C. At 25° C. the prepolymer is chain extended by the addition of ethylene diamine (Mn=60.1, 0.031431 moles, 1.8890 grams) while stirring vigorously. The polymer solution is stirred until it has become too viscous to continue stirring. The reaction is allowed to stand overnight at room temperature, and then the polymer is extracted the following day with deionized water for 24 hours followed by acetone for at least 1 hour to remove any unreacted monomer and to deactivate unreacted isocyanate end groups. The purified polymer is isolated and dried to constant weight at 55° C. in a vacuum oven.

### Example 10

### Synthesis of a Polyether-Urethane-Urea Prepared from Aliphatic and Aromatic Diisocyanates and Chain Extended with a Polyether Amine(2,2′-(Ethylenedioxy)Bis(Ethylamine))

For an initial charge, poly(tetramethylene)glycol (average Mn=2,900, 168.0 grams, 0.057931 moles) and poly(ethylene glycol-block-propylene glycol-block-ethylene glycol) (average Mn=14,600, 72.0 grams, 0.0049315 moles) are added to a 2000 mL resin reaction kettle that is fitted with a three-neck glass lid equipped with a stainless steel stirrer. The contents are heated to 100° C. at a reduced pressure of less than 0.5 mm Hg to remove moisture. Upon drying, the system is purged with nitrogen gas and cooled to room temperature. Approximately 560 mL of N,N-dimethylacetamide are added to the reaction kettle through a glass funnel to dissolve the dried reaction components. The contents are stirred gently for at least 30 minutes in order to create a homogeneous solution. Hexamethylene diisocyanate (0.0471469 moles, 7.9301 grams) and 4,4′-methylenediphenyl diisocyanate (0.0471469 moles, 250.25 g/mol, 11.7985 grams) are then added, followed by 15 minutes of stirring. Then tin(II) 2-ethyl hexanoate (0.2 M in dioxane, 5.9243 mL, 0.0011849 moles) is added, followed by an additional 15 minutes of stirring, all at room temperatures. The contents are then heated to 100° C. and these reaction conditions are maintained for 1.25 hours or until suitable conversion has been attained, and then the temperature is decreased to 25° C. At 25° C. the prepolymer is chain extended by the addition of 2,2′-(ethylenedioxy)bis(ethylamine) (MW=148.20, 0.031431 moles, 4.6581 grams) while stirring vigorously. The polymer solution is stirred until it has become too viscous to continue stirring. The reaction is allowed to stand overnight at room temperature, and then the polymer is extracted the following day with deionized water for 24 hours followed by acetone for at least 1 hour to remove any unreacted monomer and to deactivate unreacted isocyanate end groups. The purified polymer is isolated and dried to constant weight at 55° C. in a vacuum oven.

### Example 11

### Synthesis of a Polyether-Carbonate-Urethane-Urea that is Chain Extended with Ethylene Diamine

For an initial charge, poly(hexamethylene-carbonate)diol (average Mn=1000, 180.0 grams, 0.18 moles) and poly(ethylene glycol-block-propylene glycol-block-ethylene glycol) (average Mn=14,600, 20.0 grams, 0.00137) are added to a 2000 mL resin reaction kettle that is fitted with a three-neck glass lid equipped with a stainless steel stirrer. The contents are heated to 100° C. at a reduced pressure of less than 0.5 mm Hg to remove moisture. Upon drying, the system is purged with nitrogen gas and cooled to room temperature. Approximately 560 mL of N,N-dimethylacetamide are added to the reaction kettle through a glass funnel to dissolve the dried reaction components. The contents are stirred gently for at least 30 minutes in order to create a homogeneous solution, followed by addition of hexamethylene diisocyanate (45.76 grams, 0.272055 moles). After stirring for 15 minutes, tin(II) 2-ethyl hexanoate (0.2 M in dioxane, 5.9243 mL, 0.0011849 moles) is added, and the resulting mixture is stirred for 15 minutes, all at room temperature. The contents are then heated to 100° C., and these reaction conditions are maintained for 1.25 hours or until the vessel contents are too thick to continue stirring. Upon obtaining suitable molecular weight, stirring is stopped and the temperature is decreased from 100° C. to room temperature. At 25° C. the prepolymer is chain extended by the addition of ethylene diamine (0.090685 moles, 5.45 grams) while stirring vigorously. The polymer solution is stirred until it becomes too viscous to continue stirring. The reaction is allowed to stand overnight at room temperature, and then the polymer is extracted the following day with deionized water for 24 hours followed by acetone for at least 1 hour to remove any unreacted monomer and to deactivate unreacted isocyanate end groups. The purified polymer is isolated and dried to constant weight at 55° C. in a vacuum oven.

### Example 12

### Synthesis of a Typical Polyether-Urethane-Urea

For an initial charge, poly(tetramethylene)glycol (average Mn=2,900, 168.0 grams, 0.057931 moles) and poly(ethylene glycol-block-propylene glycol-block-ethylene glycol) (average Mn=14,600, 72.0 grams, 0.0049315 moles) are added to a 2000 mL resin reaction kettle that is fitted with a three-neck glass lid equipped with a stainless steel stirrer. The contents are heated to 100° C. at a reduced pressure of less than 0.5 mm Hg to remove moisture. Upon drying, the system is purged with nitrogen gas and cooled to room temperature. Approximately 560 mL of N,N-dimethylacetamide are added to the reaction kettle through a glass funnel to dissolve the dried reaction components. The contents are stirred gently for at least 30 minutes in order to create a homogeneous solution. Hexamethylene diisocyanate (15.86 grams, 0.0942938 moles) is added followed by 15 minutes of stirring. Then tin(II) 2-ethyl hexanoate (0.2 M in dioxane, 5.9243 mL, 0.0011849 moles) is added, followed by an additional 15 minutes of stirring. The contents are then heated to 100° C. and these reaction conditions are maintained for about 1.25 hours. Upon obtaining suitable conversion, the temperature is decreased to room temperature. At room temperature, the prepolymer is chain extended by the addition of ethylene diamine (MW=60.1, 0.031431 moles, 1.8890 grams) while stirring vigorously. The contents are stirred at room temperature until the reaction contents are too viscous to continue stirring. The reaction is allowed to stand overnight at room temperature. The final polymer is extracted with deionized water for 24 hours followed by acetone for at least 1 hour to remove any unreacted monomer and to deactivate unreacted isocyanate end groups. The purified polymer is isolated and dried to constant weight at 55° C. in a vacuum oven. This particular composition will be referred to herein as PEUU-1.

### Example 13

### Synthesis of a Typical Polyether-Urethane-Urea

For an initial charge, poly(tetramethylene)glycol (average Mn=2,900, 192.0 grams, 0.066207 moles, 80% by weight of soft segment) and poly(ethylene glycol-block-propylene glycol-block-ethylene glycol) (20% by weight of soft segment, average Mn=14,600, 48.0 grams, 0.003288 moles) are added to a 2000 mL resin reaction kettle that is fitted with a three-neck glass lid equipped with a stainless steel stirrer. The contents are heated to 100° C. at a reduced pressure of less than 0.5 mm Hg to remove moisture. Upon drying, the system is purged with nitrogen gas and cooled to room temperature. Approximately 560 mL of N,N-dimethylacetamide are added to the reaction kettle through a glass funnel to dissolve the dried reaction components. The contents are stirred gently for at least 30 minutes in order to create a homogeneous solution. Hexamethylene diisocyanate (17.53 grams, 0.104242 moles) is then added, followed by 15 minutes of stirring. To the resulting mixture is added tin(II) 2-ethyl hexanoate (0.2 M in dioxane, 5.9243 mL, 0.0011849 moles) followed by 15 minutes of stirring. The contents are then heated to 100° C. and these reaction conditions are maintained for about 1.25 hours. Upon obtaining suitable conversion, the temperature is decreased to room temperature. At room temperature, the prepolymer is chain extended by the addition of ethylene diamine (MW=60.1, 0.034747 moles, 2.0883 grams) while stirring vigorously. The contents are stirred at room temperature until the reaction contents are too viscous to continue stirring. The reaction is allowed to stand overnight at room temperature. The final polymer is extracted with deionized water for 24 hours followed by acetone for at least 1 hour to remove any unreacted monomer and to deactivate unreacted isocyanate end groups. The purified polymer is isolated and dried to constant weight at 55° C. in a vacuum oven. This particular composition will be referred to herein as PEUU-2.

### Example 14

### Synthesis of a Typical Polyether-Urethane-Urea

For an initial charge, poly(tetramethylene)glycol (average Mn=2,900, 223.2 grams, 0.076966 moles, 93% by weight of soft segment) and poly(ethylene glycol-block-propylene glycol-block-ethylene glycol) (average Mn=14,600, 16.8 grams, 0.001151 moles, 7% by weight of soft segment) are added to a 2000 mL resin reaction kettle that is fitted with a three-neck glass lid equipped with a stainless steel stirrer. The contents are heated to 100° C. at a reduced pressure of less than 0.5 mm Hg to remove moisture. Upon drying, the system is purged with nitrogen gas and cooled to room temperature. Approximately 560 mL of N,N-dimethylacetamide are added to the reaction kettle through a glass funnel to dissolve the dried reaction components. The contents are stirred gently for at least 30 minutes in order to create a homogeneous solution, followed by addition of hexamethylene diisocyanate (19.71 grams, 0.117174 moles). After stirring the resulting mixture for 15 minutes, tin(II) 2-ethyl hexanoate (0.2 M in dioxane, 5.9243 mL, 0.0011849 moles) is added followed by an additional 15 minutes of stirring. The contents are then heated to 100° C., and these reaction conditions are maintained for about 1.25 hours. Upon obtaining suitable conversion, the temperature is decreased to room temperature. At room temperature, the prepolymer is chain extended by the addition of ethylene diamine (MW=60.1, 0.039058 moles, 2.3474 grams) while stirring vigorously. The contents are stirred at room temperature until the reaction contents are too viscous to continue stirring. The reaction is allowed to stand overnight at room temperature. The final polymer is extracted with deionized water for 24 hours followed by acetone for at least 1 hour to remove any unreacted monomer and to deactivate unreacted isocyanate end groups. The purified polymer is isolated and dried to constant weight at 55° C. in a vacuum oven. This particular composition will be referred to herein as PEUU-3.

### Example 15

### Synthesis of a Typical Polyether-Carbonate-Urethane-Urea that is Chain Extended with an Aliphatic Diamine

For an initial charge, poly(hexamethylene-carbonate)diol (average Mn=1000, 57.931 grams, 0.057931 moles) and poly(ethylene glycol-block-propylene glycol-block-ethylene glycol) (average Mn=14,600, 72.0 grams, 0.0049315 moles) are added to a 2000 mL resin reaction kettle that is fitted with a three-neck glass lid equipped with a stainless steel stirrer. The contents are heated to 100° C. at a reduced pressure of less than 0.5 mm Hg to remove moisture. Upon drying, the system is purged with nitrogen gas and cooled to room temperature. Approximately 560 mL of N,N-dimethylacetamide are added to the reaction kettle through a glass funnel to dissolve the dried reaction components. The contents are stirred gently for at least 30 minutes in order to create a homogeneous solution. Hexamethylene diisocyanate (15.86 grams, 0.0942938 moles) is added followed by 15 minutes of stirring, and then tin(II) 2-ethyl hexanoate (0.2 M in dioxane, 5.9243 mL, 0.0011849 moles) is added followed by another 15 minutes of stirring. The contents are then heated to 100° C., and these reaction conditions are maintained for 1.25 hours or until the vessel contents are too thick to continue stirring. Upon obtaining suitable molecular weight, stirring is stopped and the temperature is decreased from 100° C. to room temperature. At 25° C. the prepolymer is chain extended by the addition of ethylene diamine (Mn=60.1, 0.031431 moles, 1.8890 grams) while stirring vigorously. The polymer solution is stirred until it becomes too viscous to continue stirring. The reaction is allowed to stand overnight at room temperature, and then the polymer is extracted the following day with deionized water for 24 hours followed by acetone for at least 1 hour to remove any unreacted monomer and to deactivate unreacted isocyanate end groups. The purified polymer is isolated and dried to constant weight at 55° C. in a vacuum oven. This particular composition will be referred to herein as PEUU-4.

### Example 16

### Synthesis of a Polyether-Urethane-Urea Prepared from a Random Copolymer of Polyethylene Glycol and Polypropylene Glycol and Chain Extended with Hexamethylene Diisocyanate and Ethylene Diamine

The present Example illustrates how a polyether-urethane-urea may be prepared under relatively low temperature conditions compared to, for example, the process disclosed in Example. 1. The present Example is particularly suited for achieving the prepolymer reaction at reduced temperature.

For an initial charge, poly(tetramethylene ether)glycol (average Mn=2,900, 168.0 grams, 0.057931 moles) and poly(ethylene glycol-ran-propylene glycol) (average Mn=12000, 59.178 grams, 0.0049315 moles) are added to a 2000 mL resin reaction kettle that is fitted with a three-neck glass lid equipped with a stainless steel stirrer. The contents are heated to 100° C. at a reduced pressure of less than 0.5 mm Hg to remove moisture. Upon drying, the system is purged with nitrogen gas and cooled to room temperature. Approximately 560 mL of N,N-dimethylacetamide are added to the reaction kettle through a glass funnel to dissolve the dried reaction components. The contents are stirred gently for at least 30 minutes in order to create a homogeneous solution. Then hexamethylene diisocyanate (15.86 grams, 0.0942938 moles)) is added to the solution at room temperature and stirred for 30 minutes. Then tin(II) 2-ethyl hexanoate ((0.2 M in dioxane, 5.9243 mL, 0.0011849 moles)) is added to the reaction mixture and the contents are stirred for 15 minutes. The reaction temperature is increased to 50° C., and the contents are stirred for 2 hours or until suitable conversion has been attained. The prepolymer is then chain extended by the addition of ethylene diamine (Mn=60.1, 0.031431 moles, 1.8890 grams) while stirring vigorously. The polymer solution is stirred until it has become too viscous to continue stirring, and the temperature is decreased to room temperature. The reaction is allowed to stand overnight at room temperature, and then the polymer is extracted the following day with deionized water for 24 hours followed by acetone for at least 1 hour. The purified polymer is isolated and dried to constant weight at 55° C. in a vacuum oven.

### Example 17

### Synthesis of a Polyether-Urethane-Urea Prepared from a Random Copolymer of Polyethylene Glycol and Polypropylene Glycol and Chain Extended with Hexamethylene Diisocyanate and Ethylene Diamine

The present Example illustrates how a polyether-urethane-urea may be prepared under relatively low temperature conditions and in the absence of solvent. The present Example is particularly suited for achieving the prepolymer reaction at reduced temperature and without the use of solvent.

For an initial charge, poly(tetramethylene ether)glycol (average Mn=2,900, 168.0 grams, 0.057931 moles) and poly(ethylene glycol-ran-propylene glycol) (average Mn=12000, 59.178 grams, 0.0049315 moles) are added to a 2000 mL resin reaction kettle that is fitted with a three-neck glass lid equipped with a stainless steel stirrer. The contents are heated to 100° C. at a reduced pressure of less than 0.5 mm Hg to remove moisture. Upon drying, the system is purged with nitrogen gas and cooled to room temperature. The contents are stirred gently for at least 30 minutes in order to create a homogeneous solution. Hexamethylene diisocyanate (15.86 grams, 0.0942938 moles)) is added to the solution at room temperature and stirred for 30 minutes. Tin(II) 2-ethyl hexanoate ((0.2 M in dioxane, 5.9243 mL, 0.0011849 moles)) is then added to the reaction and the contents are heated to 50° C. and stirred for 2 hours or until suitable conversion has been attained. Approximately 560 mL of N,N-dimethylacetamide are added to the reaction kettle through a glass funnel to dissolve the prepolymer. The prepolymer is then chain extended by the addition of ethylene diamine (Mn=60.1, 0.031431 moles, 1.8890 grams) while stirring vigorously. The polymer solution is stirred until it has become too viscous to continue stirring. The reaction is allowed to stand overnight at room temperature, and then the polymer is extracted the following day with deionized water for 24 hours followed by acetone for at least 1 hour. The purified polymer is isolated and dried to constant weight at 55° C. in a vacuum oven.

### Example 18

### Process for Preparation of a Femoral Cap by Dip Coating Method (Single Component)

A polymeric solution consisting of 6% (wt/vol) PEUU-1 (Example 12) in trifluoroethanol is prepared with 12.0 grams of polymer and 200 mL of solvent. The solution is stirred for 16 hours at room temperature until all the polymer has dissolved and the solution is homogeneous. A Teflon mold of the desired femoral cap device is dipped into the polymer solution and placed on a rotating apparatus to rotate the mold as the solvent evaporates. After 30 minutes, the dipping process is repeated. The dipping/drying cycle is continued until the appropriate thickness has been obtained. The mold is rotated overnight for at least 16 hours to allow for further evaporation of solvent in a chemical fume hood, and then the mold is soaked in deionized water to swell the polymer on the surface of the mold. After at least 4 hours of soaking at room temperature in water, the polymer cap is removed from the mold by carefully sliding the cap off of the Teflon mold.

### Example 19

### Process for Preparation of a Multi-Layered Femoral Cap by a Dip Coating Method (3-Layer: Deep, Middle, and Superficial) Using Multiple PEUU Components for Modifying the Hydrophilic Character of Different Layers

A polymeric solution containing 6% by weight PEUU-3 (Example 14) is prepared by dissolution of said polymer in trifluoroethanol. The solution is prepared by dissolving 12.0 grams of PEUU-3 in 200 mL of trifluoroethanol. The solution is stirred for 16 hours at room temperature until all the polymer has dissolved and the solution is homogeneous. In order to prepare the polymeric femoral cap, a Teflon mold with the dimensions of the desired femoral cap device is carefully dipped into the polymer solution. The mold is slowly removed from the polymer solution and attached to a rotating apparatus that rotates the mold as the solvent evaporates. After 30 minutes, the dipping process is repeated for 9 additional cycles. A second polymer solution is prepared with trifluoroethanol, wherein the solution contains 6% by weight of PEUU-1 (Example 12) dissolved in trifluoroethanol. The second solution is used to coat the Teflon mold for 15 additional cycles, using the same dip coating process that is described above. Finally, a third solution is prepared with trifluoroethanol using PEUU-2 (Example 13). This third solution contains 6% by weight of polymer dissolved in trifluoroethanol. This solution is used to coat the Teflon mold for 5 additional cycles in order to attain the desired thickness for the final femoral cap. The mold is rotated overnight for at least 16 hours to allow for further evaporation of solvent in a chemical fume hood. Then, the mold is soaked in deionized water to swell the polymer on the surface of the mold. After at least 4 hours of soaking at room temperature in water, the polymeric cap is removed from the mold by carefully sliding the cap off of the Teflon mold. This particular coating sequence creates a polymeric cap with three distinct layers, wherein the middle layer is the most hydrophilic and the deepest layer is the least hydrophilic.

### Example 20

### Alternative Process for Preparation of a Multi-Layered Femoral Cap by a Dip Coating Method (3-Layer: Deep, Middle, and Superficial) Using Multiple PEUU Components for Modifying Compression Modulus

A polymeric solution containing 6% by weight of a PEUU with high compression modulus is prepared by dissolution of said polymer in trifluoroethanol. The solution is prepared by dissolving 12.0 grams of said PEUU in 200 mL of trifluoroethanol. The solution is stirred for 16 hours at room temperature until all the polymer has dissolved and the solution is homogeneous. In order to prepare the polymeric femoral cap, a Teflon mold with the dimensions of the desired femoral cap device is carefully dipped into the polymer solution. The mold is slowly removed from the polymer solution and attached to a rotating apparatus that rotates the mold as the solvent evaporates. After 30 minutes, the dipping process is repeated for 9 additional cycles. A second polymer solution is prepared with trifluoroethanol, wherein the solution contains 6% by weight of a second PEUU (possessing slightly lower compression modulus than the first PEUU, and also slightly more hydrophilic character) dissolved in trifluoroethanol. The second solution is used to coat the Teflon mold for 15 additional cycles, using the same dip coating process that is described above. Finally, a third solution is prepared with trifluoroethanol using a third PEUU (possessing the lowest compression modulus of the three PEUU components, and also having the greatest hydrophilic character). This third solution contains 6% by weight of polymer dissolved in trifluoroethanol. This solution is used to coat the Teflon mold for 5 additional cycles in order to attain the desired thickness for the final femoral cap. The mold is rotated overnight for at least 16 hours to allow for further evaporation of solvent in a chemical fume hood. Then, the mold is soaked in deionized water to swell the polymer on the surface of the mold. After at least 4 hours of soaking at room temperature in water, the polymeric cap is removed from the mold by carefully sliding the cap off of the Teflon mold. This particular coating sequence creates a polymeric cap with three distinct layers, wherein the hydrophilicity (and associated water content upon swelling) increases from the interior layer to the outermost layer, and the compression modulus decreases from the interior layers to the exterior layers. The outermost layer serves as the articulating surface for the final device, and as such requires greater hydrophilic properties and preferably better orientation of polymer chains parallel to the surface of the femoral cap in order to provide an effective articulating surface.

### Example 21

### Preparation of a Multi-Layered Femoral Cap with Deep, Middle, and Superficial Layers Using an Inversion Technique

A polymeric solution containing 6% by weight PEUU-1 (Example 12) is prepared by dissolution of said polymer in trifluoroethanol. The solution is prepared by dissolving 12.0 grams of PEUU-1 in 200 mL of trifluoroethanol. The solution is stirred for 16 hours at room temperature until all the polymer has dissolved and the solution is homogeneous. In order to prepare the polymeric femoral cap, a Teflon mold with the dimensions of the desired femoral cap device is carefully dipped into the polymer solution. The mold is slowly removed from the polymer solution and attached to a rotating apparatus that rotates the mold as the solvent evaporates. After 30 minutes, the dipping process is repeated for 5 additional cycles. The mold is again placed on the rotating apparatus to allow the solvent to evaporate over a 16 hour period. The polymeric cap is carefully removed from the mold, and then the cap is turned inside out and placed back onto the Teflon mold. A second polymer solution is prepared with trifluoroethanol, wherein the solution contains 6% by weight of PEUU-2 (Example 13) dissolved in trifluoroethanol. The second solution is used to coat the Teflon mold for 15 additional cycles, using the same dip coating process that is described above. Finally, a third solution is prepared with trifluoroethanol using PEUU-3 (Example 14). This third solution contains 6% by weight of polymer dissolved in trifluoroethanol. This solution is used to coat the Teflon mold for 9 additional cycles in order to attain the desired thickness for the final femoral cap. The mold is rotated overnight for at least 16 hours to allow for further evaporation of solvent in a chemical fume hood. Then, the mold is soaked in deionized water to swell the polymer on the surface of the mold. After at least 4 hours of soaking at room temperature in water, the polymeric cap is removed from the mold by carefully sliding the cap off of the Teflon mold. The cap is then carefully removed from the mold, and the cap is inverted again to yield the polymeric cap in the correct orientation. This particular coating sequence creates a polymeric cap with three distinct layers, wherein the hydrophilicity (and associated water content upon swelling) increases from the interior layer to the outermost layer. Furthermore, this method creates a pre-stressed superficial layer in the femoral cap as a result of coating the superficial layer on the mold in the first step prior to inversion.

### Example 22

### Preparation of a Zonally Differentiated, Multi-Layered Femoral Cap by Dip-Coating and Compression Molding

A preform of a femoral cap is prepared by a dip-coating method as described in Example 16, above, using 6 cycles of dip coating, and wherein the constituent polymer is PEUU-1 (Example 12). The preform is compression molded using a 30 ton Carver press. The cap is then removed from the mold, inverted, and placed back onto the mold. The mold is dipped into a new polymer solution containing 6% by weight of PEUU-2 (Example 13) dissolved in trifluoroethanol. The mold is placed on the rotating device and allowed to dry for 30 minutes. Then, the dip coating process is repeated for an additional 15 cycles. The mold is then dipped into a third polymer solution containing 6% by weight of PEUU-3 (Example 14) dissolved in trifluoroethanol. The mold is rotated for 30 minutes as the solvent evaporates, and then the dip coating process is repeated with the same polymer solution for an additional 8 cycles in order to attain the desired thickness. The mold is rotated overnight for at least 16 hours to allow for further evaporation of solvent in a chemical fume hood. Then, the mold is soaked in deionized water to swell the polymer on the surface of the mold. After at least 4 hours of soaking at room temperature in water, the polymeric cap is removed from the mold by carefully sliding the cap off of the Teflon mold. The cap is then carefully removed from the mold, and the cap is inverted again to yield the polymeric cap in the correct orientation.

### Example 23

### Process for Preparation of Femoral Cap by Dip-Coating Process and Intra-Layer Chemical Reactions

A polymeric solution consisting of 6% by weight of PEUU-3 (Example 14) in trifluoroethanol is prepared with 12.0 grams of polymer and 200 mL of solvent. The solution is stirred for 16 hours at room temperature until all the polymer has dissolved and the solution is homogeneous. A Teflon mold of the desired femoral cap device is then dipped into the polymer solution and placed on a rotating apparatus to rotate the mold as the solvent evaporates. After 30 minutes, the dipping process is repeated. The dip coating is repeated for an additional 10 cycles. An outer layer is then applied by dipping the mold into a second solution containing 5% by weight of a diisocyanate-capped polyurethane dissolved in N,N-dimethylacetamide. The mold is rotated for 5 minutes, and then the mold is dipped into a third solution containing 5% by weight of a second prepolymer with diamine end groups. The mold is then rotated at room temperature for 30 minutes and the dipping process is continued. The mold is dipped into a solution containing 6% by weight of PEUU1 (Example 12) dissolved in trifluoroethanol. The mold is rotated overnight for at least 16 hours to allow for further evaporation of solvent in a chemical fume hood, and then the mold is soaked in deionized water to swell the polymer on the surface of the mold. After at least 4 hours of soaking at room temperature in water, the polymer cap is removed from the mold by carefully sliding the cap off of the Teflon® polymer mold.

### Example 24

### Preparation of a Multi-Layered Femoral Cap by Dip-Coating and Intra-Layer Chemical Reaction

A femoral cap consisting of a polyether urethane urea (PEUU) interior and a polyether carbonate urethane urea outer surface, wherein the two layers are chemically fused by a reaction between diisocyanate and diamine functional groups. This cap is prepared using a 6% by weight solution of PEUU-1 (Example 12), in which the solution is prepared by dissolution in trifluoroethanol, using 12.0 grams of polymer and 200 mL of solvent. The solution is stirred for 16 hours at room temperature until all the polymer has dissolved and the solution is homogeneous. A Teflon mold of the desired femoral cap device is then dipped into the polymer solution and placed on a rotating apparatus to rotate the mold as the solvent evaporates. After 30 minutes, the dipping process is repeated. The dip coating process is repeated for an additional 10 cycles. An outer layer is then applied by dipping the mold into a second solution containing 5% by weight of a diisocyanate-capped polyurethane. The mold is rotated for 5 minutes, and then the mold is dipped into a third solution containing 5% by weight of a second prepolymer with diamine end groups, wherein said prepolymer consists of polycarbonate and polyether segments that are interlinked with diisocyanate. The mold is then rotated at room temperature for 30 minutes and the dipping process is continued. A new solution containing 5% by weight of PECUU-1 in trifluoroethanol is used to coat the outer layers of the mold. The mold is dipped into the solution of PECUU-1, and then the mold is rotated at room temperature for 30 minutes to allow the solvent to dry. Two more layers of PECUU-1 are applied to the outer surface of the mold, and then the mold is rotated overnight for at least 16 hours to allow for further evaporation of solvent in a chemical fume hood. The mold is soaked in deionized water to swell the polymer cap, and after at least 4 hours of soaking at room temperature in water, the polymer cap is removed from the mold by carefully sliding the cap off of the Teflon mold.

### Example 25

### Preparation of a Femoral Cap Using Medical Imagining Techniques to Determine Dimensions for Femoral Cap Mold

The diseased joint that is to be treated with the femoral cap device is imaged by MRI or CAT scan to determine the exact dimensions of the bones that constitute the joint. These dimensions are reduced by at least 10% and used to create a custom Teflon mold in the shape of the desired femoral cap. The smaller dimensions are used because it creates a tighter fit of the final device on the femoral head. Upon the preparation of the custom mold, a polymeric solution consisting of PEUU-1 in trifluoroethanol is used to coat the mold and prepare a femoral cap, using the method as described in Example 16.

### Example 26

### Preparation of a Femoral Cap Using a Three-Dimensional Printing Machine

The diseased joint that is to be treated with the femoral cap device is imaged by MRI or CAT scan to determine the exact dimensions of the bones that constitute the joint. These dimensions become the input for the three-dimensional polymer printing machine. A solution of PEUU-1 (Example 12) dissolved in trifluoroethanol is loaded in the printer, and the femoral cap is prepared by printing of the polymer in a layer-by-layer fashion using the dimensions obtained from imaging of the joint. When printing is complete, the device is allowed to dry in a chemical fume hood so that remaining solvent evaporates. Then the device is moved to a vacuum chamber and dried at reduced pressure for at least 24 hours at room temperature.

### Example 27

### Preparation of a Femoral Cap by a Spray Coating Method

A 1% by weight polymer solution is prepared that consists of 1 gram of PEUU-1 (Example 12) per 100 milliliters of trifluoroethanol. The polymer solution is loaded into a spray bottle that is capable of applying an even coat of polymer through misting of the solution. The solution is applied to the surface of a Teflon mold that is fabricated to the appropriate dimensions for the final device. The solution is sprayed evenly to the surface of the mold, and then the solvent is allowed to dry for 30 minutes while rotating prior to the application of the next layer. The spray coating process is continued until the appropriate thickness is attained. Upon completion of the spray coating process, the mold is rotated in a chemical fume hood for at least 16 hours to allow for the evaporation of solvent to continue. Next, the mold is placed in a vacuum chamber and dried at reduced pressure for at least 24 hours at room temperature. The mold is removed from the vacuum chamber and soaked in deionized water for at least 4 hours to swell the polymeric cap, and then the cap is carefully removed from the mold.

### Example 28

### Preparation of a Femoral Cap by Dip Coating and Application of Bioactive Agents to Interior Layers

A 6% by weight solution of PEUU-1 (Example 12) is prepared by dissolution in trifluoroethanol, using 12.0 grams of polymer and 200 mL of solvent. The solution is stirred for 16 hours at room temperature until all the polymer has dissolved and the solution is homogeneous. A Teflon mold in the shape of the desired femoral cap device is then dipped into the polymer solution and carefully removed. The mold is placed on a rotating apparatus to in order to continually rotate the mold as the solvent evaporates. After 30 minutes, the dipping process is repeated. The dipping/drying cycle is continued for 4 additional cycles, and then a solution containing PEUU-1 and a bioactive agent is used to dip coat the mold in order to apply the bioactive agent. The mold is allowed to dry for 30 minutes, and then the dip coating process is continued for 20 more cycles using a fresh solution of 6% PEUU-1 in trifluoroethanol. Then a second solution containing a different bioactive agent is sprayed onto the surface of the mold in order to apply said bioactive agent. The mold is allowed to dry for 30 minutes, and then the dip coating process is continued for 5 more cycles using a fresh solution of 6% PEUU-1 in trifluoroethanol to attain the appropriate thickness for the final device. The mold is rotated overnight for at least 16 hours to allow for further evaporation of solvent in a chemical fume hood, and then the polymeric cap is carefully removed from the mold.

### Example 29

### Preparation of a Femoral Cap by Dip Coating and Application of Bioactive Agents to Interior Layers

A 6% by weight solution of PEUU-1 (Example 12) is prepared by dissolution in trifluoroethanol, using 12.0 grams of polymer and 200 mL of solvent. The solution is stirred for 16 hours at room temperature until all the polymer has dissolved and the solution is homogeneous. A Teflon mold in the shape of the desired femoral cap device is then dipped into the polymer solution and carefully removed. The mold is placed on a rotating apparatus to in order to continually rotate the mold as the solvent evaporates. After 30 minutes, the dipping process is repeated. The dipping/drying cycle is continued for 4 additional cycles. Next, the mold is dipped into a solution containing PEUU-1 and a bioactive agent that is dissolved in trifluoroethanol. The mold is allowed to dry for 30 minutes, and then the dip coating process is continued for 20 more cycles using a fresh solution of 6% PEUU-1 in trifluoroethanol. Then the mold is dipped into a second solution containing a different bioactive agent in order to apply said bioactive agent. The mold is allowed to dry for 30 minutes, and then the dip coating process is continued for 5 more cycles using a fresh solution of 6% PEUU-1 in trifluoroethanol to attain the appropriate thickness for the final device. The mold is rotated overnight for at least 16 hours to allow for further evaporation of solvent in a chemical fume hood, and then the mold is soaked in deionized water to swell the polymer on the surface of the mold. After at least 4 hours of soaking at room temperature in water, the polymeric cap is carefully removed from the mold.

### Example 30

### Swelling of Femoral Cap Using Drug-Loaded Solution

Prior to implantation through a surgical procedure, the polymeric femoral cap in its unhydrated form is soaked in an aqueous solution containing a bioactive agent. The femoral cap is completely submerged in the solution for at least 6 hours so that the polymer can become hydrated and swell with said bioactive agent. After 6 hours of soaking in the methyl cellulose solution, the polymer femoral cap is removed from the solution and ready for implantation.

### Example 31

### Alternative Method for Preparation of a Femoral Cap from Thin Films

A polymeric solution containing 6% by weight of PEUU-1 (Example 12) dissolved in trifluoroethanol is prepared by dissolving 6 grams of polymer in 100 milliliters of solvent. When the polymer is fully dissolved, the solution is cast into a tray measuring 10 centimeters by 10 centimeters and allowed to dry in a chemical fume hood while the tray is covered. The solution is allowed to dry for at least 48 hours to ensure that enough solvent has evaporated. The film is then removed from the tray and heated to 100° C. for 10 minutes. A Teflon mold in the shape of the femoral cap device is then pressed into the hot film so that the polymer encompasses the exterior of the mold and conforms to its shape. The mold and polymer are allowed to cool to room temperature, and then the excess polymer is trimmed from the lower stem of the mold. The mold is then soaked in deionized water for 4 hours, and then the polymeric cap is carefully removed.

### Example 32

### Process for Preparation of a Multi-Layered Femoral Cap by a Dip Coating Method (3-Layer: Deep, Middle, and Superficial) Using Multiple PEUU Components for Modifying the Hydrophilic Character of Different Layers

A polymeric solution containing 6% by weight PEUU-3 (Example 14) is prepared by dissolution of said polymer in trifluoroethanol. The solution is prepared by dissolving 12.0 grams of PEUU-3 in 200 mL of trifluoroethanol. The solution is stirred for 16 hours at room temperature until all the polymer has dissolved and the solution is homogeneous. In order to prepare the polymeric femoral cap, a Teflon mold with the dimensions of the desired femoral cap device is carefully dipped into the polymer solution. The mold is slowly removed from the polymer solution and attached to a rotating apparatus that rotates the mold as the solvent evaporates. After 30 minutes, the dipping process is repeated for 9 additional cycles. A second polymer solution is prepared with trifluoroethanol, wherein the solution contains 6% by weight of PEUU-2 (Example 2) dissolved in trifluoroethanol. The second solution is used to coat the Teflon mold for 15 additional cycles, using the same dip coating process that is described above. Finally, a third solution is prepared with trifluoroethanol using PEUU-1 (Example 12). This third solution contains 6% by weight of polymer dissolved in trifluoroethanol. This solution is used to coat the Teflon mold for 5 additional cycles in order to attain the desired thickness for the final femoral cap. The mold is rotated overnight for at least 16 hours to allow for further evaporation of solvent in a chemical fume hood. Then, the mold is soaked in deionized water to swell the polymer on the surface of the mold. After at least 4 hours of soaking at room temperature in water, the polymeric cap is removed from the mold by carefully sliding the cap off of the Teflon mold. This particular coating sequence creates a polymeric cap with three distinct layers, wherein the outer layer is the most hydrophilic and the deepest layer is the least hydrophilic.

### Example 33

### Alternative Process for Preparation of a Multi-Layered Femoral Cap by a Dip Coating Method (3-Layer: Deep, Middle, and Superficial) Using Multiple PEUU Components for Modifying Compression Modulus

A polymeric solution containing 6% by weight of a PEUU with high compression modulus is prepared by dissolution of said polymer in trifluoroethanol. The solution is prepared by dissolving 12.0 grams of said PEUU in 200 mL of trifluoroethanol. The solution is stirred for 16 hours at room temperature until all the polymer has dissolved and the solution is homogeneous. In order to prepare the polymeric femoral cap, a Teflon mold with the dimensions of the desired femoral cap device is carefully dipped into the polymer solution. The mold is slowly removed from the polymer solution and attached to a rotating apparatus that rotates the mold as the solvent evaporates. After 30 minutes, the dipping process is repeated for 9 additional cycles. A second polymer solution is prepared with trifluoroethanol, wherein the solution contains 6% by weight of a second PEUU (possessing slightly lower compression modulus than the first PEUU, and also slightly more hydrophilic character) dissolved in trifluoroethanol. The second solution is used to coat the Teflon mold for 15 additional cycles, using the same dip coating process that is described above. Finally, a third solution is prepared with trifluoroethanol using a third PEUU (possessing the lowest compression modulus of the three PEUU components, and also having the greatest hydrophilic character). This third solution contains 6% by weight of polymer dissolved in trifluoroethanol. This solution is used to coat the Teflon mold for 5 additional cycles in order to attain the desired thickness for the final femoral cap. The mold is rotated overnight for at least 16 hours to allow for further evaporation of solvent in a chemical fume hood. Then, the mold is soaked in deionized water to swell the polymer on the surface of the mold. After at least 4 hours of soaking at room temperature in water, the polymeric cap is removed from the mold by carefully sliding the cap off of the Teflon mold. This particular coating sequence creates a polymeric cap with three distinct layers, wherein the percent water absorption increases from the interior layer to the surface layer, and the compression modulus decreases gradually from the interior layer to the surface layer. The surface layer serves as the articulating surface for the final device, and as such requires greater hydrophilic properties and preferably better orientation of polymer chains parallel to the surface of the femoral cap in order to provide an effective articulating surface.

### Example 34

### Multi-Layered Femoral Cap by a Dip Coating Method (3-Layer: Deep, Middle, and Superficial) Using Multiple PEUU Components for Modifying Compression Modulus

A multilayer composition may be prepared as described herein where PEUU-1 (Example 12) is used in the preparation of the surface layer, PEUU-2 (example 12) is used in the middle layer, and PEUU-3 (Example 14) is used in the interior-most layer.

The various embodiments described above can be combined to provide further embodiments. Aspects of the embodiments can be modified, if necessary to employ concepts of the various patents, applications and publications to provide yet further embodiments. The following numbered embodiments are exemplary of the embodiments of the present disclosure:

1) A multilayer composition comprising a first layer in contact with a second layer, the first layer comprising a first polymer and the second layer comprising a second polymer, the first polymer comprising a plurality of urethane or urea groups, the second polymer comprising a plurality of urethane or urea groups, the first and second polymers being non-identical.

2) The composition of embodiment 1 wherein the first polymer further comprises a plurality of urea groups.

3) The composition of embodiment 1 wherein the first polymer further comprises a plurality of ether groups.

4) The composition of embodiment 1 wherein the first polymer further comprises a plurality of carbonate groups.

5) The composition of embodiment 1 wherein the first layer is more hydrophilic than the second layer.

6) The composition of embodiment 1 wherein the first layer is less hydrophilic than the second layer.

7) The composition of embodiment 1 wherein the first layer is more elastic than the second layer.

8) The composition of embodiment 1 wherein the first layer has a greater hardness than the second layer.

9) The composition of embodiment 1 wherein the first layer has a higher surface modulus than the second layer.

10) The composition of embodiment 1 wherein the first layer has a greater toughness than the second layer.

11) The composition of embodiment 1 wherein the first layer has a higher Tg than the second layer.

12) The composition of embodiment 1 wherein the first layer has a lower Tg than the second layer.

13) The composition of embodiment 1 wherein the first layer has a thickness of 0.1 to 1.5 mm.

14) The composition of embodiment 1 wherein the first layer has a thickness of 0.1 to 1.0 mm.

15) The composition of embodiment 1 wherein the first layer has a thickness of 0.1 to 0.5 mm.

16) The composition of embodiment 1 having exactly two layers.

17) The composition of embodiment 1 further comprising a third layer in contact with the second layer, the third layer comprising a third polymer, the third polymer comprising a plurality of urethane groups, the second and third polymers being non-identical.

18) The composition of embodiment 17, having exactly three layers.

19) The composition of embodiment 17, the first and third polymers being identical

20) The composition of embodiment 17, the first and third polymers being non-identical.

21) The composition of embodiment 17, wherein the second layer is more hydrophilic than either the first layer or the third layer.

22) The composition of embodiment 17, wherein the second layer is more hydrophilic than the first layer.

23) The composition of embodiment 17, where the second polymer contains a greater weight percent of ether linkages compared to the first polymer, and wherein the second polymer is more hydrophilic than either the first polymer or the third polymer.

24) The composition of embodiment 17, where the second polymer contains a greater weight percent of ether linkages compared to the first polymer, and wherein the second polymer is more hydrophilic than the first polymer.

25) The composition of embodiment 17, wherein the second layer is more elastic than either the first layer or the third layer.

26) The composition of embodiment 17, wherein the second layer is more elastic than the first layer.

27) The composition of embodiment 17, wherein the first layer has a greater hardness than the second layer.

28) The composition of embodiment 17, wherein the first layer has a higher surface modulus than the second layer.

29) The composition of embodiment 17, wherein the first layer has a greater toughness than the second layer.

30) The composition of embodiment 1 wherein the first layer contains a pharmaceutically active agent.

31) The composition of embodiment 1 wherein the second layer contains a pharmaceutically active agent.

32) The composition of embodiment 1 having a thickness of 1.0 to 3.0 mm

33) The composition of embodiment 1 having a thickness of 1.5 to 2.0 mm

34) The composition of embodiment 1 which is marked.

35) The composition of embodiment 1 which is sterile.

36) The composition of embodiment 1 which has a longest straight line dimension of 1 to 5 cm or is in the form of a cap having a half-circumference of 10-200 mm.

37) The composition of any of embodiments 1-25 wherein one or both of the first and second polymers is the reaction product of a pre-polymer and a diamine, where the pre-polymer is the reaction product of a diisocyanate and a polyetherdiol.

38) The composition of embodiment 37 wherein the polyetherdiol comprises at least one type of oxyalkylene sequence selected from the group consisting of oxyethylene, oxypropylene, oxytrimethylene and oxytetramethylene sequences.

39) The composition of any of embodiments 37-38 wherein the polyetherdiol is a blend of polyetherdiols.

40) The composition of any of embodiments 37-38 wherein the polyether diol is not a blend of polyetherdiols.

41) The composition of any of embodiments 37-40 wherein the polyetherdiol is a random copolymer of two or more oxyalkylene sequences.

42) The composition of any of embodiments 37-40 wherein the polyetherdiol is a block copolymer of two or more oxyalkylene sequences.

43) The composition of any of embodiments 37-40 wherein the polyetherdiol is an alternating copolymer of two or more oxyalkylene sequences.

44) The composition of any of embodiments 37-40 wherein the polyetherdiol is an alternating copolymer of two different oxyalklylene repeat units, for example, oxyethylene and oxypropylene.

45) The composition of any of embodiments 37-42 wherein the diamine is an aliphatic diamine.

46) The composition of any of embodiments 37-42 wherein the diamine is a polyether diamine.

47) The composition of any of embodiments 37-42 wherein the diamine is a blend of diamines.

48) The composition of any of embodiments 37-42 wherein the diamine is a blend of aliphatic diamine and polyether diamine.

49) The composition of any of embodiments 1-25 wherein one or both of the first and second polymers is the reaction product of diisocyanate and a polyetherdiamine to form a pre-polymer, and the reaction product of the pre-polymer and a diol to form a polyether urea urethane.

50) The composition of embodiment 49 wherein the polyetherdiamine comprises at least one type of oxyalkylene sequence selected from the group consisting of oxyethylene, oxypropylene, oxytrimethylene and oxytetramethylene sequences.

51) The composition of any of embodiments 49 or 50 wherein the polyetherdiamine is a blend of polyetherdiamines

52) The composition of any of embodiments 49 or 50 wherein the polyetherdiamine is not a blend of polyetherdiamines.

53) The composition of any of embodiments 49-52 wherein the polyetherdiamine is a random copolymer of two or more oxyalkylene sequences.

54) The composition of any of embodiments 49-52 wherein the polyetherdiamine is a block copolymer of two or more oxyalkylene sequences.

55) The composition of any of embodiments 49-54 wherein the diol is an aliphatic diol.

56) The composition of any of embodiments 49-54 wherein the diol is an aromatic diol.

57) The composition of any of embodiments 49-54 wherein the diol is a polyether diol.

58) The composition of any of embodiments 49-54 wherein the diol is a blend of diols.

59) The composition of any of embodiments 49-54 wherein the diol is a blend of aliphatic diol and polyetherdiol.

60) The composition of any of embodiments 37-59 wherein the diisocyanate is an aliphatic diisocyanate and the reactants do not include an aromatic diisocyanate to form a polyether urea urethane.

61) The composition of any of embodiments 37-59 wherein the diisocyanate is a mixture of aliphatic diisocyanates and the reactants do not include an aromatic diisocyanate to form a polyether urea urethane.

62) The composition of any of embodiments 37-59 wherein the diisocyanate is an aromatic diisocyanate and the reactants do not include an aliphatic diisocyanate to form a polyether urea urethane.

63) The composition of any of embodiments 37-59 wherein the diisocyanate is a mixture of aromatic diisocyanates and the reactants do not include an aliphatic diisocyanate to form a polyether urea urethane.

64) The composition of any of embodiments 37-59 wherein the diisocyanate is a mixture of aromatic diisocyanate and aliphatic diisocyanate to form a polyether urea urethane.

65) The composition of any of embodiments 1-25 wherein one or both of the first and second polymers is the reaction product of a diisocyanate and a diol.

66) The composition of embodiment 65 wherein the diol is a polyether diol.

67) The composition of embodiment 66 wherein the polyether diol comprises at least one type of oxyalkylene sequence selected from the group consisting of oxyethylene, oxypropylene, oxytrimethylene and oxytetramethylene sequences.

68) The composition of any of embodiments 65 or 66 wherein the polyetherdiol is a blend of polyetherdiols.

69) The composition of any of embodiments 65 or 66 wherein the polyether diol is not a blend of polyetherdiols.

70) The composition of any of embodiments 66-69 wherein the polyetherdiol is a random copolymer of two or more oxyalkylene sequences.

71) The composition of any of embodiments 66-69 wherein the polyetherdiol is a block copolymer of two or more oxyalkylene sequences.

72) The composition of any of embodiments 65-71 wherein the diisocyanate is an aliphatic diisocyanate and the reactants do not include an aromatic diisocyanate.

73) The composition of any of embodiments 65-71 wherein the diisocyanate is a mixture of aliphatic diisocyanates and the reactants do not include an aromatic diisocyanate.

74) The composition of any of embodiments 65-71 wherein the diisocyanate is an aromatic diisocyanate and the reactants do not include an aliphatic diisocyanate.

75) The composition of any of embodiments 65-71 wherein the diisocyanate is a mixture of aromatic diisocyanates and the reactants do not include an aliphatic diisocyanate.

76) The composition of any of embodiments 65-71 wherein the diisocyanate is a mixture of aromatic diisocyanate and aliphatic diisocyanate.

77) The composition of any of embodiments 65-76 wherein diisocyanate and diol are the only reactants.

78) The composition of any of embodiments 65-77 wherein the molar ratio of diisocyanate to polyether diol is in the range of 0.95 to 1.05.

79) The composition of any of embodiments 1-25 wherein one or both of the first and second polymers is the reaction product of a diisocyanate and a diamine.

80) The composition of embodiment 79 wherein the diamine is a polyether diamine.

81) The composition of embodiment 80 wherein the polyether diamine comprises at least one type of oxyalkylene sequence selected from the group consisting of oxyethylene, oxypropylene, oxytrimethylene and oxytetramethylene sequences.

82) The composition of any of embodiments 79 or 80 wherein the polyetherdiamine is a blend of polyetherdiamines

83) The composition of any of embodiments 79 or 80 wherein the polyether diamine is not a blend of polyetherdiamines.

84) The composition of any of embodiments 80-83 wherein the polyetherdiamine is a random copolymer of two or more oxyalkylene sequences.

85) The composition of any of embodiments 80-83 wherein the polyetherdiamine is a block copolymer of two or more oxyalkylene sequences.

86) The composition of any of embodiments 79-85 wherein the diisocyanate is an aliphatic diisocyanate and the reactants do not include an aromatic diisocyanate.

87) The composition of any of embodiments 79-85 wherein the diisocyanate is a mixture of aliphatic diisocyanates and the reactants do not include an aromatic diisocyanate.

88) The composition of any of embodiments 79-85 wherein the diisocyanate is an aromatic diisocyanate and the reactants do not include an aliphatic diisocyanate.

89) The composition of any of embodiments 79-85 wherein the diisocyanate is a mixture of aromatic diisocyanates and the reactants do not include an aliphatic diisocyanate.

90) The composition of any of embodiments 79-85 wherein the diisocyanate is a mixture of aromatic diisocyanate and aliphatic diisocyanate.

91) The composition of any of embodiments 79-90 wherein diisocyanate and diamine are the only reactants.

92) The composition of any of embodiments 80-91 wherein the molar ratio of diisocyanate to polyether diamine is in the range of 0.95 to 1.05.

93) The composition of any of embodiments 1-25 wherein one or both of the first and second polymers is the reaction product of a diisocyanate and either (a) a mixture comprising polyether diol and polycarbonate diol or (b) a polyether polycarbonate diol.

94) The composition of embodiment 93 wherein the polyetherdiol comprises at least one type of oxyalkylene sequence selected from the group consisting of oxyethylene, oxypropylene, oxytrimethylene and oxytetramethylene sequences.

95) The composition of any of embodiments 93 or 94 wherein the polyetherdiol is a blend of polyetherdiols.

96) The composition of any of embodiments 93 or 94 wherein the polyether diol is not a blend of polyetherdiols.

97) The composition of any one of embodiments 93-96 wherein the polyetherdiol is a random copolymer of two or more oxyalkylene sequences.

98) The composition of any one of embodiments 93-96 wherein the polyetherdiol is a block copolymer of two or more oxyalkylene sequences.

99) The composition of any of embodiments 93-96 wherein the polyetherdiol is an alternating copolymer of two or more oxyalkylene sequences.

100) The composition of any of embodiments 93-96 wherein the polyetherdiol is an alternating copolymer of two different oxyalklylene repeat units, for example, oxyethylene and oxypropylene.

101) The composition of any one of embodiments 93-100 wherein the polycarbonate diol is poly(hexamethylene carbonate)diol.

102) The composition of any one of embodiments 93-100 wherein the polycarbonate diol is poly(ethylene-carbonate)diol.

103) The composition of any one of embodiments 93-100 wherein the polycarbonate diol is the reaction product of trimethylene carbonate and a diol.

104) The composition of any one of embodiments 93-103 wherein the diisocyanate is an aliphatic diisocyanate and the reactants do not include an aromatic diisocyanate.

105) The composition of any one of embodiments 93-103 wherein the diisocyanate is a mixture of aliphatic diisocyanates and the reactants do not include an aromatic diisocyanate.

106) The composition of any one of embodiments 93-103 wherein the diisocyanate is an aromatic diisocyanate and the reactants do not include an aliphatic diisocyanate.

107) The composition of any one of embodiments 93-103 wherein the diisocyanate is a mixture of aromatic diisocyanates and the reactants do not include an aliphatic diisocyanate.

108) The composition of any one of embodiments 93-103 wherein the diisocyanate is a mixture of aromatic diisocyanate and aliphatic diisocyanate.

109) The composition of any one of embodiments 93-108 which is further chain extended by reaction with a diol.

110) The composition of any of embodiments 1-25 wherein one or both of the first and second polymers is the reaction product of a diamine and a pre-polymer, where the pre-polymer is the reaction product of a diisocyanate and either (a) a mixture comprising polyether diol and polycarbonate diol or (b) a polyether polycarbonate diol.

111) The composition of embodiment 110 wherein the polyetherdiol comprises at least one type of oxyalkylene sequence selected from the group consisting of oxyethylene, oxypropylene, oxytrimethylene and oxytetramethylene sequences.

112) The composition of any of embodiments 110 or 111 wherein the polyetherdiol is a blend of polyetherdiols.

113) The composition of any of embodiments 110 or 111 wherein the polyether diol is not a blend of polyetherdiols.

114) The composition of any one of embodiments 110-113 wherein the polyetherdiol is a random copolymer of two or more oxyalkylene sequences.

115) The composition of any one of embodiments 110-113 wherein the polyetherdiol is a block copolymer of two or more oxyalkylene sequences.

116) The composition of any of embodiments 110-113 wherein the polyetherdiol is an alternating copolymer of two or more oxyalkylene sequences.

117) The composition of any of embodiments 110-113 wherein the polyetherdiol is an alternating copolymer of two different oxyalklylene repeat units, for example, oxyethylene and oxypropylene.

118) The composition of any one of embodiments 110-117 wherein the polycarbonate diol is poly(hexamethylene carbonate)diol.

119) The composition of any one of embodiments 110-117 wherein the polycarbonate diol is poly(ethylene-carbonate)diol.

120) The composition of any one of embodiments 110-117 wherein the polycarbonate diol is the reaction product of trimethylene carbonate and a diol.

121) The composition of any one of embodiments 110-120 wherein the diisocyanate is an aliphatic diisocyanate and the reactants do not include an aromatic diisocyanate.

122) The composition of any one of embodiments 110-120 wherein the diisocyanate is a mixture of aliphatic diisocyanates and the reactants do not include an aromatic diisocyanate.

123) The composition of any one of embodiments 110-120 wherein the diisocyanate is an aromatic diisocyanate and the reactants do not include an aliphatic diisocyanate.

124) The composition of any one of embodiments 110-120 wherein the diisocyanate is a mixture of aromatic diisocyanates and the reactants do not include an aliphatic diisocyanate.

125) The composition of any one of embodiments 110-120 wherein the diisocyanate is a mixture of aromatic diisocyanate and aliphatic diisocyanate.

126) The composition of any one of embodiments 110-125 wherein the diamine is an aliphatic diamine.

127) The composition of any one of embodiments 110-125 wherein the diamine is a polyether diamine.

128) The composition of any one of embodiments 110-125 wherein the diamine is a blend of diamines

129) The composition of any one of embodiments 110-125 wherein the diamine is a blend of aliphatic diamine and polyether diamine.

130) The composition of any of embodiments 1-25 wherein one or both of the first and second polymer is the reaction product of a diisocyanate and either (a) a mixture comprising polyether diol and polyester diol or (b) a polyether polyester diol.

131) The composition of embodiment 130 wherein the polyether diol comprises at least one type of oxyalkylene sequence selected from the group consisting of oxyethylene, oxypropylene, oxytrimethylene and oxytetramethylene sequences.

132) The composition of any of embodiments 130 or 131 wherein the polyether diol is a blend of polyetherdiols.

133) The composition of any of embodiments 130 or 131 wherein the polyether diol is not a blend of polyetherdiols.

134) The composition of any one of embodiments 130-133 wherein the polyether diol is a random copolymer of two or more oxyalkylene sequences.

135) The composition of any one of embodiments 130-133 wherein the polyether diol is a block copolymer of two or more oxyalkylene sequences.

136) The composition of any of embodiments 130-133 wherein the polyetherdiol is an alternating copolymer of two or more oxyalkylene sequences.

137) The composition of any of embodiments 130-133 wherein the polyetherdiol is an alternating copolymer of two different oxyalklylene repeat units, for example, oxyethylene and oxypropylene.

138) The composition of any one of embodiments 130-137 wherein the diisocyanate is an aliphatic diisocyanate and the reactants do not include an aromatic diisocyanate.

139) The composition of any one of embodiments 130-137 wherein the diisocyanate is a mixture of aliphatic diisocyanates and the reactants do not include an aromatic diisocyanate.

140) The composition of any one of embodiments 130-137 wherein the diisocyanate is an aromatic diisocyanate and the reactants do not include an aliphatic diisocyanate.

141) The composition of any one of embodiments 130-137 wherein the diisocyanate is a mixture of aromatic diisocyanates and the reactants do not include an aliphatic diisocyanate.

142) The composition of any one of embodiments 130-137 wherein the diisocyanate is a mixture of aromatic diisocyanate and aliphatic diisocyanate.

143) The composition of any of embodiments 1-25 wherein one or both of the first and second polymer is the reaction product of a diamine and a pre-polymer, where the pre-polymer is the reaction product of a diisocyanate and either (a) a mixture comprising polyether diol and polyester diol or (b) a polyether polyester diol.

144) The composition of embodiment 143 wherein the polyetherdiol comprises at least one type of oxyalkylene sequence selected from the group consisting of oxyethylene, oxypropylene, oxytrimethylene and oxytetramethylene sequences.

145) The composition of one of embodiments 143 or 144 wherein the polyetherdiol is a blend of polyetherdiols.

146) The composition of any one of embodiments 143 or 144 wherein the polyether diol is not a blend of polyetherdiols.

147) The composition of any one of embodiments 143-146 wherein the polyetherdiol is a random copolymer of two or more oxyalkylene sequences.

148) The composition of any one of embodiments 143-146 wherein the polyetherdiol is a block copolymer of two or more oxyalkylene sequences.

149) The composition of any of embodiments 143-146 wherein the polyetherdiol is an alternating copolymer of two or more oxyalkylene sequences.

150) The composition of any of embodiments 143-146 wherein the polyetherdiol is an alternating copolymer of two different oxyalklylene repeat units, for example, oxyethylene and oxypropylene.

151) The composition of any one of embodiments 143-150 wherein the diisocyanate is an aliphatic diisocyanate and the reactants do not include an aromatic diisocyanate.

152) The composition of any one of embodiments 143-150 wherein the diisocyanate is a mixture of aliphatic diisocyanates and the reactants do not include an aromatic diisocyanate.

153) The composition of any one of embodiments 143-150 wherein the diisocyanate is an aromatic diisocyanate and the reactants do not include an aliphatic diisocyanate.

154) The composition of any one of embodiments 143-150 wherein the diisocyanate is a mixture of aromatic diisocyanates and the reactants do not include an aliphatic diisocyanate.

155) The composition of any one of embodiments 143-150 wherein the diisocyanate is a mixture of aromatic diisocyanate and aliphatic diisocyanate.

156) The composition of any one of embodiments 143-155 wherein the diamine is an aliphatic diamine.

157) The composition of any one of embodiments 143-155 wherein the diamine is a polyether diamine.

158) The composition of any one of embodiments 143-155 wherein the diamine is a blend of diamines

159) The composition of any one of embodiments 143-155 wherein the diamine is a blend of aliphatic diamine and polyether diamine.

160) The composition any of one of embodiments 1-159 wherein at least one of the first and second polymer is bio-stable.

161) The composition any of one of embodiments 1-159 wherein at least one of the first and second polymers absorbs at least 50% of its weight in water when immersed in 1% aqueous methyl cellulose at 37° C. for 16 hours.

162) The composition any of one of embodiments 1-159 wherein at least one of the first and second polymers has a COF of 0.001 to 0.15.

163) The composition any of one of embodiments 1-159 wherein at least one of the first and second polymers has an intrinsic viscosity of 3-8 dl/g.

164) A multilayer composition in the form of a cap, the cap comprising a center and a rim that surrounds the center, at least one of the center and the rim comprising a first layer in contact with a second layer, the first layer comprising a first polymer and the second layer comprising a second polymer, the first polymer comprising a plurality of urethane or urea groups, the second polymer comprising a plurality of urethane or urea groups, the first and second polymers being non-identical.

165) The composition of embodiment 164 wherein the center and the rim have the same number of layers.

166) The composition of embodiment 164 wherein the center and the rim have a different number of layers.

167) The composition of embodiment 164 having a half-circumference of 10 to 200 mm.

168) The composition of embodiment 164 wherein the center has a thickness from 1.5 to 2.0 mm and the rim has a thickness from 0.5 to 1.5 mm.

169) The composition of embodiment 164 wherein the center and rim each has a thickness, where the center thickness is greater than the rim thickness.

170) The composition of embodiment 164 wherein the rim comprises at least a first layer and the center comprises at least a first layer and a second layer.

171) The composition of embodiment 164 wherein the rim comprises at least a first and second layer and the center comprises at least a first, second and third layer.

172) The composition of any of embodiments 164-171 wherein the first polymer further comprises a plurality of urea groups.

173) The composition of any of embodiments 164-171 wherein the first polymer further comprises a plurality of ether groups.

174) The composition of any of embodiments 164-171 wherein the first polymer further comprises a plurality of carbonate groups.

175) The composition of any of embodiments 164-171 wherein the first layer is more hydrophilic than the second layer.

176) The composition of any of embodiments 164-171 wherein the first layer is less hydrophilic than the second layer.

177) The composition of any of embodiments 164-171 wherein the first layer is more elastic than the second layer.

178) The composition of any of embodiments 164-171 wherein the first layer is less elastic than the second layer.

179) The composition of any of embodiments 164-171 wherein the first layer has a greater hardness than the second layer.

180) The composition of any of embodiments 149-156 wherein the first layer has a higher surface modulus than the second layer.

181) The composition of any of embodiments 164-171 wherein the first layer has a greater toughness than the second layer.

182) The composition of any of embodiments 164-171 wherein the first layer has a higher Tg than the second layer.

183) The composition of any of embodiments 164-171 wherein the first layer has a lower Tg than the second layer.

184) The composition of any of embodiments 164-171 wherein the first layer has a thickness of 0.1 to 1.5 mm.

185) The composition of any of embodiments 164-171 wherein the first layer has a thickness of 0.1 to 1.0 mm.

186) The composition of any of embodiments 164-171 wherein the first layer has a thickness of 0.1 to 0.5 mm.

187) The composition of any of embodiments 164-171 having exactly two layers.

188) The composition of any of embodiments 164-171 further comprising a third layer in contact with the second layer, the third layer comprising a third polymer, the third polymer comprising a plurality of urethane groups, the second and third polymers being non-identical.

189) The composition of embodiment 188, wherein at least one of the cap and the rim has exactly three layers.

190) The composition of embodiment 188, the first and third polymers being identical

191) The composition of embodiment 188, the first and third polymers being non-identical.

192) The composition of embodiment 188, wherein the second layer is more hydrophilic than either the first layer or the third layer.

193) The composition of embodiment 188, wherein the second layer is less hydrophilic than the third layer.

194) The composition of claim 188, wherein the second layer is more hydrophilic than the first layer.

195) The composition of any of embodiments 164-192, wherein the second polymer contains a greater weight percent of ether linkages compared to the first polymer, and wherein the second polymer is more hydrophilic than either the first polymer or the third polymer.

196) The composition of any of embodiments 164-192, wherein the second polymer contains a greater weight percent of ether linkages compared to the first polymer, and wherein the second polymer is more hydrophilic than the first polymer.

197) The composition of any of embodiments 164-192, wherein the second layer is more elastic than either the first layer or the third layer.

198) The composition of any of embodiments 164-192, wherein the second layer is more elastic than the first layer.

199) The composition of any of embodiments 164-192, wherein the first layer has a greater hardness than the second layer.

200) The composition of any of embodiments 164-192, wherein the first layer has a higher surface modulus than the second layer.

201) The composition of any of embodiments 164-192, wherein the first layer has a greater toughness than the second layer.

202) The composition of any of embodiments 164-192, wherein the second layer has a greater toughness than the first layer.

203) The composition of any of embodiments 164-202 wherein the first layer contains a pharmaceutically active agent.

204) The composition of any of embodiments 164-203 wherein the second layer contains a pharmaceutically active agent.

205) The composition of any of embodiments 164-204 having a thickness of 1.0 to 3.0 mm.

206) The composition of any of embodiments 164-204 having a thickness of 1.5 to 2.0 mm.

207) The composition of any of embodiments 164-204 which is marked.

208) The composition of any of embodiments 164-204 which is sterile.

209) The composition of any of embodiments 164-204 which has a longest straight line dimension of 1 to 5 cm or is in the form of a cap having a half-circumference of 10-200 mm.

210) The composition of any of embodiments 164-209 wherein one or both of the first and second polymer is the reaction product of a pre-polymer and a diamine, where the pre-polymer is the reaction product of a diisocyanate and a polyetherdiol.

211) The composition of embodiment 210 wherein the polyetherdiol comprises at least one type of oxyalkylene sequence selected from the group consisting of oxyethylene, oxypropylene, oxytrimethylene and oxytetramethylene sequences.

212) The composition of any of embodiments 210 or 211 wherein the polyetherdiol is a blend of polyetherdiols.

213) The composition of any of embodiments 210 or 211 wherein the polyether diol is not a blend of polyetherdiols.

214) The composition of any of embodiments 210-213 wherein the polyetherdiol is a random copolymer of two or more oxyalkylene sequences.

215) The composition of any of embodiments 210-213 wherein the polyetherdiol is a block copolymer of two or more oxyalkylene sequences.

216) The composition of any of embodiments 210-213 wherein the polyetherdiol is an alternating copolymer of two or more oxyalkylene sequences.

217) The composition of any of embodiments 210-213 wherein the polyetherdiol is an alternating copolymer of two different oxyalklylene repeat units, for example, oxyethylene and oxypropylene.

218) The composition of any of embodiments 210-217 wherein the diamine is an aliphatic diamine.

219) The composition of any of embodiments 210-217 wherein the diamine is a polyether diamine.

220) The composition of any of embodiments 210-217 wherein the diamine is a blend of diamines.

221) The composition of any of embodiments 210-217 wherein the diamine is a blend of aliphatic diamine and polyether diamine.

222) The composition of any of embodiments 164-209 wherein one or both of the first and second polymer is the reaction product of diisocyanate and a polyetherdiamine to form a pre-polymer, and the reaction product of the pre-polymer and a diol to form a polyether urea urethane.

223) The composition of embodiment 222 wherein the polyetherdiamine comprises at least one type of oxyalkylene sequence selected from the group consisting of oxyethylene, oxypropylene, oxytrimethylene and oxytetramethylene sequences.

224) The composition of any of embodiments 222 or 223 wherein the polyetherdiamine is a blend of polyetherdiamines.

225) The composition of any of embodiments 222 or 223 wherein the polyetherdiamine is not a blend of polyetherdiamines.

226) The composition of any of embodiments 222-225 wherein the polyetherdiamine is a random copolymer of two or more oxyalkylene sequences.

227) The composition of any of embodiments 222-225 wherein the polyetherdiamine is a block copolymer of two or more oxyalkylene sequences.

228) The composition of any of embodiments 222-227 wherein the diol is an aliphatic diol.

229) The composition of any of embodiments 222-227 wherein the diol is an aromatic diol.

230) The composition of any of embodiments 222-227 wherein the diol is a polyether diol.

231) The composition of any of embodiments 222-227 wherein the diol is a blend of diols.

232) The composition of any of embodiments 222-227 wherein the diol is a blend of aliphatic diol and polyetherdiol.

233) The composition of any of embodiments 222-232 wherein the diisocyanate is an aliphatic diisocyanate and the reactants do not include an aromatic diisocyanate to form a polyether urea urethane.

234) The composition of any of embodiments 222-232 wherein the diisocyanate is a mixture of aliphatic diisocyanates and the reactants do not include an aromatic diisocyanate to form a polyether urea urethane.

235) The composition of any of embodiments 222-232 wherein the diisocyanate is an aromatic diisocyanate and the reactants do not include an aliphatic diisocyanate to form a polyether urea urethane.

236) The composition of any of embodiments 222-232 wherein the diisocyanate is a mixture of aromatic diisocyanates and the reactants do not include an aliphatic diisocyanate to form a polyether urea urethane.

237) The composition of any of embodiments 222-232 wherein the diisocyanate is a mixture of aromatic diisocyanate and aliphatic diisocyanate to form a polyether urea urethane.

238) The composition of any of embodiments 164-209 wherein one or both of the first and second polymer is the reaction product of a diisocyanate and a diol.

239) The composition of embodiment 238 wherein the diol is a polyether diol.

240) The composition of embodiment 239 wherein the polyether diol comprises at least one type of oxyalkylene sequence selected from the group consisting of oxyethylene, oxypropylene, oxytrimethylene and oxytetramethylene sequences.

241) The composition of any of embodiments 239 or 240 wherein the polyetherdiol is a blend of polyetherdiols.

242) The composition of any of embodiments 239 or 240 wherein the polyether diol is not a blend of polyetherdiols.

243) The composition of any of embodiments 239-242 wherein the polyetherdiol is a random copolymer of two or more oxyalkylene sequences.

244) The composition of any of embodiments 239-242 wherein the polyetherdiol is a block copolymer of two or more oxyalkylene sequences.

245) The composition of any of embodiments 239-242 wherein the polyetherdiol is an alternating copolymer of two or more oxyalkylene sequences.

246) The composition of any of embodiments 239-242 wherein the polyetherdiol is an alternating copolymer of two different oxyalklylene repeat units, for example, oxyethylene and oxypropylene.

247) The composition of any of embodiments 238-246 wherein the diisocyanate is an aliphatic diisocyanate and the reactants do not include an aromatic diisocyanate.

248) The composition of any of embodiments 238-246 wherein the diisocyanate is a mixture of aliphatic diisocyanates and the reactants do not include an aromatic diisocyanate.

249) The composition of any of embodiments 238-246 wherein the diisocyanate is an aromatic diisocyanate and the reactants do not include an aliphatic diisocyanate.

250) The composition of any of embodiments 238-246 wherein the diisocyanate is a mixture of aromatic diisocyanates and the reactants do not include an aliphatic diisocyanate.

251) The composition of any of embodiments 238-246 wherein the diisocyanate is a mixture of aromatic diisocyanate and aliphatic diisocyanate.

252) The composition of any of embodiments 238-251 wherein diisocyanate and diol are the only reactants.

253) The composition of any of embodiments 238-251 wherein the molar ratio of diisocyanate to polyether diol is in the range of 0.95 to 1.05.

254) The composition of any of embodiments 164-209 wherein one or both of the first and second polymer is the reaction product of a diisocyanate and a diamine.

255) The composition of embodiment 254 wherein the diamine is a polyether diamine.

256) The composition of embodiment 255 wherein the polyether diamine comprises at least one type of oxyalkylene sequence selected from the group consisting of oxyethylene, oxypropylene, oxytrimethylene and oxytetramethylene sequences.

257) The composition of any of embodiments 255 or 256 wherein the polyetherdiamine is a blend of polyetherdiamines.

258) The composition of any of embodiments 255 or 256 wherein the polyether diamine is not a blend of polyetherdiamines.

259) The composition of any of embodiments 255-258 wherein the polyetherdiamine is a random copolymer of two or more oxyalkylene sequences.

260) The composition of any of embodiments 255-258 wherein the polyetherdiamine is a block copolymer of two or more oxyalkylene sequences.

261) The composition of any of embodiments 255-260 wherein the diisocyanate is an aliphatic diisocyanate and the reactants do not include an aromatic diisocyanate.

262) The composition of any of embodiments 255-260 wherein the diisocyanate is a mixture of aliphatic diisocyanates and the reactants do not include an aromatic diisocyanate.

263) The composition of any of embodiments 255-260 wherein the diisocyanate is an aromatic diisocyanate and the reactants do not include an aliphatic diisocyanate.

264) The composition of any of embodiments 255-260 wherein the diisocyanate is a mixture of aromatic diisocyanates and the reactants do not include an aliphatic diisocyanate.

265) The composition of any of embodiments 255-260 wherein the diisocyanate is a mixture of aromatic diisocyanate and aliphatic diisocyanate.

266) The composition of any of embodiments 255-265 wherein diisocyanate and diamine are the only reactants.

267) The composition of any of embodiments 255-265 wherein the molar ratio of diisocyanate to polyether diamine is in the range of 0.95 to 1.05.

268) The composition of any of embodiments 164-209 wherein one or both of the first and second polymer is the reaction product of a diisocyanate and either (a) a mixture comprising polyether diol and polycarbonate diol or (b) a polyether polycarbonate diol.

269) The composition of embodiment 268 wherein the polyetherdiol comprises at least one type of oxyalkylene sequence selected from the group consisting of oxyethylene, oxypropylene, oxytrimethylene and oxytetramethylene sequences.

270) The composition of any of embodiments 268 or 269 wherein the polyetherdiol is a blend of polyetherdiols.

271) The composition of any of embodiments 268 or 269 wherein the polyether diol is not a blend of polyetherdiols.

272) The composition of any one of embodiments 268-271 wherein the polyetherdiol is a random copolymer of two or more oxyalkylene sequences.

273) The composition of any one of embodiments 268-271 wherein the polyetherdiol is a block copolymer of two or more oxyalkylene sequences.

274) The composition of any of embodiments 268-271 wherein the polyetherdiol is an alternating copolymer of two or more oxyalkylene sequences.

275) The composition of any of embodiments 268-271 wherein the polyetherdiol is an alternating copolymer of two different oxyalklylene repeat units, for example, oxyethylene and oxypropylene.

276) The composition of any one of embodiments 268-275 wherein the polycarbonate diol is poly(hexamethylene carbonate)diol.

277) The composition of any one of embodiments 268-275 wherein the polycarbonate diol is poly(ethylene-carbonate)diol.

278) The composition of any one of embodiments 268-275 wherein the polycarbonate diol is the reaction product of trimethylene carbonate and a diol.

279) The composition of any one of embodiments 268-278 wherein the diisocyanate is an aliphatic diisocyanate and the reactants do not include an aromatic diisocyanate.

280) The composition of any one of embodiments 268-278 wherein the diisocyanate is a mixture of aliphatic diisocyanates and the reactants do not include an aromatic diisocyanate.

281) The composition of any one of embodiments 268-278 wherein the diisocyanate is an aromatic diisocyanate and the reactants do not include an aliphatic diisocyanate.

282) The composition of any one of embodiments 268-278 wherein the diisocyanate is a mixture of aromatic diisocyanates and the reactants do not include an aliphatic diisocyanate.

283) The composition of any one of embodiments 268-278 wherein the diisocyanate is a mixture of aromatic diisocyanate and aliphatic diisocyanate.

284) The composition of any one of embodiments 268-278 which is further chain extended by reaction with a diol.

285) The composition of any of embodiments 164-209 wherein one or both of the first and second polymer is the reaction product of a diamine and a pre-polymer, where the pre-polymer is the reaction product of a diisocyanate and either (a) a mixture comprising polyether diol and polycarbonate diol or (b) a polyether polycarbonate diol.

286) The composition of embodiment 285 wherein the polyetherdiol comprises at least one type of oxyalkylene sequence selected from the group consisting of oxyethylene, oxypropylene, oxytrimethylene and oxytetramethylene sequences.

287) The composition of any of embodiments 285 or 286 wherein the polyetherdiol is a blend of polyetherdiols.

288) The composition of any of embodiments 285 or 286 wherein the polyether diol is not a blend of polyetherdiols.

289) The composition of any one of embodiments 285-288 wherein the polyetherdiol is a random copolymer of two or more oxyalkylene sequences.

290) The composition of any one of embodiments 285-288 wherein the polyetherdiol is a block copolymer of two or more oxyalkylene sequences.

291) The composition of any of embodiments 285-288 wherein the polyetherdiol is an alternating copolymer of two or more oxyalkylene sequences.

292) The composition of any of embodiments 285-288 wherein the polyetherdiol is an alternating copolymer of two different oxyalklylene repeat units, for example, oxyethylene and oxypropylene.

293) The composition of any one of embodiments 285-292 wherein the polycarbonate diol is poly(hexamethylene carbonate)diol.

294) The composition of any one of embodiments 285-292 wherein the polycarbonate diol is poly(ethylene-carbonate)diol.

295) The composition of any one of embodiments 285-292 wherein the polycarbonate diol is the reaction product of trimethylene carbonate and a diol.

296) The composition of any one of embodiments 285-295 wherein the diisocyanate is an aliphatic diisocyanate and the reactants do not include an aromatic diisocyanate.

297) The composition of any one of embodiments 285-295 wherein the diisocyanate is a mixture of aliphatic diisocyanates and the reactants do not include an aromatic diisocyanate.

298) The composition of any one of embodiments 285-295 wherein the diisocyanate is an aromatic diisocyanate and the reactants do not include an aliphatic diisocyanate.

299) The composition of any one of embodiments 285-295 wherein the diisocyanate is a mixture of aromatic diisocyanates and the reactants do not include an aliphatic diisocyanate.

300) The composition of any one of embodiments 285-295 wherein the diisocyanate is a mixture of aromatic diisocyanate and aliphatic diisocyanate.

301) The composition of any one of embodiments 285-300 wherein the diamine is an aliphatic diamine.

302) The composition of any one of embodiments 285-300 wherein the diamine is a polyether diamine.

303) The composition of any one of embodiments 285-300 wherein the diamine is a blend of diamines.

304) The composition of any one of embodiments 285-300 wherein the diamine is a blend of aliphatic diamine and polyether diamine.

305) The composition of any of embodiments 164-209 wherein one or both of the first and second polymer is the reaction product of a diisocyanate and either (a) a mixture comprising polyether diol and polyester diol or (b) a polyether polyester diol.

306) The composition of embodiment 305 wherein the polyether diol comprises at least one type of oxyalkylene sequence selected from the group consisting of oxyethylene, oxypropylene, oxytrimethylene and oxytetramethylene sequences.

307) The composition of any of embodiments 305 or 306 wherein the polyether diol is a blend of polyetherdiols.

308) The composition of any of embodiments 305 or 306 wherein the polyether diol is not a blend of polyetherdiols.

309) The composition of any one of embodiments 305-308 wherein the polyether diol is a random copolymer of two or more oxyalkylene sequences.

310) The composition of any one of embodiments 305-308 wherein the polyether diol is a block copolymer of two or more oxyalkylene sequences.

311) The composition of any of embodiments 305-308 wherein the polyetherdiol is an alternating copolymer of two or more oxyalkylene sequences.

312) The composition of any of embodiments 305-308 wherein the polyetherdiol is an alternating copolymer of two different oxyalklylene repeat units, for example, oxyethylene and oxypropylene.

313) The composition of any one of embodiments 305-312 wherein the diisocyanate is an aliphatic diisocyanate and the reactants do not include an aromatic diisocyanate.

314) The composition of any one of embodiments 305-312 wherein the diisocyanate is a mixture of aliphatic diisocyanates and the reactants do not include an aromatic diisocyanate.

315) The composition of any one of embodiments 305-312 wherein the diisocyanate is an aromatic diisocyanate and the reactants do not include an aliphatic diisocyanate.

316) The composition of any one of embodiments 305-312 wherein the diisocyanate is a mixture of aromatic diisocyanates and the reactants do not include an aliphatic diisocyanate.

317) The composition of any one of embodiments 305-312 wherein the diisocyanate is a mixture of aromatic diisocyanate and aliphatic diisocyanate.

318) The composition of any of embodiments 164-209 wherein one or both of the first and second polymer is the reaction product of a diamine and a pre-polymer, where the pre-polymer is the reaction product of a diisocyanate and either (a) a mixture comprising polyether diol and polyester diol or (b) a polyether polyester diol.

319) The composition of embodiment 318 wherein the polyetherdiol comprises at least one type of oxyalkylene sequence selected from the group consisting of oxyethylene, oxypropylene, oxytrimethylene and oxytetramethylene sequences.

320) The composition of one of embodiments 318 or 319 wherein the polyetherdiol is a blend of polyetherdiols.

321) The composition of any one of embodiments 318 or 319 wherein the polyether diol is not a blend of polyetherdiols.

322) The composition of any one of embodiments 318-321 wherein the polyetherdiol is a random copolymer of two or more oxyalkylene sequences.

323) The composition of any one of embodiments 318-321 wherein the polyetherdiol is a block copolymer of two or more oxyalkylene sequences.

324) The composition of any of embodiments 318-321 wherein the polyetherdiol is an alternating copolymer of two or more oxyalkylene sequences.

325) The composition of any of embodiments 318-321 wherein the polyetherdiol is an alternating copolymer of two different oxyalklylene repeat units, for example, oxyethylene and oxypropylene.

326) The composition of any one of embodiments 318-325 wherein the diisocyanate is an aliphatic diisocyanate and the reactants do not include an aromatic diisocyanate.

327) The composition of any one of embodiments 318-325 wherein the diisocyanate is a mixture of aliphatic diisocyanates and the reactants do not include an aromatic diisocyanate.

328) The composition of any one of embodiments 318-325 wherein the diisocyanate is an aromatic diisocyanate and the reactants do not include an aliphatic diisocyanate.

329) The composition of any one of embodiments 318-325 wherein the diisocyanate is a mixture of aromatic diisocyanates and the reactants do not include an aliphatic diisocyanate.

330) The composition of any one of embodiments 318-325 wherein the diisocyanate is a mixture of aromatic diisocyanate and aliphatic diisocyanate.

331) The composition of any one of embodiments 318-330 wherein the diamine is an aliphatic diamine.

332) The composition of any one of embodiments 318-330 wherein the diamine is a polyether diamine.

333) The composition of any one of embodiments 318-330 wherein the diamine is a blend of diamines.

334) The composition of any one of embodiments 318-330 wherein the diamine is a blend of aliphatic diamine and polyether diamine.

335) The composition any of one of embodiments 164-334 wherein at least one of the first and second polymer is bio-stable.

336) The composition any of one of embodiments 164-334 wherein at least one of the first and second polymers absorbs at least 50% of its weight in water when immersed in 1% aqueous methyl cellulose at 37° C. for 16 hours.

337) The composition any of one of embodiments 164-334 wherein at least one of the first and second polymers has a COF of 0.001 to 0.15.

338) The composition any of one of embodiments 164-334 wherein at least one of the first and second polymers has an intrinsic viscosity of 3-8 dl/g.

339) A composition comprising a cap in combination with a support, the support selected from the group consisting of a bone and a material in the shape of a bone, wherein the cap fits snugly around a portion of the support, and the cap comprises a multilayer composition comprising a first layer in contact with a second layer, the first layer comprising a first polymer and the second layer comprising a second polymer, the first polymer comprising a plurality of urethane or urea groups, the second polymer comprising a plurality of urethane or urea groups, the first and second polymers being non-identical.

340) The composition of embodiment 339 wherein the support is in the shape of a head of a femur bone.

341) The composition of embodiment 339 wherein the support comprises Teflon® or equivalent.

342) The composition of embodiment 339 wherein the center and the rim have the same number of layers.

343) The composition of embodiment 339 wherein the center and the rim have a different number of layers.

344) The composition of embodiment 339 having a half-circumference of 10 to 200 mm.

345) The composition of embodiment 339 wherein the center has a thickness from 1.5 to 2.0 mm and the rim has a thickness from 0.5 to 1.5 mm.

346) The composition of embodiment 339 wherein the center and rim each has a thickness, where the center thickness is greater than the rim thickness.

347) The composition of embodiment 339 wherein the rim comprises at least a first layer and the center comprises at least a first layer and a second layer.

348) The composition of embodiment 339 wherein the rim comprises at least a first and second layer and the center comprises at least a first, second and third layer.

349) The composition of any of embodiments 339-348 wherein the first polymer further comprises a plurality of urea groups.

350) The composition of any of embodiments 339-348 wherein the first polymer further comprises a plurality of ether groups.

351) The composition of any of embodiments 339-348 wherein the first polymer further comprises a plurality of carbonate groups.

352) The composition of any of embodiments 339-348 wherein the first layer is more hydrophilic than the second layer.

353) The composition of any of embodiments 339-348 wherein the first layer is less hydrophilic than the second layer.

354) The composition of any of embodiments 339-348 wherein the first layer is more elastic than the second layer.

355) The composition of any of embodiments 339-348 wherein the first layer is less elastic than the second layer.

356) The composition of any of embodiments 339-348 wherein the first layer has a greater hardness than the second layer.

357) The composition of any of embodiments 339-348 wherein the first layer has a higher surface modulus than the second layer.

358) The composition of any of embodiments 339-348 wherein the first layer has a greater toughness than the second layer.

359) The composition of any of embodiments 339-348 wherein the first layer has a higher Tg than the second layer.

360) The composition of any of embodiments 339-348 wherein the first layer has a lower Tg than the second layer.

361) The composition of any of embodiments 339-348 wherein the first layer has a thickness of 0.1 to 1.5 mm.

362) The composition of any of embodiments 339-348 wherein the first layer has a thickness of 0.1 to 1.0 mm.

363) The composition of any of embodiments 339-348 wherein the first layer has a thickness of 0.1 to 0.5 mm.

364) The composition of any of embodiments 339-348 having exactly two layers.

365) The composition of any of embodiments 339-348 further comprising a third layer in contact with the second layer, the third layer comprising a third polymer, the third polymer comprising a plurality of urethane groups, the second and third polymers being non-identical.

366) The composition of embodiment 365 wherein at least one of the cap and the rim has exactly three layers.

367) The composition of embodiment 365, the first and third polymers being identical

368) The composition of embodiment 365, the first and third polymers being non-identical.

369) The composition of embodiment 365, wherein the second layer is more hydrophilic than either the first layer or the third layer.

370) The composition of embodiment 365, wherein the second layer is more hydrophilic than the first layer.

371) The composition of any of embodiments 339-369, wherein the second polymer contains a greater weight percent of ether linkages compared to the first polymer, and wherein the second polymer is more hydrophilic than either the first polymer or the third polymer.

372) The composition of any of embodiments 339-369, wherein the second polymer contains a greater weight percent of ether linkages compared to the first polymer, and wherein the second polymer is more hydrophilic than the first polymer.

373) The composition of any of embodiments 339-369, wherein the second layer is more elastic than either the first layer or the third layer.

374) The composition of any of embodiments 339-369, wherein the second layer is more elastic than the first layer.

375) The composition of any of embodiments 339-369 wherein the first layer has a higher Tg than the second layer, or alternatively, the first layer has a lower Tg than the second layer.

376) The composition of any of embodiments 339-369, wherein the first layer has a greater hardness than the second layer.

377) The composition of any of embodiments 339-369, wherein the first layer has a higher surface modulus than the second layer.

378) The composition of any of embodiments 339-369, wherein the first layer has a greater toughness than the second layer.

379) The composition of any of embodiments 339-369, wherein the first layer contains a pharmaceutically active agent.

380) The composition of any of embodiments 339-369 wherein the second layer contains a pharmaceutically active agent.

381) The composition of any of embodiments 339-380 having a thickness of 1.0 to 3.0 mm.

382) The composition of any of embodiments 339-380 having a thickness of 1.5 to 2.0 mm.

383) The composition of any of embodiments 339-380 which is marked.

384) The composition of any of embodiments 339-380 which is sterile.

385) The composition of any of embodiments 339-380 which has a longest straight line dimension of 1 to 5 cm or is in the form of a cap having a half-circumference of 10-200 mm.

386) The composition of any of embodiments 339-385 wherein one or both of the first and second polymer is the reaction product of a pre-polymer and a diamine, where the pre-polymer is the reaction product of a diisocyanate and a polyetherdiol.

387) The composition of embodiment 386 wherein the polyetherdiol comprises at least one type of oxyalkylene sequence selected from the group consisting of oxyethylene, oxypropylene, oxytrimethylene and oxytetramethylene sequences.

388) The composition of any of embodiments 386 or 387 wherein the polyetherdiol is a blend of polyetherdiols.

389) The composition of any of embodiments 386 or 387 wherein the polyether diol is not a blend of polyetherdiols.

390) The composition of any of embodiments 386-389 wherein the polyetherdiol is a random copolymer of two or more oxyalkylene sequences.

391) The composition of any of embodiments 386-389 wherein the polyetherdiol is a block copolymer of two or more oxyalkylene sequences.

392) The composition of any of embodiments 386-389 wherein the polyetherdiol is an alternating copolymer of two or more oxyalkylene sequences.

393) The composition of any of embodiments 386-389 wherein the polyetherdiol is an alternating copolymer of two different oxyalklylene repeat units, for example, oxyethylene and oxypropylene.

394) The composition of any of embodiments 386-393 wherein the diamine is an aliphatic diamine.

395) The composition of any of embodiments 386-393 wherein the diamine is a polyether diamine.

396) The composition of any of embodiments 386-393 wherein the diamine is a blend of diamines.

397) The composition of any of embodiments 386-393 wherein the diamine is a blend of aliphatic diamine and polyether diamine.

398) The composition of any of embodiments 339-385 wherein one or both of the first and second polymer is the reaction product of diisocyanate and a polyetherdiamine to form a pre-polymer, and the reaction product of the pre-polymer and a diol to form a polyether urea urethane.

399) The composition of embodiment 398 wherein the polyetherdiamine comprises at least one type of oxyalkylene sequence selected from the group consisting of oxyethylene, oxypropylene, oxytrimethylene and oxytetramethylene sequences.

400) The composition of any of embodiments 398 or 399 wherein the polyetherdiamine is a blend of polyetherdiamines.

401) The composition of any of embodiments 398 or 399 wherein the polyetherdiamine is not a blend of polyetherdiamines.

402) The composition of any of embodiments 398-401 wherein the polyetherdiamine is a random copolymer of two or more oxyalkylene sequences.

403) The composition of any of embodiments 398-401 wherein the polyetherdiamine is a block copolymer of two or more oxyalkylene sequences.

404) The composition of any of embodiments 398-401 wherein the polyetherdiamine is an alternating copolymer of two or more oxyalkylene sequences.

405) The composition of any of embodiments 398-401 wherein the polyetherdiamine is an alternating copolymer of two different oxyalklylene repeat units, for example, oxyethylene and oxypropylene.

406) The composition of any of embodiments 398-405 wherein the diol is an aliphatic diol.

407) The composition of any of embodiments 398-405 wherein the diol is an aromatic diol.

408) The composition of any of embodiments 398-405 wherein the diol is a polyether diol.

409) The composition of any of embodiments 398-405 wherein the diol is a blend of diols.

410) The composition of any of embodiments 398-405 wherein the diol is a blend of aliphatic diol and polyetherdiol.

411) The composition of any of embodiments 398-410 wherein the diisocyanate is an aliphatic diisocyanate and the reactants do not include an aromatic diisocyanate to form a polyether urea urethane.

412) The composition of any of embodiments 398-410 wherein the diisocyanate is a mixture of aliphatic diisocyanates and the reactants do not include an aromatic diisocyanate to form a polyether urea urethane.

413) The composition of any of embodiments 398-410 wherein the diisocyanate is an aromatic diisocyanate and the reactants do not include an aliphatic diisocyanate to form a polyether urea urethane.

414) The composition of any of embodiments 398-410 wherein the diisocyanate is a mixture of aromatic diisocyanates and the reactants do not include an aliphatic diisocyanate to form a polyether urea urethane.

415) The composition of any of embodiments 398-410 wherein the diisocyanate is a mixture of aromatic diisocyanate and aliphatic diisocyanate to form a polyether urea urethane.

416) The composition of any of embodiments 339-385 wherein one or both of the first and second polymer is the reaction product of a diisocyanate and a diol.

417) The composition of embodiment 416 wherein the diol is a polyether diol.

418) The composition of embodiment 417 wherein the polyether diol comprises at least one type of oxyalkylene sequence selected from the group consisting of oxyethylene, oxypropylene, oxytrimethylene and oxytetramethylene sequences.

419) The composition of any of embodiments 417 or 418 wherein the polyetherdiol is a blend of polyetherdiols.

420) The composition of any of embodiments 417 or 418 wherein the polyether diol is not a blend of polyetherdiols.

421) The composition of any of embodiments 417-420 wherein the polyetherdiol is a random copolymer of two or more oxyalkylene sequences.

422) The composition of any of embodiments 417-420 wherein the polyetherdiol is a block copolymer of two or more oxyalkylene sequences.

423) The composition of any of embodiments 417-420 wherein the polyetherdiol is an alternating copolymer of two or more oxyalkylene sequences.

424) The composition of any of embodiments 417-420 wherein the polyetherdiol is an alternating copolymer of two different oxyalklylene repeat units, for example, oxyethylene and oxypropylene.

425) The composition of any of embodiments 417-424 wherein the diisocyanate is an aliphatic diisocyanate and the reactants do not include an aromatic diisocyanate.

426) The composition of any of embodiments 417-424 wherein the diisocyanate is a mixture of aliphatic diisocyanates and the reactants do not include an aromatic diisocyanate.

427) The composition of any of embodiments 417-424 wherein the diisocyanate is an aromatic diisocyanate and the reactants do not include an aliphatic diisocyanate.

428) The composition of any of embodiments 417-424 wherein the diisocyanate is a mixture of aromatic diisocyanates and the reactants do not include an aliphatic diisocyanate.

429) The composition of any of embodiments 417-424 wherein the diisocyanate is a mixture of aromatic diisocyanate and aliphatic diisocyanate.

430) The composition of any of embodiments 417-429 wherein diisocyanate and diol are the only reactants.

431) The composition of any of embodiments 417-429 wherein the molar ratio of diisocyanate to polyether diol is in the range of 0.95 to 1.05.

432) The composition of any of embodiments 339-385 wherein one or both of the first and second polymer is the reaction product of a diisocyanate and a diamine.

433) The composition of embodiment 432 wherein the diamine is a polyether diamine.

434) The composition of embodiment 433 wherein the polyether diamine comprises at least one type of oxyalkylene sequence selected from the group consisting of oxyethylene, oxypropylene, oxytrimethylene and oxytetramethylene sequences.

435) The composition of any of embodiments 433 or 434 wherein the polyetherdiamine is a blend of polyetherdiamines.

436) The composition of any of embodiments 433 or 434 wherein the polyether diamine is not a blend of polyetherdiamines.

437) The composition of any of embodiments 433-436 wherein the polyetherdiamine is a random copolymer of two or more oxyalkylene sequences.

438) The composition of any of embodiments 433-436 wherein the polyetherdiamine is a block copolymer of two or more oxyalkylene sequences.

439) The composition of any of embodiments 433-436 wherein the polyetherdiamine is an alternating copolymer of two or more oxyalkylene sequences.

440) The composition of any of embodiments 433-436 wherein the polyetherdiamine is an alternating copolymer of two different oxyalklylene repeat units, for example, oxyethylene and oxypropylene.

441) The composition of any of embodiments 432-440 wherein the diisocyanate is an aliphatic diisocyanate and the reactants do not include an aromatic diisocyanate.

442) The composition of any of embodiments 432-440 wherein the diisocyanate is a mixture of aliphatic diisocyanates and the reactants do not include an aromatic diisocyanate.

443) The composition of any of embodiments 432-440 wherein the diisocyanate is an aromatic diisocyanate and the reactants do not include an aliphatic diisocyanate.

444) The composition of any of embodiments 432-440 wherein the diisocyanate is a mixture of aromatic diisocyanates and the reactants do not include an aliphatic diisocyanate.

445) The composition of any of embodiments 432-440 wherein the diisocyanate is a mixture of aromatic diisocyanate and aliphatic diisocyanate.

446) The composition of any of embodiments 432-440 wherein diisocyanate and diamine are the only reactants.

447) The composition of any of embodiments 432-440 wherein the molar ratio of diisocyanate to polyether diamine is in the range of 0.95 to 1.05.

448) The composition of any of embodiments 339-385 wherein one or both of the first and second polymer is the reaction product of a diisocyanate and either (a) a mixture comprising polyether diol and polycarbonate diol or (b) a polyether polycarbonate diol.

449) The composition of embodiment 448 wherein the polyetherdiol comprises at least one type of oxyalkylene sequence selected from the group consisting of oxyethylene, oxypropylene, oxytrimethylene and oxytetramethylene sequences.

450) The composition of any of embodiments 448 or 449 wherein the polyetherdiol is a blend of polyetherdiols.

451) The composition of any of embodiments 448 or 449 wherein the polyether diol is not a blend of polyetherdiols.

452) The composition of any one of embodiments 448-451 wherein the polyetherdiol is a random copolymer of two or more oxyalkylene sequences.

453) The composition of any one of embodiments 448-451 wherein the polyetherdiol is a block copolymer of two or more oxyalkylene sequences.

454) The composition of any of embodiments 448-451 wherein the polyetherdiol is an alternating copolymer of two or more oxyalkylene sequences.

455) The composition of any of embodiments 448-451 wherein the polyetherdiol is an alternating copolymer of two different oxyalklylene repeat units, for example, oxyethylene and oxypropylene.

456) The composition of any one of embodiments 448-455 wherein the polycarbonate diol is poly(hexamethylene carbonate)diol.

457) The composition of any one of embodiments 448-455 wherein the polycarbonate diol is poly(ethylene-carbonate)diol.

458) The composition of any one of embodiments 448-455 wherein the polycarbonate diol is the reaction product of trimethylene carbonate and a diol.

459) The composition of any one of embodiments 448-458 wherein the diisocyanate is an aliphatic diisocyanate and the reactants do not include an aromatic diisocyanate.

460) The composition of any one of embodiments 448-458 wherein the diisocyanate is a mixture of aliphatic diisocyanates and the reactants do not include an aromatic diisocyanate.

461) The composition of any one of embodiments 448-458 wherein the diisocyanate is an aromatic diisocyanate and the reactants do not include an aliphatic diisocyanate.

462) The composition of any one of embodiments 448-458 wherein the diisocyanate is a mixture of aromatic diisocyanates and the reactants do not include an aliphatic diisocyanate.

463) The composition of any one of embodiments 448-458 wherein the diisocyanate is a mixture of aromatic diisocyanate and aliphatic diisocyanate.

464) The composition of any one of embodiments 448-458 which is further chain extended by reaction with a diol.

465) The composition of any of embodiments 339-385 wherein one or both of the first and second polymer is the reaction product of a diamine and a pre-polymer, where the pre-polymer is the reaction product of a diisocyanate and either (a) a mixture comprising polyether diol and polycarbonate diol or (b) a polyether polycarbonate diol.

466) The composition of embodiment 465 wherein the polyetherdiol comprises at least one type of oxyalkylene sequence selected from the group consisting of oxyethylene, oxypropylene, oxytrimethylene and oxytetramethylene sequences.

467) The composition of any of embodiments 464 or 465 wherein the polyetherdiol is a blend of polyetherdiols.

468) The composition of any of embodiments 464 or 465 wherein the polyether diol is not a blend of polyetherdiols.

469) The composition of any one of embodiments 465-468 wherein the polyetherdiol is a random copolymer of two or more oxyalkylene sequences.

470) The composition of any one of embodiments 465-468 wherein the polyetherdiol is a block copolymer of two or more oxyalkylene sequences.

471) The composition of any of embodiments 465-468 wherein the polyetherdiol is an alternating copolymer of two or more oxyalkylene sequences.

472) The composition of any of embodiments 465-468 wherein the polyetherdiol is an alternating copolymer of two different oxyalklylene repeat units, for example, oxyethylene and oxypropylene.

473) The composition of any one of embodiments 465-472 wherein the polycarbonate diol is poly(hexamethylene carbonate)diol.

474) The composition of any one of embodiments 465-472 wherein the polycarbonate diol is poly(ethylene-carbonate)diol.

475) The composition of any one of embodiments 465-472 wherein the polycarbonate diol is the reaction product of trimethylene carbonate and a diol.

476) The composition of any one of embodiments 465-475 wherein the diisocyanate is an aliphatic diisocyanate and the reactants do not include an aromatic diisocyanate.

477) The composition of any one of embodiments 465-475 wherein the diisocyanate is a mixture of aliphatic diisocyanates and the reactants do not include an aromatic diisocyanate.

478) The composition of any one of embodiments 465-475 wherein the diisocyanate is an aromatic diisocyanate and the reactants do not include an aliphatic diisocyanate.

479) The composition of any one of embodiments 465-475 wherein the diisocyanate is a mixture of aromatic diisocyanates and the reactants do not include an aliphatic diisocyanate.

480) The composition of any one of embodiments 465-475 wherein the diisocyanate is a mixture of aromatic diisocyanate and aliphatic diisocyanate.

481) The composition of any one of embodiments 465-480 wherein the diamine is an aliphatic diamine.

482) The composition of any one of embodiments 465-480 wherein the diamine is a polyether diamine.

483) The composition of any one of embodiments 465-480 wherein the diamine is a blend of diamines.

484) The composition of any one of embodiments 465-480 wherein the diamine is a blend of aliphatic diamine and polyether diamine.

485) The composition of any of embodiments 339-385 wherein one or both of the first and second polymer is the reaction product of a diisocyanate and either (a) a mixture comprising polyether diol and polyester diol or (b) a polyether polyester diol.

486) The composition of embodiment 485 wherein the polyether diol comprises at least one type of oxyalkylene sequence selected from the group consisting of oxyethylene, oxypropylene, oxytrimethylene and oxytetramethylene sequences.

487) The composition of any of embodiments 485 or 486 wherein the polyether diol is a blend of polyetherdiols.

488) The composition of any of embodiments 485 or 486 wherein the polyether diol is not a blend of polyetherdiols.

489) The composition of any one of embodiments 485-488 wherein the polyether diol is a random copolymer of two or more oxyalkylene sequences.

490) The composition of any one of embodiments 485-488 wherein the polyether diol is a block copolymer of two or more oxyalkylene sequences.

491) The composition of any of embodiments 485-488 wherein the polyetherdiol is an alternating copolymer of two or more oxyalkylene sequences.

492) The composition of any of embodiments 485-488 wherein the polyetherdiol is an alternating copolymer of two different oxyalklylene repeat units, for example, oxyethylene and oxypropylene.

493) The composition of any one of embodiments 485-492 wherein the diisocyanate is an aliphatic diisocyanate and the reactants do not include an aromatic diisocyanate.

494) The composition of any one of embodiments 485-492 wherein the diisocyanate is a mixture of aliphatic diisocyanates and the reactants do not include an aromatic diisocyanate.

495) The composition of any one of embodiments 485-492 wherein the diisocyanate is an aromatic diisocyanate and the reactants do not include an aliphatic diisocyanate.

496) The composition of any one of embodiments 485-492 wherein the diisocyanate is a mixture of aromatic diisocyanates and the reactants do not include an aliphatic diisocyanate.

497) The composition of any one of embodiments 485-492 wherein the diisocyanate is a mixture of aromatic diisocyanate and aliphatic diisocyanate.

498) The composition of any of embodiments 339-385 wherein one or both of the first and second polymer is the reaction product of a diamine and a pre-polymer, where the pre-polymer is the reaction product of a diisocyanate and either (a) a mixture comprising polyether diol and polyester diol or (b) a polyether polyester diol.

499) The composition of embodiment 498 wherein the polyetherdiol comprises at least one type of oxyalkylene sequence selected from the group consisting of oxyethylene, oxypropylene, oxytrimethylene and oxytetramethylene sequences.

500) The composition of one of embodiments 498 or 499 wherein the polyetherdiol is a blend of polyetherdiols.

501) The composition of any one of embodiments 498 or 499 wherein the polyether diol is not a blend of polyetherdiols.

502) The composition of any one of embodiments 498-501 wherein the polyetherdiol is a random copolymer of two or more oxyalkylene sequences.

503) The composition of any one of embodiments 498-501 wherein the polyetherdiol is a block copolymer of two or more oxyalkylene sequences.

504) The composition of any of embodiments 498-501 wherein the polyetherdiol is an alternating copolymer of two or more oxyalkylene sequences.

505) The composition of any of embodiments 498-501 wherein the polyetherdiol is an alternating copolymer of two different oxyalklylene repeat units, for example, oxyethylene and oxypropylene.

506) The composition of any one of embodiments 498-505 wherein the diisocyanate is an aliphatic diisocyanate and the reactants do not include an aromatic diisocyanate.

507) The composition of any one of embodiments 498-505 wherein the diisocyanate is a mixture of aliphatic diisocyanates and the reactants do not include an aromatic diisocyanate.

508) The composition of any one of embodiments 498-505 wherein the diisocyanate is an aromatic diisocyanate and the reactants do not include an aliphatic diisocyanate.

509) The composition of any one of embodiments 498-505 wherein the diisocyanate is a mixture of aromatic diisocyanates and the reactants do not include an aliphatic diisocyanate.

510) The composition of any one of embodiments 498-505 wherein the diisocyanate is a mixture of aromatic diisocyanate and aliphatic diisocyanate.

511) The composition of any one of embodiments 498-510 wherein the diamine is an aliphatic diamine.

512) The composition of any one of embodiments 498-510 wherein the diamine is a polyether diamine.

513) The composition of any one of embodiments 498-510 wherein the diamine is a blend of diamines.

514) The composition of any one of embodiments 498-510 wherein the diamine is a blend of aliphatic diamine and polyether diamine.

515) The composition any of one of embodiments 339-514 wherein at least one of the first and second polymer is bio-stable.

516) The composition any of one of embodiments 339-514 wherein at least one of the first and second polymers absorbs at least 50% of its weight in water when immersed in 1% aqueous methyl cellulose at 37° C. for 16 hours.

517) The composition any of one of embodiments 339-514 wherein at least one of the first and second polymers has a COF of 0.001 to 0.15.

518) The composition any of one of embodiments 339-514 wherein at least one of the first and second polymers has an intrinsic viscosity of 3-8 dl/g.

519) A method for forming a multilayer polymeric composition, the method comprising

(a) providing a first solution comprising a first polymer;

(b) providing a second solution comprising a second polymer, the first and second polymers being non-identical;

(c) providing a support in the shape of a bone or a portion of a bone;

(d) depositing the first solution onto the support or portion thereof to provide a first coated support;

(e) depositing the first solution onto the first coated support a plurality of times to provide a first layer on the support or portion thereof;

(f) depositing the second solution onto the first layer to provide a second coated support; and

(g) depositing the second solution onto the second coated support a plurality of times to provide a second layer, the multilayer composition comprising the first layer and the second layer.

520) The method of embodiment 519 wherein the support or a coated support is dipped into a first solution or a second solution to provide the depositing.

521) The method of embodiment 520 wherein the first solution has a viscosity of 1 cps to 100 k cps, or 1 cps to 50 k cps, or 1 cps to 25 k cps, or 1 cps to 10 k cps, or 1 cps to 5 k cps, or 1 cps to 2.5 k cps, or 5 cps to 2.5 k cps, or 85 cps to 2 k cps, or 85 cps to 2.5 k cps, or 85 cps to 1.5 k cps, or 85 cps to 1 k cps, or 150 cps to 1 k cps.

522) The method of embodiment 520 wherein the first solution contains the first polymer at a concentration of 1% to 10% per weight, based on weight of solution.

523) The method of embodiment 520 wherein the support or coated support is spun while it is being dipped into the first solution or the second solution.

524) The method of embodiment 520 wherein the support or coated support is held static in the first solution or the second solution during the dipping process.

525) The method of embodiment 520 wherein the first solution is stirred while the support is dipped in the first solution.

526) The method of embodiment 520 wherein the support or coated support is dipped twice in the first solution before allowing solvent to evaporate from the coated support.

527) The method of embodiment 520 wherein the first solution and the second solution contain the same solvent(s).

528) The method of embodiment 520 wherein where the support or coated support is dipped for 1 to 30 seconds in the first solution.

529) The method of embodiment 519 wherein the first solution or second solution is sprayed onto the support or the first coated support.

530) The method of embodiment 529 wherein the support or coated support is spun while it is being sprayed.

531) The method of embodiment 529 wherein the first solution has a viscosity of 1 cps to 100 k cps, or 1 cps to 50 k cps, or 1 cps to 25 k cps, or 1 cps to 10 k cps, or 1 cps to 5 k cps, or 1 cps to 2.5 k cps, or 5 cps to 2.5 k cps, or 85 cps to 2 k cps, or 85 cps to 2.5 k cps, or 85 cps to 1.5 k cps, or 85 cps to 1 k cps, or 150 cps to 1 k cps.

532) The method of 519 further comprising evaporating solvent from a layer of the first solution on the first coated support, prior to deposition of a further layer of the first solution onto the first coated support.

533) The method of embodiment 532 wherein the first coated support is dry to the touch when a further layer of first solution is deposited onto the first coated support.

534) The method of embodiment 532 wherein the first coated support is tacky when a further layer of first solution is deposited onto the first coated support.

535) The method of embodiment 532 wherein the solvent evaporates into an environment having a humidity of 10-85%.

536) The method of embodiment 532 wherein the solvent evaporates into an environment having a pressure of less than atmospheric pressure.

537) The method of embodiment 532 wherein the solvent evaporates into an environment that is warmer than the temperature of the first solution.

538) The method of embodiment 532 wherein the solvent evaporates into an environment that is colder than the temperature of the first solution.

539) The method of embodiment 532 wherein the solvent evaporates for a time of 5 to 120 minutes.

540) The method of embodiment 532 wherein the support or coated support is rotated during solvent evaporation.

541) The method of embodiment 519 wherein a solution is deposited 5 to 100 times onto the support or coated support to form a layer.

542) The method of embodiment 519 wherein the support has spatial dimensions copied from spatial dimensions of a bone of a patient for whom the multilayer polymeric composition is intended.

543) The method of embodiment 519 wherein a solution further comprises a pharmaceutically active agent.

544) The method of embodiment 519 further comprising placing the multilayer composition into a solution that comprises a pharmaceutically active agent, and absorbing pharmaceutically active agent into the multilayer composition.

545) The method of embodiment 519 further comprising separating the multilayer composition from the support.

546) The method of embodiment 545 wherein the multilayer composition is soaked in an aqueous medium before being separated from the support.

547) The method of embodiment 545 wherein the multilayer composition is soaked in a non-aqueous medium before being separated from the support.

548) The method of any of embodiments 519-547 wherein the first polymer further comprises a plurality of urea groups.

549) The method of any of embodiments 519-547 wherein the first polymer further comprises a plurality of ether groups.

550) The method of any of embodiments 519-547 wherein the first polymer further comprises a plurality of carbonate groups.

551) The method of any of embodiments 519-547 wherein the first layer is more hydrophilic than the second layer.

552) The method of any of embodiments 519-547 wherein the first layer is less hydrophilic than the second layer.

553) The method of any of embodiments 519-547 wherein the first layer is more elastic than the second layer.

554) The method of any of embodiments 519-547 wherein the first layer is less elastic than the second layer

555) The method of any of embodiments 519-547 wherein the first layer has a greater hardness than the second layer.

556) The method of any of embodiments 519-547 wherein the first layer has a higher surface modulus than the second layer.

557) The method of any of embodiments 519-547 wherein the first layer has a greater toughness than the second layer.

558) The method of any of embodiments 519-547 wherein the first layer has a higher Tg than the second layer.

559) The method of any of embodiments 519-547 wherein the first layer has a lower Tg than the second layer

560) The method of any of embodiments 519-547 wherein the first layer has a thickness of 0.1 to 1.5 mm.

561) The method of any of embodiments 519-547 wherein the first layer has a thickness of 0.1 to 1.0 mm.

562) The method of any of embodiments 519-547 wherein the first layer has a thickness of 0.1 to 0.5 mm.

563) The method of any of embodiments 519-562 having exactly two layers.

564) The method of embodiment 519 further comprising

i. (h) providing a third solution comprising a third polymer, where the third polymer is non-identical to the second polymer;

ii. depositing the third solution onto the second layer to provide a third coated support;

iii. (j) depositing the third solution onto the third coated support a plurality of times to provide a third layer, the multilayer composition comprising the first layer, the second layer and the third layer.

565) The method of embodiment 564 to provide a multilayer composition having exactly three layers.

566) The method of embodiment 564, the first and third polymers being identical

567) The method of embodiment 564, the first and third polymers being non-identical.

568) The method of embodiment 564, wherein the second layer is more hydrophilic than either the first layer or the third layer.

569) The method of embodiment 564, wherein the second layer is more hydrophilic than the first layer.

570) The method of embodiment 564, wherein the second polymer contains a greater weight percent of ether linkages compared to the first polymer, and wherein the second polymer is more hydrophilic than either the first polymer or the third polymer.

571) The method of embodiment 564, wherein the second polymer contains a greater weight percent of ether linkages compared to the first polymer, and wherein the second polymer is more hydrophilic than the first polymer.

572) The method of embodiment 564, wherein the second layer is more elastic than either the first layer or the third layer.

573) The method of embodiment 564, wherein the second layer is more elastic than the first layer.

574) The method of embodiment 564, wherein the first layer has a greater hardness than the second layer.

575) The method of embodiment 564, wherein the first layer has a higher surface modulus than the second layer.

576) The method of embodiment 564, wherein the first layer has a greater toughness than the second layer.

577) The method of any of embodiments 519-576 wherein the first layer contains a pharmaceutically active agent.

578) The method of any of embodiments 519-576 wherein the second layer contains a pharmaceutically active agent.

579) The method of any of embodiments 519-576 wherein the multilayer composition has a thickness of 1.0 to 3.0 mm.

580) The method of any of embodiments 519-576 wherein the multilayer composition has a thickness of 1.5 to 2.0 mm.

581) The method of any of embodiments 519-576 wherein the multilayer composition is marked.

582) The method of any of embodiments 519-576 wherein the multilayer composition is sterile.

583) The method of any of embodiments 519-576 wherein the multilayer composition has a longest straight line dimension of 1 to 5 cm or is in the form of a cap having a half-circumference of 10-200 mm.

584) The method of any of embodiments 519-583 wherein one or both of the first and second polymer is the reaction product of a pre-polymer and a diamine, where the pre-polymer is the reaction product of a diisocyanate and a polyetherdiol.

585) The method of embodiment 584 wherein the polyetherdiol comprises at least one type of oxyalkylene sequence selected from the group consisting of oxyethylene, oxypropylene, oxytrimethylene and oxytetramethylene sequences.

586) The method of any of embodiments 583 or 584 wherein the polyetherdiol is a blend of polyetherdiols.

587) The method of any of embodiments 583 or 584 wherein the polyether diol is not a blend of polyetherdiols.

588) The method of any of embodiments 519-522 wherein the polyetherdiol is a random copolymer of two or more oxyalkylene sequences.

589) The method of any of embodiments 519-522 wherein the polyetherdiol is a block copolymer of two or more oxyalkylene sequences.

590) The composition of any of embodiments 519-522 wherein the polyetherdiol is an alternating copolymer of two or more oxyalkylene sequences.

591) The composition of any of embodiments 519-522 wherein the polyetherdiol is an alternating copolymer of two different oxyalklylene repeat units, for example, oxyethylene and oxypropylene.

592) The method of any of embodiments 519-524 wherein the diamine is an aliphatic diamine.

593) The method of any of embodiments 519-524 wherein the diamine is a polyether diamine.

594) The method of any of embodiments 519-524 wherein the diamine is a blend of diamines.

595) The method of any of embodiments 519-524 wherein the diamine is a blend of aliphatic diamine and polyether diamine.

596) The method of any of embodiments 519-583 wherein one or both of the first and second polymer is the reaction product of diisocyanate and a polyetherdiamine to form a pre-polymer, and the reaction product of the pre-polymer and a diol to form a polyether urea urethane.

597) The method of embodiment 596 wherein the polyetherdiamine comprises at least one type of oxyalkylene sequence selected from the group consisting of oxyethylene, oxypropylene, oxytrimethylene and oxytetramethylene sequences.

598) The method of any of embodiments 596 or 597 wherein the polyetherdiamine is a blend of polyetherdiamines.

599) The method of any of embodiments 596 or 597 wherein the polyetherdiamine is not a blend of polyetherdiamines.

600) The method of any of embodiments 596-599 wherein the polyetherdiamine is a random copolymer of two or more oxyalkylene sequences.

601) The method of any of embodiments 596-599 wherein the polyetherdiamine is a block copolymer of two or more oxyalkylene sequences.

602) The composition of any of embodiments 596-599 wherein the polyetherdiamine is an alternating copolymer of two or more oxyalkylene sequences.

603) The composition of any of embodiments 596-599 wherein the polyetherdiamine is an alternating copolymer of two different oxyalklylene repeat units, for example, oxyethylene and oxypropylene.

604) The method of any of embodiments 596-603 wherein the diol is an aliphatic diol.

605) The method of any of embodiments 596-603 wherein the diol is an aromatic diol.

606) The method of any of embodiments 596-603 wherein the diol is a polyether diol.

607) The method of any of embodiments 596-603 wherein the diol is a blend of diols.

608) The method of any of embodiments 596-603 wherein the diol is a blend of aliphatic diol and polyetherdiol.

609) The method of any of embodiments 596-608 wherein the diisocyanate is an aliphatic diisocyanate and the reactants do not include an aromatic diisocyanate to form a polyether urea urethane.

610) The method of any of embodiments 596-608 wherein the diisocyanate is a mixture of aliphatic diisocyanates and the reactants do not include an aromatic diisocyanate to form a polyether urea urethane.

611) The method of any of embodiments 596-608 wherein the diisocyanate is an aromatic diisocyanate and the reactants do not include an aliphatic diisocyanate to form a polyether urea urethane.

612) The method of any of embodiments 596-608 wherein the diisocyanate is a mixture of aromatic diisocyanates and the reactants do not include an aliphatic diisocyanate to form a polyether urea urethane.

613) The method of any of embodiments 596-608 wherein the diisocyanate is a mixture of aromatic diisocyanate and aliphatic diisocyanate to form a polyether urea urethane.

614) The method of any of embodiments 519-583 wherein one or both of the first and second polymer is the reaction product of a diisocyanate and a diol.

615) The method of embodiment 614 wherein the diol is a polyether diol.

616) The method of embodiment 615 wherein the polyether diol comprises at least one type of oxyalkylene sequence selected from the group consisting of oxyethylene, oxypropylene, oxytrimethylene and oxytetramethylene sequences.

617) The method of any of embodiments 614 or 615 wherein the polyetherdiol is a blend of polyetherdiols.

618) The method of any of embodiments 614 or 615 wherein the polyether diol is not a blend of polyetherdiols.

619) The method of any of embodiments 614-617 wherein the polyetherdiol is a random copolymer of two or more oxyalkylene sequences.

620) The method of any of embodiments 614-617 wherein the polyetherdiol is a block copolymer of two or more oxyalkylene sequences.

621) The method of any of embodiments 614-617 wherein the polyetherdiol is an alternating copolymer of two or more oxyalkylene sequences.

622) The method of any of embodiments 614-617 wherein the polyetherdiol is an alternating copolymer of two different oxyalklylene repeat units, for example, oxyethylene and oxypropylene.

623) The method of any of embodiments 614-622 wherein the diisocyanate is an aliphatic diisocyanate and the reactants do not include an aromatic diisocyanate.

624) The method of any of embodiments 614-622 wherein the diisocyanate is a mixture of aliphatic diisocyanates and the reactants do not include an aromatic diisocyanate.

625) The method of any of embodiments 614-622 wherein the diisocyanate is an aromatic diisocyanate and the reactants do not include an aliphatic diisocyanate.

626) The method of any of embodiments 614-622 wherein the diisocyanate is a mixture of aromatic diisocyanates and the reactants do not include an aliphatic diisocyanate.

627) The method of any of embodiments 614-622 wherein the diisocyanate is a mixture of aromatic diisocyanate and aliphatic diisocyanate.

628) The method of any of embodiments 614-627 wherein diisocyanate and diol are the only reactants.

629) The method of any of embodiments 614-628 wherein the molar ratio of diisocyanate to polyether diol is in the range of 0.95 to 1.05.

630) The method of any of embodiments 519-583 wherein one or both of the first and second polymer is the reaction product of a diisocyanate and a diamine.

631) The method of embodiment 630 wherein the diamine is a polyether diamine.

632) The method of embodiment 631 wherein the polyether diamine comprises at least one type of oxyalkylene sequence selected from the group consisting of oxyethylene, oxypropylene, oxytrimethylene and oxytetramethylene sequences.

633) The method of any of embodiments 631 or 632 wherein the polyetherdiamine is a blend of polyetherdiamines.

634) The method of any of embodiments 631 or 632 wherein the polyether diamine is not a blend of polyetherdiamines.

635) The method of any of embodiments 631-634 wherein the polyetherdiamine is a random copolymer of two or more oxyalkylene sequences.

636) The method of any of embodiments 631-634 wherein the polyetherdiamine is a block copolymer of two or more oxyalkylene sequences.

637) The method of any of embodiments 631-634 wherein the polyetherdiol is an alternating copolymer of two or more oxyalkylene sequences.

638) The method of any of embodiments 631-634 wherein the polyetherdiol is an alternating copolymer of two different oxyalklylene repeat units, for example, oxyethylene and oxypropylene.

639) The method of any of embodiments 631-638 wherein the diisocyanate is an aliphatic diisocyanate and the reactants do not include an aromatic diisocyanate.

640) The method of any of embodiments 631-638 wherein the diisocyanate is a mixture of aliphatic diisocyanates and the reactants do not include an aromatic diisocyanate.

641) The method of any of embodiments 631-638 wherein the diisocyanate is an aromatic diisocyanate and the reactants do not include an aliphatic diisocyanate.

642) The method of any of embodiments 631-638 wherein the diisocyanate is a mixture of aromatic diisocyanates and the reactants do not include an aliphatic diisocyanate.

643) The method of any of embodiments 631-638 wherein the diisocyanate is a mixture of aromatic diisocyanate and aliphatic diisocyanate.

644) The method of any of embodiments 631-643 wherein diisocyanate and diamine are the only reactants.

645) The method of any of embodiments 631-644 wherein the molar ratio of diisocyanate to polyether diamine is in the range of 0.95 to 1.05.

646) The method of any of embodiments 519-583 wherein one or both of the first and second polymer is the reaction product of a diisocyanate and either (a) a mixture comprising polyether diol and polycarbonate diol or (b) a polyether polycarbonate diol.

647) The method of embodiment 646 wherein the polyetherdiol comprises at least one type of oxyalkylene sequence selected from the group consisting of oxyethylene, oxypropylene, oxytrimethylene and oxytetramethylene sequences.

648) The method of any of embodiments 646 or 647 wherein the polyetherdiol is a blend of polyetherdiols.

649) The method of any of embodiments 646 or 647 wherein the polyether diol is not a blend of polyetherdiols.

650) The method of any one of embodiments 646-649 wherein the polyetherdiol is a random copolymer of two or more oxyalkylene sequences.

651) The method of any one of embodiments 646-649 wherein the polyetherdiol is a block copolymer of two or more oxyalkylene sequences.

652) The method of any of embodiments 646-649 wherein the polyetherdiol is an alternating copolymer of two or more oxyalkylene sequences.

653) The method of any of embodiments 646-649 wherein the polyetherdiol is an alternating copolymer of two different oxyalklylene repeat units, for example, oxyethylene and oxypropylene.

654) The method of any one of embodiments 646-653 wherein the polycarbonate diol is poly(hexamethylene carbonate)diol.

655) The method of any one of embodiments 646-653 wherein the polycarbonate diol is poly(ethylene-carbonate)diol.

656) The method of any one of embodiments 646-653 wherein the polycarbonate diol is the reaction product of trimethylene carbonate and a diol.

657) The method of any one of embodiments 646-656 wherein the diisocyanate is an aliphatic diisocyanate and the reactants do not include an aromatic diisocyanate.

658) The method of any one of embodiments 646-656 wherein the diisocyanate is a mixture of aliphatic diisocyanates and the reactants do not include an aromatic diisocyanate.

659) The method of any one of embodiments 646-656 wherein the diisocyanate is an aromatic diisocyanate and the reactants do not include an aliphatic diisocyanate.

660) The method of any one of embodiments 646-656 wherein the diisocyanate is a mixture of aromatic diisocyanates and the reactants do not include an aliphatic diisocyanate.

661) The method of any one of embodiments 646-656 wherein the diisocyanate is a mixture of aromatic diisocyanate and aliphatic diisocyanate.

662) The method of any one of embodiments 646-661 further comprising chain extending by reaction with a diol.

663) The method of any of embodiments 519-583 wherein one or both of the first and second polymer is the reaction product of a diamine and a pre-polymer, where the pre-polymer is the reaction product of a diisocyanate and either (a) a mixture comprising polyether diol and polycarbonate diol or (b) a polyether polycarbonate diol.

664) The method of embodiment 663 wherein the polyetherdiol comprises at least one type of oxyalkylene sequence selected from the group consisting of oxyethylene, oxypropylene, oxytrimethylene and oxytetramethylene sequences.

665) The method of any of embodiments 663 or 664 wherein the polyetherdiol is a blend of polyetherdiols.

666) The method of any of embodiments 663 or 664 wherein the polyether diol is not a blend of polyetherdiols.

667) The method of any one of embodiments 663-666 wherein the polyetherdiol is a random copolymer of two or more oxyalkylene sequences.

668) The method of any one of embodiments 663-666 wherein the polyetherdiol is a block copolymer of two or more oxyalkylene sequences.

669) The method of any of embodiments 663-666 wherein the polyetherdiol is an alternating copolymer of two or more oxyalkylene sequences.

670) The method of any of embodiments 663-666 wherein the polyetherdiol is an alternating copolymer of two different oxyalklylene repeat units, for example, oxyethylene and oxypropylene.

671) The method of any one of embodiments 663-670 wherein the polycarbonate diol is poly(hexamethylene carbonate)diol.

672) The method of any one of embodiments 663-670 wherein the polycarbonate diol is poly(ethylene-carbonate)diol.

673) The method of any one of embodiments 663-670 wherein the polycarbonate diol is the reaction product of trimethylene carbonate and a diol.

674) The method of any one of embodiments 663-673 wherein the diisocyanate is an aliphatic diisocyanate and the reactants do not include an aromatic diisocyanate.

675) The method of any one of embodiments 663-673 wherein the diisocyanate is a mixture of aliphatic diisocyanates and the reactants do not include an aromatic diisocyanate.

676) The method of any one of embodiments 663-673 wherein the diisocyanate is an aromatic diisocyanate and the reactants do not include an aliphatic diisocyanate.

677) The method of any one of embodiments 663-673 wherein the diisocyanate is a mixture of aromatic diisocyanates and the reactants do not include an aliphatic diisocyanate.

678) The method of any one of embodiments 663-673 wherein the diisocyanate is a mixture of aromatic diisocyanate and aliphatic diisocyanate.

679) The method of any one of embodiments 663-678 wherein the diamine is an aliphatic diamine.

680) The method of any one of embodiments 663-678 wherein the diamine is a polyether diamine.

681) The method of any one of embodiments 663-678 wherein the diamine is a blend of diamines.

682) The method of any one of embodiments 663-678 wherein the diamine is a blend of aliphatic diamine and polyether diamine.

683) The method of any of embodiments 519-583 wherein one or both of the first and second polymer is the reaction product of a diisocyanate and either (a) a mixture comprising polyether diol and polyester diol or (b) a polyether polyester diol.

684) The method of embodiment 683 wherein the polyether diol comprises at least one type of oxyalkylene sequence selected from the group consisting of oxyethylene, oxypropylene, oxytrimethylene and oxytetramethylene sequences.

685) The method of any of embodiments 683 or 684 wherein the polyether diol is a blend of polyetherdiols.

686) The method of any of embodiments 683 or 684 wherein the polyether diol is not a blend of polyetherdiols.

687) The method of any one of embodiments 683-686 wherein the polyether diol is a random copolymer of two or more oxyalkylene sequences.

688) The method of any one of embodiments 683-686 wherein the polyether diol is a block copolymer of two or more oxyalkylene sequences.

689) The method of any of embodiments 683-686 wherein the polyetherdiol is an alternating copolymer of two or more oxyalkylene sequences.

690) The method of any of embodiments 683-686 wherein the polyetherdiol is an alternating copolymer of two different oxyalklylene repeat units, for example, oxyethylene and oxypropylene.

691) The method of any one of embodiments 683-690 wherein the diisocyanate is an aliphatic diisocyanate and the reactants do not include an aromatic diisocyanate.

692) The method of any one of embodiments 683-690 wherein the diisocyanate is a mixture of aliphatic diisocyanates and the reactants do not include an aromatic diisocyanate.

693) The method of any one of embodiments 683-690 wherein the diisocyanate is an aromatic diisocyanate and the reactants do not include an aliphatic diisocyanate.

694) The method of any one of embodiments 683-690 wherein the diisocyanate is a mixture of aromatic diisocyanates and the reactants do not include an aliphatic diisocyanate.

695) The method of any one of embodiments 683-690 wherein the diisocyanate is a mixture of aromatic diisocyanate and aliphatic diisocyanate.

696) The method of any of embodiments 519-583 wherein one or both of the first and second polymer is the reaction product of a diamine and a pre-polymer, where the pre-polymer is the reaction product of a diisocyanate and either (a) a mixture comprising polyether diol and polyester diol or (b) a polyether polyester diol.

697) The method of embodiment 696 wherein the polyetherdiol comprises at least one type of oxyalkylene sequence selected from the group consisting of oxyethylene, oxypropylene, oxytrimethylene and oxytetramethylene sequences.

698) The method of one of embodiments 696 or 697 wherein the polyetherdiol is a blend of polyetherdiols.

699) The method of any one of embodiments 696 or 697 wherein the polyether diol is not a blend of polyetherdiols.

700) The method of any one of embodiments 696-699 wherein the polyetherdiol is a random copolymer of two or more oxyalkylene sequences.

701) The method of any one of embodiments 696-699 wherein the polyetherdiol is a block copolymer of two or more oxyalkylene sequences.

702) The method of any of embodiments 696-699 wherein the polyetherdiol is an alternating copolymer of two or more oxyalkylene sequences.

703) The method of any of embodiments 696-699 wherein the polyetherdiol is an alternating copolymer of two different oxyalklylene repeat units, for example, oxyethylene and oxypropylene.

704) The method of any one of embodiments 696-703 wherein the diisocyanate is an aliphatic diisocyanate and the reactants do not include an aromatic diisocyanate.

705) The method of any one of embodiments 696-703 wherein the diisocyanate is a mixture of aliphatic diisocyanates and the reactants do not include an aromatic diisocyanate.

706) The method of any one of embodiments 696-703 wherein the diisocyanate is an aromatic diisocyanate and the reactants do not include an aliphatic diisocyanate.

707) The method of any one of embodiments 696-703 wherein the diisocyanate is a mixture of aromatic diisocyanates and the reactants do not include an aliphatic diisocyanate.

708) The method of any one of embodiments 696-703 wherein the diisocyanate is a mixture of aromatic diisocyanate and aliphatic diisocyanate.

709) The method of any one of embodiments 696-708 wherein the diamine is an aliphatic diamine.

710) The method of any one of embodiments 696-708 wherein the diamine is a polyether diamine.

711) The method of any one of embodiments 696-708 wherein the diamine is a blend of diamines.

712) The method of any one of embodiments 696-708 wherein the diamine is a blend of aliphatic diamine and polyether diamine.

713) The method any of one of embodiments 519-712 wherein at least one of the first and second polymers is bio-stable.

714) The method any of one of embodiments 519-712 wherein at least one of the first and second polymers absorbs at least 50% of its weight in water when immersed in 1% aqueous methyl cellulose at 37° C. for 16 hours.

715) The method any of one of embodiments 519-712 wherein at least one of the first and second polymers has a COF of 0.001 to 0.15.

716) The method any of one of embodiments 519-712 wherein at least one of the first and second polymers has an intrinsic viscosity of 3-8 dl/g.

717) A method for forming a multilayer polymeric composition, the method comprising

(a) providing a first solution comprising a first polymer;

(b) providing a second solution comprising a second polymer, the first and second polymers being non-identical;

(c) providing a support in the shape of a bone or a portion of a bone;

(d) depositing the first solution onto a portion of the support to provide a first coated support;

(e) depositing the first solution onto the first coated support a plurality of times to provide a first layer on the portion of the support;

(f) removing the first layer from the support;

(g) inverting the first layer to provide an inverted first layer, and placing the inverted first layer onto the support to provide an inverted first support coating;

(h) depositing the second solution onto the inverted first support coating to provide a second coated support; and

(i) depositing the second solution onto the second coated support a plurality of times to provide a second layer, the multilayer composition comprising the first layer and the second layer.

718) The method of embodiment 717 wherein the support or a coated support is dipped into a first solution or a second solution to provide the depositing.

719) The method of embodiment 718 wherein the first solution has a viscosity of 1 cps to 100 k cps, or 1 cps to 50 k cps, or 1 cps to 25 k cps, or 1 cps to 10 k cps, or 1 cps to 5 k cps, or 1 cps to 2.5 k cps, or 5 cps to 2.5 k cps, or 85 cps to 2 k cps, or 85 cps to 2.5 k cps, or 85 cps to 1.5 k cps, or 85 cps to 1 k cps, or 150 cps to 1 k cps.

720) The method of embodiment 718 wherein the first solution contains the first polymer at a concentration of 1% to 10% per weight, based on weight of solution.

721) The method of embodiment 718 wherein the support or coated support is spun while it is being dipped into the first solution or the second solution.

722) The method of embodiment 718 wherein the support or coated support is held static in the first solution or the second solution during the dipping process.

723) The method of embodiment 718 wherein the first solution is stirred while the support is dipped in the first solution.

724) The method of embodiment 718 wherein the support or coated support is dipped twice in the first solution before allowing solvent to evaporate from the coated support.

725) The method of embodiment 718 wherein the first solution and the second solution contain the same solvent(s).

726) The method of embodiment 718 wherein where the support or coated support is dipped for 1 to 30 seconds in the first solution.

727) The method of embodiment 717 wherein the first solution or second solution is sprayed onto the support or the first coated support.

728) The method of embodiment 727 wherein the support or coated support is spun while it is being sprayed.

729) The method of embodiment 727 wherein the first solution has a viscosity of 1 cps to 100 k cps, or 1 cps to 50 k cps, or 1 cps to 25 k cps, or 1 cps to 10 k cps, or 1 cps to 5 k cps, or 1 cps to 2.5 k cps, or 5 cps to 2.5 k cps, or 85 cps to 2 k cps, or 85 cps to 2.5 k cps, or 85 cps to 1.5 k cps, or 85 cps to 1 k cps, or 150 cps to 1 k cps.

730) The method of 717 further comprising evaporating solvent from a layer of the first solution on the first coated support, prior to deposition of a further layer of the first solution onto the first coated support.

731) The method of embodiment 730 wherein the first coated support is dry to the touch when a further layer of first solution is deposited onto the first coated support.

732) The method of embodiment 730 wherein the first coated support is tacky when a further layer of first solution is deposited onto the first coated support.

733) The method of embodiment 730 wherein the solvent evaporates into an environment having a humidity of 10-85%.

734) The method of embodiment 730 wherein the solvent evaporates into an environment having a pressure of less than atmospheric pressure.

735) The method of embodiment 730 wherein the solvent evaporates into an environment that is warmer than the temperature of the first solution.

736) The method of embodiment 730 wherein the solvent evaporates for a time of 5 to 120 minutes.

737) The method of embodiment 730 wherein the support or coated support is rotated during solvent evaporation.

738) The method of embodiment 717 wherein a solution is deposited 5 to 100 times onto the support or coated support to form a layer.

739) The method of embodiment 717 wherein the support has spatial dimensions copied from spatial dimensions of a bone of a patient for whom the multilayer polymeric composition is intended.

740) The method of embodiment 717 wherein a solution further comprises a pharmaceutically active agent.

741) The method of embodiment 717 further comprising placing the multilayer composition into a solution that comprises a pharmaceutically active agent, and absorbing pharmaceutically active agent into the multilayer composition.

742) The method of embodiment 717 further comprising separating the multilayer composition from the support.

743) The method of embodiments 717-742 wherein the first layer and/or the multilayer composition is soaked in an aqueous medium before being separated from the support.

744) The method of embodiment 717-742 wherein the first layer and/or the multilayer composition is soaked in a non-aqueous medium before being separated from the support.

745) The method of any of embodiments 717-744 wherein the first polymer further comprises a plurality of urea groups.

746) The method of any of embodiments 717-744 wherein the first polymer further comprises a plurality of ether groups.

747) The method of any of embodiments 717-744 wherein the first polymer further comprises a plurality of carbonate groups.

748) The method of any of embodiments 717-744 wherein the first layer is more hydrophilic than the second layer.

749) The method of any of embodiments 717-744 wherein the first layer is less hydrophilic than the second layer.

750) The method of any of embodiments 717-744 wherein the first layer is more elastic than the second layer.

751) The method of any of embodiments 717-744 wherein the first layer is less elastic than the second layer.

752) The method of any of embodiments 717-744 wherein the first layer has a greater hardness than the second layer.

753) The method of any of embodiments 717-744 wherein the first layer has a higher surface modulus than the second layer.

754) The method of any of embodiments 717-744 wherein the first layer has a greater toughness than the second layer.

755) The method of any of embodiments 717-744 wherein the first layer has a higher Tg than the second layer.

756) The method of any of embodiments 717-744 wherein the first layer has a thickness of 0.1 to 1.5 mm.

757) The method of any of embodiments 717-744 wherein the first layer has a thickness of 0.1 to 1.0 mm.

758) The method of any of embodiments 717-744 wherein the first layer has a thickness of 0.1 to 0.5 mm.

759) The method of any of embodiments 717-758 having exactly two layers.

760) The method of embodiment 717 further comprising
(h) providing a third solution comprising a third polymer, where the third polymer is non-identical to the second polymer;
(i) depositing the third solution onto the second layer to provide a third coated support;
(j) depositing the third solution onto the third coated support a plurality of times to provide a third layer, the multilayer composition comprising the first layer, the second layer and the third layer.

761) The method of embodiment 760 to provide a multilayer composition having exactly three layers.

762) The method of embodiment 760, the first and third polymers being identical

763) The method of embodiment 760, the first and third polymers being non-identical.

764) The method of embodiment 760, wherein the second layer is more hydrophilic than either the first layer or the third layer, or alternatively, the second layer is more hydrophilic than the first layer.

765) The method of embodiment 760, wherein the second polymer contains a greater weight percent of ether linkages compared to the first polymer, and wherein the second polymer is more hydrophilic than either the first polymer or the third polymer.

766) The method of embodiment 760, wherein the second polymer contains a greater weight percent of ether linkages compared to the first polymer, and wherein the second polymer is more hydrophilic than the first polymer.

767) The method of embodiment 760, wherein the second layer is more elastic than either the first layer or the third layer.

768) The method of embodiment 760, wherein the second layer is more elastic than the first layer.

769) The method of embodiment 760, wherein the first layer has a greater hardness than the second layer.

770) The method of embodiment 760, wherein the first layer has a higher surface modulus than the second layer.

771) The method of embodiment 760, wherein the first layer has a greater toughness than the second layer.

772) The method of any of embodiments 717-771 wherein the first layer contains a pharmaceutically active agent.

773) The method of any of embodiments 717-771 wherein the second layer contains a pharmaceutically active agent.

774) The method of any of embodiments 717-771 wherein the cap has a thickness of 1.0 to 3.0 mm.

775) The method of any of embodiments 717-771 wherein the cap has a thickness of 1.5 to 2.0 mm.

776) The method of any of embodiments 717-771 wherein the cap is marked.

777) The method of any of embodiments 717-771 wherein the cap is sterile.

778) The method of any of embodiments 717-771 wherein the multilayer composition has a longest straight line dimension of 1 to 5 cm or is in the form of a cap having a half-circumference of 10-200 mm.

779) The method of any of embodiments 717-778 wherein one or both of the first and second polymer is the reaction product of a pre-polymer and a diamine, where the pre-polymer is the reaction product of a diisocyanate and a polyetherdiol.

780) The method of embodiment 779 wherein the polyetherdiol comprises at least one type of oxyalkylene sequence selected from the group consisting of oxyethylene, oxypropylene, oxytrimethylene and oxytetramethylene sequences.

781) The method of any of embodiments 779 or 780 wherein the polyetherdiol is a blend of polyetherdiols.

782) The method of any of embodiments 779 or 780 wherein the polyether diol is not a blend of polyetherdiols.

783) The method of any of embodiments 779-782 wherein the polyetherdiol is a random copolymer of two or more oxyalkylene sequences.

784) The method of any of embodiments 779-782 wherein the polyetherdiol is a block copolymer of two or more oxyalkylene sequences.

785) The method of any of embodiments 779-782 wherein the polyetherdiol is an alternating copolymer of two or more oxyalkylene sequences.

786) The method of any of embodiments 779-782 wherein the polyetherdiol is an alternating copolymer of two different oxyalklylene repeat units, for example, oxyethylene and oxypropylene.

787) The method of any of embodiments 779-786 wherein the diamine is an aliphatic diamine.

788) The method of any of embodiments 779-786 wherein the diamine is a polyether diamine.

789) The method of any of embodiments 779-786 wherein the diamine is a blend of diamines.

790) The method of any of embodiments 779-786 wherein the diamine is a blend of aliphatic diamine and polyether diamine.

791) The method of any of embodiments 717-778 wherein one or both of the first and second polymer is the reaction product of diisocyanate and a polyetherdiamine to form a pre-polymer, and the reaction product of the pre-polymer and a diol to form a polyether urea urethane.

792) The method of embodiment 791 wherein the polyetherdiamine comprises at least one type of oxyalkylene sequence selected from the group consisting of oxyethylene, oxypropylene, oxytrimethylene and oxytetramethylene sequences.

793) The method of any of embodiments 791 or 792 wherein the polyetherdiamine is a blend of polyetherdiamines.

794) The method of any of embodiments 791 or 792 wherein the polyetherdiamine is not a blend of polyetherdiamines.

795) The method of any of embodiments 791-794 wherein the polyetherdiamine is a random copolymer of two or more oxyalkylene sequences.

796) The method of any of embodiments 791-794 wherein the polyetherdiamine is a block copolymer of two or more oxyalkylene sequences.

797) The method of any of embodiments 791-794 wherein the polyetherdiol is an alternating copolymer of two or more oxyalkylene sequences.

798) The method of any of embodiments 791-794 wherein the polyetherdiol is an alternating copolymer of two different oxyalklylene repeat units, for example, oxyethylene and oxypropylene.

799) The method of any of embodiments 791-798 wherein the diol is an aliphatic diol.

800) The method of any of embodiments 791-798 wherein the diol is an aromatic diol.

801) The method of any of embodiments 791-798 wherein the diol is a polyether diol.

802) The method of any of embodiments 791-798 wherein the diol is a blend of diols.

803) The method of any of embodiments 791-798 wherein the diol is a blend of aliphatic diol and polyetherdiol.

804) The method of any of embodiments 791-803 wherein the diisocyanate is an aliphatic diisocyanate and the reactants do not include an aromatic diisocyanate to form a polyether urea urethane.

805) The method of any of embodiments 791-803 wherein the diisocyanate is a mixture of aliphatic diisocyanates and the reactants do not include an aromatic diisocyanate to form a polyether urea urethane.

806) The method of any of embodiments 791-803 wherein the diisocyanate is an aromatic diisocyanate and the reactants do not include an aliphatic diisocyanate to form a polyether urea urethane.

807) The method of any of embodiments 791-803 wherein the diisocyanate is a mixture of aromatic diisocyanates and the reactants do not include an aliphatic diisocyanate to form a polyether urea urethane.

808) The method of any of embodiments 791-803 wherein the diisocyanate is a mixture of aromatic diisocyanate and aliphatic diisocyanate to form a polyether urea urethane.

809) The method of any of embodiments 717-778 wherein one or both of the first and second polymer is the reaction product of a diisocyanate and a diol.

810) The method of embodiment 809 wherein the diol is a polyether diol.

811) The method of embodiment 810 wherein the polyether diol comprises at least one type of oxyalkylene sequence selected from the group consisting of oxyethylene, oxypropylene, oxytrimethylene and oxytetramethylene sequences.

812) The method of any of embodiments 810 or 811 wherein the polyetherdiol is a blend of polyetherdiols.

813) The method of any of embodiments 810 or 811 wherein the polyether diol is not a blend of polyetherdiols.

814) The method of any of embodiments 810-813 wherein the polyetherdiol is a random copolymer of two or more oxyalkylene sequences.

815) The method of any of embodiments 810-813 wherein the polyetherdiol is a block copolymer of two or more oxyalkylene sequences.

816) The composition of any of embodiments 810-813 wherein the polyetherdiol is an alternating copolymer of two or more oxyalkylene sequences.

817) The method of any of embodiments 810-813 wherein the polyetherdiol is an alternating copolymer of two different oxyalklylene repeat units, for example, oxyethylene and oxypropylene.

818) The method of any of embodiments 809-817 wherein the diisocyanate is an aliphatic diisocyanate and the reactants do not include an aromatic diisocyanate.

819) The method of any of embodiments 809-817 wherein the diisocyanate is a mixture of aliphatic diisocyanates and the reactants do not include an aromatic diisocyanate.

820) The method of any of embodiments 809-817 wherein the diisocyanate is an aromatic diisocyanate and the reactants do not include an aliphatic diisocyanate.

821) The method of any of embodiments 809-817 wherein the diisocyanate is a mixture of aromatic diisocyanates and the reactants do not include an aliphatic diisocyanate.

822) The method of any of embodiments 809-817 wherein the diisocyanate is a mixture of aromatic diisocyanate and aliphatic diisocyanate.

823) The method of any of embodiments 809-822 wherein diisocyanate and diol are the only reactants.

824) The method of any of embodiments 809-823 wherein the molar ratio of diisocyanate to polyether diol is in the range of 0.95 to 1.05.

825) The method of any of embodiments 717-778 wherein one or both of the first and second polymer is the reaction product of a diisocyanate and a diamine.

826) The method of embodiment 825 wherein the diamine is a polyether diamine.

827) The method of embodiment 826 wherein the polyether diamine comprises at least one type of oxyalkylene sequence selected from the group consisting of oxyethylene, oxypropylene, oxytrimethylene and oxytetramethylene sequences.

828) The method of any of embodiments 826 or 827 wherein the polyetherdiamine is a blend of polyetherdiamines.

829) The method of any of embodiments 826 or 827 wherein the polyether diamine is not a blend of polyetherdiamines.

830) The method of any of embodiments 826-829 wherein the polyetherdiamine is a random copolymer of two or more oxyalkylene sequences.

831) The method of any of embodiments 826-829 wherein the polyetherdiamine is a block copolymer of two or more oxyalkylene sequences.

832) The method of any of embodiments 826-829 wherein the polyetherdiamine is an alternating copolymer of two or more oxyalkylene sequences.

833) The method of any of embodiments 826-829 wherein the polyetherdiamine is an alternating copolymer of two different oxyalklylene repeat units, for example, oxyethylene and oxypropylene.

834) The method of any of embodiments 826-833 wherein the diisocyanate is an aliphatic diisocyanate and the reactants do not include an aromatic diisocyanate.

835) The method of any of embodiments 826-833 wherein the diisocyanate is a mixture of aliphatic diisocyanates and the reactants do not include an aromatic diisocyanate.

836) The method of any of embodiments 826-833 wherein the diisocyanate is an aromatic diisocyanate and the reactants do not include an aliphatic diisocyanate.

837) The method of any of embodiments 826-833 wherein the diisocyanate is a mixture of aromatic diisocyanates and the reactants do not include an aliphatic diisocyanate.

838) The method of any of embodiments 826-833 wherein the diisocyanate is a mixture of aromatic diisocyanate and aliphatic diisocyanate.

839) The method of any of embodiments 809-838 wherein diisocyanate and diamine are the only reactants.

840) The method of any of embodiments 809-839 wherein the molar ratio of diisocyanate to polyether diamine is in the range of 0.95 to 1.05.

841) The method of any of embodiments 717-778 wherein one or both of the first and second polymer is the reaction product of a diisocyanate and either (a) a mixture comprising polyether diol and polycarbonate diol or (b) a polyether polycarbonate diol.

842) The method of embodiment 841 wherein the polyetherdiol comprises at least one type of oxyalkylene sequence selected from the group consisting of oxyethylene, oxypropylene, oxytrimethylene and oxytetramethylene sequences.

843) The method of any of embodiments 841 or 842 wherein the polyetherdiol is a blend of polyetherdiols.

844) The method of any of embodiments 841 or 842 wherein the polyether diol is not a blend of polyetherdiols.

845) The method of any one of embodiments 841-844 wherein the polyetherdiol is a random copolymer of two or more oxyalkylene sequences.

846) The method of any one of embodiments 841-844 wherein the polyetherdiol is a block copolymer of two or more oxyalkylene sequences.

847) The method of any of embodiments 841-844 wherein the polyetherdiol is an alternating copolymer of two or more oxyalkylene sequences.

848) The method of any of embodiments 841-844 wherein the polyetherdiol is an alternating copolymer of two different oxyalklylene repeat units, for example, oxyethylene and oxypropylene.

849) The method of any one of embodiments 841-848 wherein the polycarbonate diol is poly(hexamethylene carbonate)diol.

850) The method of any one of embodiments 841-848 wherein the polycarbonate diol is poly(ethylene-carbonate)diol.

851) The method of any one of embodiments 841-848 wherein the polycarbonate diol is the reaction product of trimethylene carbonate and a diol.

852) The method of any one of embodiments 841-851 wherein the diisocyanate is an aliphatic diisocyanate and the reactants do not include an aromatic diisocyanate.

853) The method of any one of embodiments 841-851 wherein the diisocyanate is a mixture of aliphatic diisocyanates and the reactants do not include an aromatic diisocyanate.

854) The method of any one of embodiments 841-851 wherein the diisocyanate is an aromatic diisocyanate and the reactants do not include an aliphatic diisocyanate.

855) The method of any one of embodiments 841-851 wherein the diisocyanate is a mixture of aromatic diisocyanates and the reactants do not include an aliphatic diisocyanate.

856) The method of any one of embodiments 841-851 wherein the diisocyanate is a mixture of aromatic diisocyanate and aliphatic diisocyanate.

857) The method of any one of embodiments 841-851 further comprising chain extending by reaction with a diol.

858) The method of any of embodiments 717-778 wherein one or both of the first and second polymer is the reaction product of a diamine and a pre-polymer, where the pre-polymer is the reaction product of a diisocyanate and either (a) a mixture comprising polyether diol and polycarbonate diol or (b) a polyether polycarbonate diol.

859) The method of embodiment 858 wherein the polyetherdiol comprises at least one type of oxyalkylene sequence selected from the group consisting of oxyethylene, oxypropylene, oxytrimethylene and oxytetramethylene sequences.

860) The method of any of embodiments 858 or 859 wherein the polyetherdiol is a blend of polyetherdiols.

861) The method of any of embodiments 858 or 859 wherein the polyether diol is not a blend of polyetherdiols.

862) The method of any one of embodiments 858-861 wherein the polyetherdiol is a random copolymer of two or more oxyalkylene sequences.

863) The method of any one of embodiments 858-861 wherein the polyetherdiol is a block copolymer of two or more oxyalkylene sequences.

864) The method of any of embodiments 858-861 wherein the polyetherdiol is an alternating copolymer of two or more oxyalkylene sequences.

865) The method of any of embodiments 858-861 wherein the polyetherdiol is an alternating copolymer of two different oxyalklylene repeat units, for example, oxyethylene and oxypropylene.

866) The method of any one of embodiments 858-865 wherein the polycarbonate diol is poly(hexamethylene carbonate)diol.

867) The method of any one of embodiments 858-865 wherein the polycarbonate diol is poly(ethylene-carbonate)diol.

868) The method of any one of embodiments 858-865 wherein the polycarbonate diol is the reaction product of trimethylene carbonate and a diol.

869) The method of any one of embodiments 858-868 wherein the diisocyanate is an aliphatic diisocyanate and the reactants do not include an aromatic diisocyanate.

870) The method of any one of embodiments 858-868 wherein the diisocyanate is a mixture of aliphatic diisocyanates and the reactants do not include an aromatic diisocyanate.

871) The method of any one of embodiments 858-868 wherein the diisocyanate is an aromatic diisocyanate and the reactants do not include an aliphatic diisocyanate.

872) The method of any one of embodiments 858-868 wherein the diisocyanate is a mixture of aromatic diisocyanates and the reactants do not include an aliphatic diisocyanate.

873) The method of any one of embodiments 858-868 wherein the diisocyanate is a mixture of aromatic diisocyanate and aliphatic diisocyanate.

874) The method of any one of embodiments 858-873 wherein the diamine is an aliphatic diamine.

875) The method of any one of embodiments 858-873 wherein the diamine is a polyether diamine.

876) The method of any one of embodiments 858-873 wherein the diamine is a blend of diamines.

877) The method of any one of embodiments 858-873 wherein the diamine is a blend of aliphatic diamine and polyether diamine.

878) The method of any of embodiments 717-778 wherein one or both of the first and second polymer is the reaction product of a diisocyanate and either (a) a mixture comprising polyether diol and polyester diol or (b) a polyether polyester diol.

879) The method of embodiment 878 wherein the polyether diol comprises at least one type of oxyalkylene sequence selected from the group consisting of oxyethylene, oxypropylene, oxytrimethylene and oxytetramethylene sequences.

880) The method of any of embodiments 878 or 879 wherein the polyether diol is a blend of polyetherdiols.

881) The method of any of embodiments 878 or 879 wherein the polyether diol is not a blend of polyetherdiols.

882) The method of any one of embodiments 878-881 wherein the polyether diol is a random copolymer of two or more oxyalkylene sequences.

883) The method of any one of embodiments 878-881 wherein the polyether diol is a block copolymer of two or more oxyalkylene sequences.

884) The method of any of embodiments 878-881 wherein the polyetherdiol is an alternating copolymer of two or more oxyalkylene sequences.

885) The method of any of embodiments 878-881 wherein the polyetherdiol is an alternating copolymer of two different oxyalklylene repeat units, for example, oxyethylene and oxypropylene.

886) The method of any one of embodiments 878-885 wherein the diisocyanate is an aliphatic diisocyanate and the reactants do not include an aromatic diisocyanate.

887) The method of any one of embodiments 878-885 wherein the diisocyanate is a mixture of aliphatic diisocyanates and the reactants do not include an aromatic diisocyanate.

888) The method of any one of embodiments 878-885 wherein the diisocyanate is an aromatic diisocyanate and the reactants do not include an aliphatic diisocyanate.

889) The method of any one of embodiments 878-885 wherein the diisocyanate is a mixture of aromatic diisocyanates and the reactants do not include an aliphatic diisocyanate.

890) The method of any one of embodiments 878-885 wherein the diisocyanate is a mixture of aromatic diisocyanate and aliphatic diisocyanate.

891) The method of any of embodiments 717-778 wherein one or both of the first and second polymer is the reaction product of a diamine and a pre-polymer, where the pre-polymer is the reaction product of a diisocyanate and either (a) a mixture comprising polyether diol and polyester diol or (b) a polyether polyester diol.

892) The method of embodiment 891 wherein the polyetherdiol comprises at least one type of oxyalkylene sequence selected from the group consisting of oxyethylene, oxypropylene, oxytrimethylene and oxytetramethylene sequences.

893) The method of one of embodiments 891 or 892 wherein the polyetherdiol is a blend of polyetherdiols.

894) The method of any one of embodiments 891 or 892 wherein the polyether diol is not a blend of polyetherdiols.

895) The method of any one of embodiments 891-894 wherein the polyetherdiol is a random copolymer of two or more oxyalkylene sequences.

896) The method of any one of embodiments 891-894 wherein the polyetherdiol is a block copolymer of two or more oxyalkylene sequences.

897) The method of any of embodiments 891-894 wherein the polyetherdiol is an alternating copolymer of two or more oxyalkylene sequences.

898) The method of any of embodiments 891-894 wherein the polyetherdiol is an alternating copolymer of two different oxyalklylene repeat units, for example, oxyethylene and oxypropylene.

899) The method of any one of embodiments 891-898 wherein the diisocyanate is an aliphatic diisocyanate and the reactants do not include an aromatic diisocyanate.

900) The method of any one of embodiments 891-898 wherein the diisocyanate is a mixture of aliphatic diisocyanates and the reactants do not include an aromatic diisocyanate.

901) The method of any one of embodiments 891-898 wherein the diisocyanate is an aromatic diisocyanate and the reactants do not include an aliphatic diisocyanate.

902) The method of any one of embodiments 891-898 wherein the diisocyanate is a mixture of aromatic diisocyanates and the reactants do not include an aliphatic diisocyanate.

903) The method of any one of embodiments 891-898 wherein the diisocyanate is a mixture of aromatic diisocyanate and aliphatic diisocyanate.

904) The method of any one of embodiments 891-903 wherein the diamine is an aliphatic diamine.

905) The method of any one of embodiments 891-903 wherein the diamine is a polyether diamine.

906) The method of any one of embodiments 891-903 wherein the diamine is a blend of diamines.

907) The method of any one of embodiments 891-903 wherein the diamine is a blend of aliphatic diamine and polyether diamine.

908) The method any of one of embodiments 717-907 wherein at least one of the first and second polymer is bio-stable.

909) The method any of one of embodiments 717-907 wherein at least one of the first and second polymers absorbs at least 50% of its weight in water when immersed in 1% aqueous methyl cellulose at 37° C. for 16 hours.

910) The method any of one of embodiments 717-907 wherein at least one of the first and second polymers has a COF of 0.001 to 0.15.

911) The method any of one of embodiments 717-907 wherein at least one of the first and second polymers has an intrinsic viscosity of 3-8 dl/g.

912) A method for forming a multilayer polymeric composition, the method comprising

(a) providing a first solution comprising a first polymer;

(b) providing a second solution comprising a pre-polymer of a second polymer, the first and second polymers being non-identical;

(c) providing a third solution comprising a reactant reactive with the pre-polymer, the reactant and the pre-polymer being reactive to form the second polymer;

(d) providing a fourth solution comprising a third polymer;

(e) providing a support in the shape of a bone or a portion thereof;

(f) depositing the first solution onto the support or portion thereof to provide a first coated support;

(g) depositing the first solution onto the first coated support a plurality of times to provide a first layer;

(h) depositing the second solution on the first layer to provide a second coated support;

(i) depositing the third solution on the second coated support to form an intermediate layer and provide a third coated support;

(j) depositing the fourth solution onto the third coated support to provide a fourth coated support; and

(k) depositing the fourth solution on the fourth coated support a plurality of times to provide a second layer, the multilayer composition comprising the first layer, the intermediate layer and the second layer.

913) The method of embodiment 912 wherein the prepolymer comprises urethane groups and isocyanate end groups.

914) The method of embodiment 913 wherein the reactant comprises amine end groups.

915) The method of embodiment 913 wherein the reactant is a pre-polymer comprising urethane groups, carbonate groups, and amine end groups.

916) The method of embodiment 912 wherein the prepolymer comprises urethane groups, carbonate groups, and amine end groups.

917) The method of embodiment 916 wherein the reactant comprises isocyanate groups.

918) The method of embodiment 916 wherein the reactant is a pre-polymer comprising urethane groups and isocyanate end groups.

919) The method of embodiment 912-918 wherein the support or a coated support is dipped into a solution to provide the depositing.

920) The method of embodiment 919 wherein a solution has a viscosity of 1 1 cps to 100 k cps, or 1 cps to 50 k cps, or 1 cps to 25 k cps, or 1 cps to 10 k cps, or 1 cps to 5 k cps, or 1 cps to 2.5 k cps, or 5 cps to 2.5 k cps, or 85 cps to 2 k cps, or 85 cps to 2.5 k cps, or 85 cps to 1.5 k cps, or 85 cps to 1 k cps, or 150 cps to 1 k cps.

921) The method of embodiment 919 wherein the first solution contains the first polymer at a concentration of 1% to 10% per weight, based on weight of solution.

922) The method of embodiment 919 wherein the support or coated support is spun while it is being dipped into a solution.

923) The method of embodiment 919 wherein the support or coated support is held static in a solution during the dipping process.

924) The method of embodiment 919 wherein a solution is stirred while the support is dipped in the solution.

925) The method of embodiment 919 wherein the support or coated support is dipped twice in a solution before allowing solvent to evaporate from the coated support.

926) The method of embodiment 919 wherein the first solution and the second solution contain the same solvent(s).

927) The method of embodiment 919 wherein where the support or coated support is dipped for 1 to 30 seconds a solution.

928) The method of embodiment 912-918 wherein a solution is sprayed onto the support or the coated support.

929) The method of embodiment 928 wherein the support or coated support is spun while it is being sprayed.

930) The method of embodiment 928 wherein a solution has a viscosity of 1 cps to 100 k cps, or 1 cps to 50 k cps, or 1 cps to 25 k cps, or 1 cps to 10 k cps, or 1 cps to 5 k cps, or 1 cps to 2.5 k cps, or 5 cps to 2.5 k cps, or 85 cps to 2 k cps, or 85 cps to 2.5 k cps, or 85 cps to 1.5 k cps, or 85 cps to 1 k cps, or 150 cps to 1 k cps.

931) The method of 912-918 further comprising evaporating solvent from the first solution on the first coated support, prior to deposition of further first solution onto the first coated support.

932) The method of embodiment 931 wherein the first coated support is dry to the touch when further first solution is deposited onto the first coated support.

933) The method of embodiment 931 wherein the first coated support is tacky when further first solution is deposited onto the first coated support.

934) The method of embodiment 931 wherein the solvent evaporates into an environment having a humidity of 10-85%.

935) The method of embodiment 931 wherein the solvent evaporates into an environment having a pressure of less than atmospheric pressure.

936) The method of embodiment 931 wherein the solvent evaporates into an environment that is warmer than the temperature of the first solution.

937) The method of embodiment 931 wherein the solvent evaporates into an environment that is cooler than the temperature of the first solution.

938) The method of embodiment 931 wherein the solvent evaporates for a time of 5 to 120 minutes.

939) The method of embodiment 931 wherein the support or coated support is rotated during solvent evaporation.

940) The method of embodiment 912 wherein a solution is deposited 5 to 100 times onto the support or coated support to form a layer.

941) The method of embodiment 912 wherein the support has spatial dimensions copied from spatial dimensions of a bone of a patient for whom the multilayer polymeric composition is intended.

942) The method of embodiment 912 wherein a solution further comprises a pharmaceutically active agent.

943) The method of embodiment 912 further comprising placing the multilayer composition into a solution that comprises a pharmaceutically active agent, and absorbing pharmaceutically active agent into the multilayer composition.

944) The method of embodiment 912 further comprising separating the multilayer composition from the support.

945) The method of embodiment 944 wherein the multilayer composition is soaked in an aqueous medium before being separated from the support.

946) The method of embodiment 944 wherein the multilayer composition is soaked in a non-aqueous medium before being separated from the support.

947) The method of any of embodiments 811-844 wherein each of the first polymer, the second polymer and the third polymer comprises a plurality of urethane groups.

948) The method of embodiment 845 wherein at least one of the first polymer, the second polymer and the third polymer further comprises a plurality of ether groups.

949) The method of embodiment 845 wherein each of the first polymer, the second polymer and the third polymer comprise a plurality of ether groups.

950) The method of embodiment 845 wherein at least one of the first polymer, the second polymer and the third polymer further comprises a plurality of carbonate groups and/or a plurality of urea groups.

951) The method of any of embodiments 912-950 wherein the first layer is more hydrophilic than the second layer.

952) The method of any of embodiments 912-950 wherein the first layer is less hydrophilic than the second layer

953) The method of any of embodiments 912-950 wherein the first layer is more elastic than the second layer.

954) The method of any of embodiments 912-950 wherein the first layer is less elastic than the second layer

955) The method of any of embodiments 912-950 wherein the first layer has a greater hardness than the second layer.

956) The method of any of embodiments 912-950 wherein the first layer has a higher surface modulus than the second layer.

957) The method of any of embodiments 912-950 wherein the first layer has a greater toughness than the second layer.

958) The method of any of embodiments 912-950 wherein the first layer has a higher Tg than the second layer.

959) The method of any of embodiments 912-950 wherein the first layer has a thickness of 0.1 to 1.5 mm.

960) The method of any of embodiments 912-950 wherein the first layer has a thickness of 0.1 to 1.0 mm.

961) The method of any of embodiments 912-950 wherein the first layer has a thickness of 0.1 to 0.5 mm.

962) The method of any of embodiments 912-961 wherein the first layer contains a pharmaceutically active agent.

963) The method of any of embodiments 912-961 wherein the second layer contains a pharmaceutically active agent.

964) The method of any of embodiments 912-961 wherein the multilayer composition has a thickness of 1.0 to 3.0 mm.

965) The method of any of embodiments 912-961 wherein the multilayer composition has a thickness of 1.5 to 2.0 mm.

966) The method of any of embodiments 912-961 wherein the multilayer composition is marked.

967) The method of any of embodiments 912-961 wherein the multilayer composition is sterile.

968) The method of any of embodiments 912-961 wherein the multilayer composition has a longest straight line dimension of 1 to 5 cm or is in the form of a cap having a half-circumference of 10-200 mm.

969) The method of any of embodiments 912-968 wherein the first, the second and/or the third polymer is the reaction product of a pre-polymer and a diamine, where the pre-polymer is the reaction product of a diisocyanate and a polyetherdiol.

970) The method of embodiment 969 wherein the polyetherdiol comprises at least one type of oxyalkylene sequence selected from the group consisting of oxyethylene, oxypropylene, oxytrimethylene and oxytetramethylene sequences.

971) The method of any of embodiments 969 or 970 wherein the polyetherdiol is a blend of polyetherdiols.

972) The method of any of embodiments 969 or 970 wherein the polyether diol is not a blend of polyetherdiols.

973) The method of any of embodiments 969-972 wherein the polyetherdiol is a random copolymer of two or more oxyalkylene sequences.

974) The method of any of embodiments 969-972 wherein the polyetherdiol is a block copolymer of two or more oxyalkylene sequences.

975) The method of any of embodiments 969-972 wherein the polyetherdiol is an alternating copolymer of two or more oxyalkylene sequences.

976) The method of any of embodiments 969-972 wherein the polyetherdiol is an alternating copolymer of two different oxyalklylene repeat units, for example, oxyethylene and oxypropylene.

977) The method of any of embodiments 969-976 wherein the diamine is an aliphatic diamine.

978) The method of any of embodiments 969-976 wherein the diamine is a polyether diamine.

979) The method of any of embodiments 969-976 wherein the diamine is a blend of diamines.

980) The method of any of embodiments 969-976 wherein the diamine is a blend of aliphatic diamine and polyether diamine.

981) The method of any of embodiments 912-968 wherein the first, the second, and/or the third polymer is the reaction product of diisocyanate and a polyetherdiamine to form a pre-polymer, and the reaction product of the pre-polymer and a diol to form a polyether urea urethane.

982) The method of embodiment 981 wherein the polyetherdiamine comprises at least one type of oxyalkylene sequence selected from the group consisting of oxyethylene, oxypropylene, oxytrimethylene and oxytetramethylene sequences.

983) The method of any of embodiments 981 or 982 wherein the polyetherdiamine is a blend of polyetherdiamines.

984) The method of any of embodiments 981-983 wherein the polyetherdiamine is not a blend of polyetherdiamines.

985) The method of any of embodiments 981-983 wherein the polyetherdiamine is a random copolymer of two or more oxyalkylene sequences.

986) The method of any of embodiments 981-983 wherein the polyetherdiamine is a block copolymer of two or more oxyalkylene sequences.

987) The method of any of embodiments 981-983 wherein the polyetherdiamine is an alternating copolymer of two or more oxyalkylene sequences.

988) The method of any of embodiments 981-983 wherein the polyetherdiamine is an alternating copolymer of two different oxyalklylene repeat units, for example, oxyethylene and oxypropylene.

989) The method of any of embodiments 981-988 wherein the diol is an aliphatic diol.

990) The method of any of embodiments 981-988 wherein the diol is an aromatic diol.

991) The method of any of embodiments 981-988 wherein the diol is a polyether diol.

992) The method of any of embodiments 981-988 wherein the diol is a blend of diols.

993) The method of any of embodiments 981-988 wherein the diol is a blend of aliphatic diol and polyetherdiol.

994) The method of any of embodiments 981-993 wherein the diisocyanate is an aliphatic diisocyanate and the reactants do not include an aromatic diisocyanate to form a polyether urea urethane.

995) The method of any of embodiments 981-993 wherein the diisocyanate is a mixture of aliphatic diisocyanates and the reactants do not include an aromatic diisocyanate to form a polyether urea urethane.

996) The method of any of embodiments 981-993 wherein the diisocyanate is an aromatic diisocyanate and the reactants do not include an aliphatic diisocyanate to form a polyether urea urethane.

997) The method of any of embodiments 981-993 wherein the diisocyanate is a mixture of aromatic diisocyanates and the reactants do not include an aliphatic diisocyanate to form a polyether urea urethane.

998) The method of any of embodiments 981-993 wherein the diisocyanate is a mixture of aromatic diisocyanate and aliphatic diisocyanate to form a polyether urea urethane.

999) The method of any of embodiments 912-968 wherein the first and/or the third polymer is the reaction product of a diisocyanate and a diol.

1000) The method of embodiment 999 wherein the diol is a polyether diol.

1001) The method of embodiment 1000 wherein the polyether diol comprises at least one type of oxyalkylene sequence selected from the group consisting of oxyethylene, oxypropylene, oxytrimethylene and oxytetramethylene sequences.

1002) The method of any of embodiments 1000 or 1001 wherein the polyetherdiol is a blend of polyetherdiols.

1003) The method of any of embodiments 1000 or 1001 wherein the polyetherdiol is not a blend of polyetherdiols.

1004) The method of any of embodiments 1000-1003 wherein the polyetherdiol is a random copolymer of two or more oxyalkylene sequences.

1005) The method of any of embodiments 1000-1003 wherein the polyetherdiol is a block copolymer of two or more oxyalkylene sequences.

1006) The method of any of embodiments 1000-1003 wherein the polyetherdiol is an alternating copolymer of two or more oxyalkylene sequences.

1007) The method of any of embodiments 1000-1003 wherein the polyetherdiol is an alternating copolymer of two different oxyalklylene repeat units, for example, oxyethylene and oxypropylene.

1008) The method of any of embodiments 999-1007 wherein the diisocyanate is an aliphatic diisocyanate and the reactants do not include an aromatic diisocyanate.

1009) The method of any of embodiments 999-1007 wherein the diisocyanate is a mixture of aliphatic diisocyanates and the reactants do not include an aromatic diisocyanate.

1010) The method of any of embodiments 999-1007 wherein the diisocyanate is an aromatic diisocyanate and the reactants do not include an aliphatic diisocyanate.

1011) The method of any of embodiments 999-1007 wherein the diisocyanate is a mixture of aromatic diisocyanates and the reactants do not include an aliphatic diisocyanate.

1012) The method of any of embodiments 999-1007 wherein the diisocyanate is a mixture of aromatic diisocyanate and aliphatic diisocyanate.

1013) The method of any of embodiments 999-1012 wherein diisocyanate and diol are the only reactants.

1014) The method of any of embodiments 999-1013 wherein the molar ratio of diisocyanate to polyether diol is in the range of 0.95 to 1.05.

1015) The method of any of embodiments 912-968 wherein one or both of the first and third polymer is the reaction product of a diisocyanate and a diamine.

1016) The method of embodiment 1015 wherein the diamine is a polyether diamine.

1017) The method of embodiment 1016 wherein the polyether diamine comprises at least one type of oxyalkylene sequence selected from the group consisting of oxyethylene, oxypropylene, oxytrimethylene and oxytetramethylene sequences.

1018) The method of any of embodiments 1016 or 1017 wherein the polyetherdiamine is a blend of polyetherdiamines.

1019) The method of any of embodiments 1016 or 1017 wherein the polyether diamine is not a blend of polyetherdiamines.

1020) The method of any of embodiments 1016-1019 wherein the polyetherdiamine is a random copolymer of two or more oxyalkylene sequences.

1021) The method of any of embodiments 1016-1019 wherein the polyetherdiamine is a block copolymer of two or more oxyalkylene sequences.

1022) The method of any of embodiments 1016-1019 wherein the polyetherdiamine is an alternating copolymer of two or more oxyalkylene sequences.

1023) The method of any of embodiments 1016-1019 wherein the polyetherdiamine is an alternating copolymer of two different oxyalklylene repeat units, for example, oxyethylene and oxypropylene.

1024) The method of any of embodiments 1015-1023 wherein the diisocyanate is an aliphatic diisocyanate and the reactants do not include an aromatic diisocyanate.

1025) The method of any of embodiments 1015-1023 wherein the diisocyanate is a mixture of aliphatic diisocyanates and the reactants do not include an aromatic diisocyanate.

1026) The method of any of embodiments 1015-1023 wherein the diisocyanate is an aromatic diisocyanate and the reactants do not include an aliphatic diisocyanate.

1027) The method of any of embodiments 1015-1023 wherein the diisocyanate is a mixture of aromatic diisocyanates and the reactants do not include an aliphatic diisocyanate.

1028) The method of any of embodiments 1015-1023 wherein the diisocyanate is a mixture of aromatic diisocyanate and aliphatic diisocyanate.

1029) The method of any of embodiments 1015-1028 wherein diisocyanate and diamine are the only reactants.

1030) The method of any of embodiments 1016-1029 wherein the molar ratio of diisocyanate to polyether diamine is in the range of 0.95 to 1.05.

1031) The method of any of embodiments 912-968 wherein one or both of the first and third polymer is the reaction product of a diisocyanate and either (a) a mixture comprising polyether diol and polycarbonate diol or (b) a polyether polycarbonate diol.

1032) The method of embodiment 1031 wherein the polyetherdiol comprises at least one type of oxyalkylene sequence selected from the group consisting of oxyethylene, oxypropylene, oxytrimethylene and oxytetramethylene sequences.

1033) The method of any of embodiments 1031 or 1032 wherein the polyetherdiol is a blend of polyetherdiols.

1034) The method of any of embodiments 1031 or 1032 wherein the polyether diol is not a blend of polyetherdiols.

1035) The method of any one of embodiments 1031-1034 wherein the polyetherdiol is a random copolymer of two or more oxyalkylene sequences.

1036) The method of any one of embodiments 1031-1034 wherein the polyetherdiol is a block copolymer of two or more oxyalkylene sequences.

1037) The method of any of embodiments 1031-1034 wherein the polyetherdiol is an alternating copolymer of two or more oxyalkylene sequences.

1038) The method of any of embodiments 1031-1034 wherein the polyetherdiol is an alternating copolymer of two different oxyalklylene repeat units, for example, oxyethylene and oxypropylene.

1039) The method of any one of embodiments 1031-1038 wherein the polycarbonate diol is poly(hexamethylene carbonate)diol.

1040) The method of any one of embodiments 1031-1038 wherein the polycarbonate diol is poly(ethylene-carbonate)diol.

1041) The method of any one of embodiments 1031-1038 wherein the polycarbonate diol is the reaction product of trimethylene carbonate and a diol.

1042) The method of any one of embodiments 1031-1041 wherein the diisocyanate is an aliphatic diisocyanate and the reactants do not include an aromatic diisocyanate.

1043) The method of any one of embodiments 1031-1041 wherein the diisocyanate is a mixture of aliphatic diisocyanates and the reactants do not include an aromatic diisocyanate.

1044) The method of any one of embodiments 1031-1041 wherein the diisocyanate is an aromatic diisocyanate and the reactants do not include an aliphatic diisocyanate.

1045) The method of any one of embodiments 1031-1041 wherein the diisocyanate is a mixture of aromatic diisocyanates and the reactants do not include an aliphatic diisocyanate.

1046) The method of any one of embodiments 1031-1041 wherein the diisocyanate is a mixture of aromatic diisocyanate and aliphatic diisocyanate.

1047) The method of any one of embodiments 1031-1046 further comprising chain extending by reaction with a diol.

1048) The method of any of embodiments 912-968 wherein one or both of the first and second polymer is the reaction product of a diamine and a pre-polymer, where the pre-polymer is the reaction product of a diisocyanate and either (a) a mixture comprising polyether diol and polycarbonate diol or (b) a polyether polycarbonate diol.

1049) The method of embodiment 1048 wherein the polyetherdiol comprises at least one type of oxyalkylene sequence selected from the group consisting of oxyethylene, oxypropylene, oxytrimethylene and oxytetramethylene sequences.

1050) The method of any of embodiments 1048 or 1049 wherein the polyetherdiol is a blend of polyetherdiols.

1051) The method of any of embodiments 1048 or 1049 wherein the polyether diol is not a blend of polyetherdiols.

1052) The method of any one of embodiments 1048-1051 wherein the polyetherdiol is a random copolymer of two or more oxyalkylene sequences.

1053) The method of any one of embodiments 1048-1051 wherein the polyetherdiol is a block copolymer of two or more oxyalkylene sequences.

1054) The method of any of embodiments 1048-1051 wherein the polyetherdiol is an alternating copolymer of two or more oxyalkylene sequences.

1055) The method of any of embodiments 1048-1051 wherein the polyetherdiol is an alternating copolymer of two different oxyalklylene repeat units, for example, oxyethylene and oxypropylene.

1056) The method of any one of embodiments 1048-1055 wherein the polycarbonate diol is poly(hexamethylene carbonate)diol.

1057) The method of any one of embodiments 1048-1055 wherein the polycarbonate diol is poly(ethylene-carbonate)diol.

1058) The method of any one of embodiments 1048-1055 wherein the polycarbonate diol is the reaction product of trimethylene carbonate and a diol.

1059) The method of any one of embodiments 1048-1058 wherein the diisocyanate is an aliphatic diisocyanate and the reactants do not include an aromatic diisocyanate.

1060) The method of any one of embodiments 1048-1058 wherein the diisocyanate is a mixture of aliphatic diisocyanates and the reactants do not include an aromatic diisocyanate.

1061) The method of any one of embodiments 1048-1058 wherein the diisocyanate is an aromatic diisocyanate and the reactants do not include an aliphatic diisocyanate.

1062) The method of any one of embodiments 1048-1058 wherein the diisocyanate is a mixture of aromatic diisocyanates and the reactants do not include an aliphatic diisocyanate.

1063) The method of any one of embodiments 1048-1058 wherein the diisocyanate is a mixture of aromatic diisocyanate and aliphatic diisocyanate.

1064) The method of any one of embodiments 1048-1063 wherein the diamine is an aliphatic diamine.

1065) The method of any one of embodiments 1048-1063 wherein the diamine is a polyether diamine.

1066) The method of any one of embodiments 1048-1063 wherein the diamine is a blend of diamines.

1067) The method of any one of embodiments 1048-1063 wherein the diamine is a blend of aliphatic diamine and polyether diamine.

1068) The method of any of embodiments 912-968 wherein one or both of the first and third polymer is the reaction product of a diisocyanate and either (a) a mixture comprising polyether diol and polyester diol or (b) a polyether polyester diol.

1069) The method of embodiment 1068 wherein the polyether diol comprises at least one type of oxyalkylene sequence selected from the group consisting of oxyethylene, oxypropylene, oxytrimethylene and oxytetramethylene sequences.

1070) The method of any of embodiments 1068 or 1069 wherein the polyether diol is a blend of polyetherdiols.

1071) The method of any of embodiments 1068 or 1069 wherein the polyether diol is not a blend of polyetherdiols.

1072) The method of any one of embodiments 1068-1071 wherein the polyether diol is a random copolymer of two or more oxyalkylene sequences.

1073) The method of any one of embodiments 1068-1071 wherein the polyether diol is a block copolymer of two or more oxyalkylene sequences.

1074) The method of any of embodiments 1068-1071 wherein the polyetherdiol is an alternating copolymer of two or more oxyalkylene sequences.

1075) The method of any of embodiments 1068-1071 wherein the polyetherdiol is an alternating copolymer of two different oxyalklylene repeat units, for example, oxyethylene and oxypropylene.

1076) The method of any one of embodiments 1068-1075 wherein the diisocyanate is an aliphatic diisocyanate and the reactants do not include an aromatic diisocyanate.

1077) The method of any one of embodiments 1068-1075 wherein the diisocyanate is a mixture of aliphatic diisocyanates and the reactants do not include an aromatic diisocyanate.

1078) The method of any one of embodiments 1068-1075 wherein the diisocyanate is an aromatic diisocyanate and the reactants do not include an aliphatic diisocyanate.

1079) The method of any one of embodiments 1068-1075 wherein the diisocyanate is a mixture of aromatic diisocyanates and the reactants do not include an aliphatic diisocyanate.

1080) The method of any one of embodiments 1068-1075 wherein the diisocyanate is a mixture of aromatic diisocyanate and aliphatic diisocyanate.

1081) The method of any of embodiments 912-968 wherein one or both of the first and third polymer is the reaction product of a diamine and a pre-polymer, where the pre-polymer is the reaction product of a diisocyanate and either (a) a mixture comprising polyether diol and polyester diol or (b) a polyether polyester diol.

1082) The method of embodiment 1081 wherein the polyetherdiol comprises at least one type of oxyalkylene sequence selected from the group consisting of oxyethylene, oxypropylene, oxytrimethylene and oxytetramethylene sequences.

1083) The method of one of embodiments 1081 or 1082 wherein the polyetherdiol is a blend of polyetherdiols.

1084) The method of any one of embodiments 1081 or 1082 wherein the polyether diol is not a blend of polyetherdiols.

1085) The method of any one of embodiments 1081-1084 wherein the polyetherdiol is a random copolymer of two or more oxyalkylene sequences.

1086) The method of any one of embodiments 1081-1084 wherein the polyetherdiol is a block copolymer of two or more oxyalkylene sequences.

1087) The method of any of embodiments 1081-1084 wherein the polyetherdiol is an alternating copolymer of two or more oxyalkylene sequences.

1088) The method of any of embodiments 1081-1084 wherein the polyetherdiol is an alternating copolymer of two different oxyalklylene repeat units, for example, oxyethylene and oxypropylene.

1089) The method of any one of embodiments 1081-1088 wherein the diisocyanate is an aliphatic diisocyanate and the reactants do not include an aromatic diisocyanate.

1090) The method of any one of embodiments 1081-1088 wherein the diisocyanate is a mixture of aliphatic diisocyanates and the reactants do not include an aromatic diisocyanate.

1091) The method of any one of embodiments 1081-1088 wherein the diisocyanate is an aromatic diisocyanate and the reactants do not include an aliphatic diisocyanate.

1092) The method of any one of embodiments 1081-1088 wherein the diisocyanate is a mixture of aromatic diisocyanates and the reactants do not include an aliphatic diisocyanate.

1093) The method of any one of embodiments 1081-1088 wherein the diisocyanate is a mixture of aromatic diisocyanate and aliphatic diisocyanate.

1094) The method of any one of embodiments 1081-1093 wherein the diamine is an aliphatic diamine.

1095) The method of any one of embodiments 1081-1093 wherein the diamine is a polyether diamine.

1096) The method of any one of embodiments 1081-1093 wherein the diamine is a blend of diamines.

1097) The method of any one of embodiments 1081-1093 wherein the diamine is a blend of aliphatic diamine and polyether diamine.

1098) The method any of one of embodiments 912-1097 wherein at least one of the first and third polymer is bio-stable.

1099) The method any of one of embodiments 912-1097 wherein at least one of the first and third polymers absorbs at least 50% of its weight in water when immersed in 1% aqueous methyl cellulose at 37° C. for 16 hours.

1100) The method any of one of embodiments 912-1097 wherein at least one of the first and third polymers has a COF of 0.001 to 0.15.

1101) The method any of one of embodiments 912-1097 wherein at least one of the first and third polymers has an intrinsic viscosity of 3-8 dl/g.

1102) A method comprising placing a multilayer composition according to any of embodiments 1-518 into a pre-selected location in a subject in need thereof.

1103) A method comprising placing a multilayer composition prepared according to the methods of any of embodiments 519-1101 into a pre-selected location in a subject in need thereof.

1104) A method comprising

(a) receiving the dimensions of a bone in a subject; and

(b) preparing a multilayer composition according to any of embodiments 1-518 that fits snugly on the bone.

1105) A method comprising

(a) receiving the dimensions of a bone in a subject; and

(b) preparing a multilayer composition according to the method of any of embodiments 519-1101 that fits snugly on the bone.

1106) A kit comprising

(a) a multilayer composition according to any of embodiments 1-518; and

(b) a solution that comprises an anesthetic.

1107) A multilayer composition comprising

(a) a first layer, a second layer and a third layer, the second layer being located intermediate between the first and second layers, each layer having a compressive modulus and a hydrophilicity;

(b) the compressive modulus of the first layer being greater than the compressive modulus of the second layer, and the compressive modulus of the second layer being greater than the compressive modulus of the third layer; and

(c) the hydrophilicity of the first layer being less than the hydrophilicity of the second layer, and the hydrophilicity of the second layer being less than the hydrophilicity of the third layer.

1108) The composition of embodiment 1107 having a thickness in the range of 0.5 to 4.0 mm.

1109) The composition of embodiment 1107, where each of the first, second and third layers comprises a polymer that comprises urethane or urea groups.

1. A multilayer composition in the form of a cap, the cap comprising a center and a rim around the center, the cap comprising an interior surface and an exterior surface, at least one of and preferably both of the center and the rim comprising a first layer in contact with a second layer, the interior surface comprising the first layer, the first layer comprising a first polymer, the second layer comprising a second polymer, the first polymer comprising a plurality of urethane or urea groups, the second polymer comprising a plurality of urethane or urea groups, the first and second polymers being non-identical.

2. A multilayer composition in the form of a cap, the cap comprising a center and a rim around the center, the cap comprising an interior surface and an exterior surface, at least one of, and preferably both of the center and the rim comprising a first layer in contact with a second layer, the interior surface comprising the first layer, the first layer comprising a blend of polymers, the second layer comprising a second different blend of polymers, the polymers of the blend forming the first layer comprising a plurality of urethane or urea groups, the polymers of the blend forming the second layer comprising a plurality of urethane or urea groups, the first and second polymer blends being non-identical.

3. The composition of claim 1 wherein the cap has a longest exterior dimension of 10 to 200 mm, the center has a thickness of 1.5 to 2.0 mm, and the rim has a thickness of 0.5 to 1.5 mm.

4. The composition of claim 1 wherein the first layer has a compressive modulus ranging from 0.1-1.0 MPa, and the second layer has a compressive modulus ranging from 1.0-10 MPa.

5. The composition of claim 1 wherein the first layer makes up 10-20% of the thickness of the construct, and the second layer makes up 80-90% of the thickness of the construct.

6. The composition of claim 1 wherein the first layer relative to the second layer is at least one of:
(a) is less hydrophilic;
(b) has a greater hardness;
(c) has a higher compressive modulus; and/or
(d) has a higher Tg.

7. The composition of claim 1 having exactly two layers.

8. The composition of claim 1 wherein the second polymer contains a greater weight percent of ether linkages compared to the first polymer, and wherein the second polymer is more hydrophilic than the first polymer.

9. The composition of claim 1 wherein the second layer comprises a blend of polymers, the combination of which has a greater weight percent of ether linkages compared to the blend of polymers in the first layer, and wherein the second layer is more hydrophilic than the first layer.

10. The composition of claim 1 wherein the inner surface contains a pharmaceutically active agent.

11. The composition of claim 1 further comprising a third layer in contact with the second layer, the third layer comprising a third polymer, the third polymer comprising a plurality of urethane groups, the second and third polymers being non-identical.

12. (canceled)

13. The composition of claim 1 wherein both of the cap and the rim has exactly three layers.

14. (canceled)

15. An assembly comprising a multilayer sheet-like composition in the form of a cap in combination with a support, the support selected from the group consisting of a bone and a material in the shape of a bone, wherein the cap fits snugly around a portion of the support, the cap comprising a center and a rim around the center, the cap comprising an interior surface and an exterior surface, at least one of the center and the rim comprising a first layer in contact with a second layer, the interior surface comprising the first layer, the first layer comprising a first polymer or blend of polymers, the second layer comprising a second polymer or a second blend of polymers, the first polymer or first blend of polymers comprising a plurality of urethane or urea groups, the second polymer or second blend of polymers comprising a plurality of urethane or urea groups, the first and second polymers or blends of polymers being non-identical.

16. The assembly of claim 15 wherein the support is in the shape of a head of a femur bone.

17-26. (canceled)
